(19) United States
(12) Patent Application Publication
(10) Pub. No.: US 2010/0240642 A1
(43) Pub. Date: Sep. 23, 2010

(54) 1H-Indole-2-Carboxylic Acid Derivatives Useful As PPAR Modulators

(75) Inventors: Jeffrey Alan Oplinger, Durham, NC (US); Paul Kenneth Spearing, Durham, NC (US); Millard Hurst Lambert, Durham, NC (US); John A. Ray, Durham, NC (US); Christopher P. Laudeman, Durham, NC (US); Jerzy R. Szewczyk, Durham, NC (US); Pierette Banker, Durham, NC (US)

Correspondence Address: GLAXOSMITHKLINE;GLOBAL PATENTS, C.2111F, FIVE MOORE DR., PO BOX 13398, RESEARCH TRIANGLE PARK, NC, 27709-3398, US

(73) Assignee: SMITHKLINE BEECHAM CORPORATION, PHILADELPHIA, PA (US)

(21) Appl. No.: 12/438,024

(22) PCT Filed: Aug. 31, 2007
(86) PCT No.: PCT/US07/77365

§ 371 Date: Feb. 19, 2009

Related U.S. Application Data
(60) Provisional application No. 60/824,120, filed on Aug. 31, 2006.

(51) Int. Cl. A61K 31/404; C07D 209/42; C07D 401/04; C07D 413/10; C07D 409/12; C07D 403/10; C07D 417/10; A61K 31/4439; A61K 31/5377; A61K 31/496; A61K 31/541; A61P 3/10

(52) U.S. Cl. 514/228.2; 548/492; 546/278.1; 544/143; 548/465; 544/373; 544/58.5; 544/58.2; 514/419; 514/339; 514/235.2; 514/414; 514/254.09

(57) ABSTRACT

The present invention relates to certain indole derivatives that are modulators of PPARγ, to processes for their preparation, to pharmaceutical compositions containing them and to their use in medicine.

### FIELD OF THE INVENTION

The present invention relates to certain novel compounds, to processes for their preparation, to pharmaceutical compositions containing them and to their use in medicine. More specifically, this invention relates to indole derivatives that are modulators of PPARγ, and also to the methods for the making and use of such compounds.

### BACKGROUND OF THE INVENTION

Treatment of type 2 diabetes mellitus (T2DM) usually begins with a combination of diet and exercise, with progression to oral hypoglycemic (e.g. sulfonylureas) and in more severe cases, insulin. More recently, a class of compounds known as thiazolidinediones (e.g. U.S. Pat. Nos. 5,089,514, 4,342,771, 4,367,234, 4,340,605, 5,306,726) have emerged as effective antidiabetic agents that enhance the insulin sensitivity of target tissues (skeletal muscle, liver, adipose) in animal models of type 2 diabetes mellitus and also reduce lipid and insulin levels in these animal models.

It has been reported that thiazolidinediones are potent and selective activators of PPARγ and bind directly to the PPARγ receptor (J. M. Lehmann et. al., J. Biol. Chem. 12953-12956, 270 (1995)), providing evidence that PPARγ is a possible target for the therapeutic actions of the thiazolidinediones.

Activators of the nuclear receptor PPARγ, for example troglitazone, have been shown in the clinic to enhance insulin-action, reduce serum glucose and have small but significant effects on reducing serum triglyceride levels in patients with type 2 diabetes. See, for example, D. E. Kelly et al., Curr. Opin. Endocrinol. Diabetes, 90-96, 5 (2), (1998); M. D. Johnson et al., Ann. Pharmacother., 337-348, 32 (3), (1997); and M. Leutenegger et al., Curr. Ther. Res., 403-416, 58 (7), (1997). More recently rosiglitazone and pioglitazone have entered widespread clinical use and have been shown to be effective agents to treat type 2 diabetes. These ligands are considered full agonists of the PPARγ nuclear receptor that regulate many genes thought to be involved in glucose and lipid homeostasis. Unfortunately, their efficacy is limited in many patients due to adverse events (AE's), principally fluid retention and weight gain. While the exact cause of the AE's produced by PPARγ full agonist compounds is not completely understood, evidence is emerging that suggests partial activation of the PPARγ receptor may provide the desired effects on glucose homeostasis and avoid or diminish the AE's associated with full agonist therapy. Human clinical experience with a putative PPARγ partial agonist (MBX-102 from Metabolex) has revealed that short term therapy in type 2 diabetic patients with this agent was effective in reducing plasma glucose levels without weight gain or increased fluid retention (Rosenstock, J. et. al. American Diabetes Association Annual Meeting, June 2005, San Diego, Calif., Abstract No. 44-OR.). Also, a putative PPARγ partial agonist, T131, increased levels of adiponectin, a marker of PPARγ activation, in healthy human volunteers, without weight gain or increases in markers of fluid retention (Motanao, N. et. al. Abstracts of Papers, 231st ACS National Meeting, Atlanta, Ga., United States, Mar. 26-30, 2006, MEDI-020).

In addition to their strong insulin sensitizing effects, PPARγ ligands have demonstrated the potential to have a positive effect in a number of chronic inflammation related disorders. Recent findings have linked PPARγ activation to a favorable modulation of Alzhiemers disease pathophysiology in a process potentially mediated via PPARγ-evoked repression of the beta-site amyloid precursor protein-cleaving enzyme (BACE1). (see for example Combs, C. K. et. al. J. Neurosci 2000, 20, 558-67; Sastre, M. et al. Proc Natl Acad Sci USA 2006, 103(2): 443). Rheumatoid arthritis is a chronic inflammatory disease of the joint with massive synovial proliferation and angiogenesis. Thus, the ability of PPARγ agonists to suppress macrophage activation and expression of pro-inflammatory genes suggests utility for such agonist compounds in the treatment of rheumatoid arthritis (see Cheon, J. D. et. al. J. Autoimmun 2001, 17, 215-21).

PPARγ is expressed in many cell types throughout the vasculature including smooth muscle cells, endothelial cells and macrophages. Activation of PPARγ has resulted in reduced smooth muscle cell migration and proliferation, a reduction in pro-inflammatory cytokines, and improvements in endothelial function (via increased NO release) that may contribute to improvements in conditions of the atherosclerosis disease state (see for example Palinski, W and Li, A. C. in Annu. Rev. Pharmacol. Toxicol 2006, 46(1), 1-39; Staels, B. Current Medical Research and Opinion 2005, 21(Suppl 1), S13-S20; Simonson, G. D. and Kendall, D. M. Curr. Opin Endocrinol Diabetes 2006, 13, 162-170; Babaev, Vladimir R.; Yancey, Patricia G.; Ryzhov, Sergey V.; Kon, Valentina; Breyer, Matthew D.; Magnuson, Mark A.; Fazio, Sergio; Linton, MacRae F. Arteriosclerosis, Thrombosis, and Vascular Biology 2005, 25(8), 1647-1653). Fatty liver disease and inflammatory digestive diseases such as ulcerative colitis and Crohn's disease may also be positively impacted with administration of PPARγ activators (therapeutic potential of PPARγ agonists review: Motilva, V. et. al. Current Pharmaceutical Design 2004, 10, 3505-3524).

PPARs and PPARγ ligands in particular have been implicated as important regulators in cell differentiation and as such may offer potential as effective anticancer agents (see for example Koichi, M. et al International Journal of Oncology 2004, 25(3), 631-639; Charles, C. Anticancer Research 2004, 24(5A), 2765-2771; Kinoshita, Y. Current Medicinal Chemistry: Anti-Cancer Agents 2004, 4(6), 465-477).

Recent reports have disclosed PPARγ partial agonist compounds. For example, an indole compound is disclosed in WO 2001/30343. A series of indole compounds are disclosed in WO 2002/08188, WO 2004/020408, WO 2004/020409, and WO 2004/019869. WO 2004/066963 describes a series of N-cyclohexylaminocarbonyl benzenesulfonamide derivatives. Pyrazole derivatives are disclosed in WO 2004/043951. Benzimidazole compounds are disclosed in WO 1997/24334, WO 1999/00373, and WO 2000/39099. PPARγ partial agonist FK614 has been reported (European J. of Pharm. 494 (2004) 273-281; WO2004005550). A series of 4H-benzo(1,4)oxazin-3-ones are disclosed in WO 2001/87862. A series of vinyl N-(2-benzoylphenyl)-L-tyrosine derivatives are disclosed in US 2003/109560. A series of acylsulfamides are disclosed in WO 2002/060388. Pyrazolo[1,5-a]pyrimidines are disclosed in WO 2003/053976. TZD analogue PAT5A has recently been disclosed as a partial agonist of PPARγ (Misra, P. et. al. The Journal of Pharmacology and Experimental Therapeutics 2004, 306(2), 763-771).

### SUMMARY OF INVENTION

Briefly, in one aspect, the present invention provides compounds of formula (I)

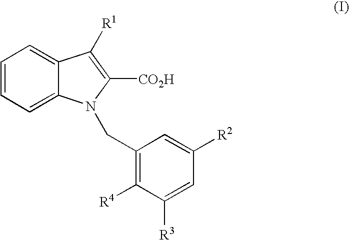

or salt or solvate thereof, wherein;
R¹ is —O-Ph-C₁-₆alkyl, —NH-Ph-C₁-₆alkyl, —CH₂-Ph-haloC₁-₆alkyl, aryl or heterocyclyl, wherein said aryl or heterocyclyl is optionally mono-substituted with R⁷;
R² is C₁-₆haloalkyl, R^{a}—R^{b}—R^{c}, heterocyclyl or aryl, wherein said aryl is optionally substituted with R⁸ and said heterocyclyl is optionally substituted with R⁹;
R³ is H, C₁-₆haloalkyl, or R^{a}—R^{b}—R^{c};
R^{a} is —O—;
R^{b} is a bond, C₁-₆alkylene or —C(O)—;
R^{c} is H, C₁-₆alkyl, aryl, C₃₋₆cycloalkyl, C₁-₆alkoxy, —NR⁵R⁶, —O(CH₂)₂OCH₃, or heterocyclyl optionally substituted with ═O or C₁₋₆alkyl; wherein
when R^{b} is a bond, R^{c} is H or C₁-₆alkyl;
R⁴ and R⁵ are each independently H or C₁-₆alkyl; wherein
when R³ and R⁴ are both H, R² is optionally substituted aryl or optionally substituted heterocyclyl;
R⁶ is C₁-₆alkyl, or thienylC₁-₆alkylene;
R⁷ is C₁-₆alkyl, —C(O)CH₃, C₁-₆alkoxy, or haloC₁-₆alkyl;
R⁸ is —OH, —CO₂H, —OC₁-₆alkylenephenyl, C₁-₆alkoxy, —SC₁-₆alkyl, —S(O)₂C₁-₆alkyl, —C(O)NR⁵R⁶, or —OC(CH₃)₂CO₂H; and
R⁹ is —C(O)CH₃, —C(O)OC₁-₆alkyl, —C(O)O(CH₂)₂OCH₃, —C(O)NH₂, —S(O)₂C₁-₆alkyl, —S(O)₂NH₂, or —S(O)₂NC(O)OC₁-₆alkyl.

Another aspect of the present invention provides a compound substantially as hereinbefore defined with reference to any one of the Examples.

Another aspect of the present invention provides a compound of the present invention that is a PPARγ modulator.

Another aspect of the present invention provides a pharmaceutical composition comprising a compound of the present invention and a pharmaceutically acceptable carrier.

Another aspect of the present invention provides a compound of the present invention for use as an active therapeutic substance.

Another aspect of the present invention provides a compound of the present invention for use in the treatment of hyperglycemia, type 2 diabetes, impaired glucose tolerance, insulin resistance, syndrome X, and dyslipidemia.

Another aspect of the present invention provides the use of a compound of the present invention in the manufacture of a medicament for use in the treatment of hyperglycemia, type 2 diabetes, impaired glucose tolerance, insulin resistance, syndrome X, and dyslipidemia.

Another aspect of the present invention provides a method for the treatment of hyperglycemia, type 2 diabetes, impaired glucose tolerance, insulin resistance, syndrome X, and dyslipidemia comprising the administration of a compound of the present invention.

### DETAILED DESCRIPTION OF THE PREFERRED EMBODIMENT

Terms are used within their accepted meanings. The following definitions are meant to clarify, but not limit, the terms defined.

As used herein the term “alkyl” refers to a straight or branched chain hydrocarbon, preferably having from one to six carbon atoms. Examples of “alkyl” as used herein include, but are not limited to, methyl, ethyl, propyl, isopropyl, isobutyl, n-butyl, t-butyl, isopentyl, and n-pentyl.

As used throughout this specification, the preferred number of atoms, such as carbon atoms, will be represented by, for example, the phrase “Cₓ₋C_{y} alkyl,” which refers to an alkyl group, as herein defined, containing the specified number of carbon atoms. Similar terminology will apply for other preferred terms and ranges as well.

As used herein, the term “alkylene” refers to a straight or branched chain divalent hydrocarbon radical, preferably having from one to six carbon atoms. Examples of “alkylene” as used herein include, but are not limited to, methylene (—CH₂—), ethylene (—CH₂—CH₂—), and branched versions thereof such as (—CH(CH₃)—) and the like.

As used herein, the term “cycloalkyl” refers to a non-aromatic cyclic hydrocarbon ring. Exemplary “cycloalkyl” groups include, but are not limited to, cyclopropyl, cyclobutyl, cyclopentyl, cyclohexyl, cycloheptyl, and the like.

As used herein, the term “heterocycle” or “heterocyclyl” refers to a mono- or poly-cyclic ring system containing one or more heteroatoms and optionally containing one or more degrees of unsaturation, including monocyclic five to seven membered aromatic or non-aromatic rings, or a fused bicyclic aromatic or non-aromatic ring system comprising two of such rings. Preferred heteroatoms include N, O, and S, where N-oxides, sulfur oxides, and sulfur dioxides are permissible heteroatom substitutions. Preferably the ring is three to ten-membered. Such rings may be optionally fused to one or more of another “heterocycle” ring(s), “aryl” ring(s), or “cycloalkyl” ring(s). Examples of “heterocycle” groups include, but are not limited to, benzofurane, thiophene, pyridine, morpholine, thiomorpholine, dioxidothiomorpholine, piperazine, imidazolidine, piperidine, pyrrolidine, and pyrrole, and the like. Preferred heterocyclyl groups include benzofuranyl, thiophenyl, pyridinyl, morpholinyl, thiomorpholinyl, dioxidothiomorpholinyl, piperazinyl, imidazolidinyl, piperidinyl, pyrrolidinyl, and pyrrolyl.

As used herein, the term “aryl” refers to a benzene ring or to a fused benzene ring system, for example anthracene, phenanthrene, or naphthalene ring systems. Examples of “aryl” groups include, but are not limited to, phenyl, 2-naphthyl, 1-naphthyl, biphenyl, and the like. One preferred aryl group is phenyl.

As used herein the term “halogen” refers to fluorine, chlorine, bromine, or iodine.

As used herein the term “haloalkyl” refers to an alkyl group, as defined herein that is substituted with at least one halogen. Examples of branched or straight chained “haloalkyl” groups useful in the present invention include, but are not limited to, methyl, ethyl, propyl, isopropyl, n-butyl, and t-butyl substituted independently with one or more halogens, e.g., fluoro, chloro, bromo, and iodo. The term “haloalkyl” should be interpreted to include such substituents such as —CF₃, —CH₂—CH₂—F, —CH₂—CF₃, and the like.

As used herein the term “hydroxy” or “hydroxyl” refers to a group —OH.

As used herein, the term “oxo” refers to a group ═O

As used herein the term “alkoxy” refers to a group —ORₐ, where Rₐ is alkyl as herein defined.

As used herein the term “thienylalkylene” refers to a group —Rₐ—R_{b} wherein Rₐ is an alkylene group as herein defined, and R_{b} is a thienyl group.

As used herein throughout the present specification, the phrase “optionally substituted” or variations thereof denote an optional substitution, including multiple degrees of substitution, with one or more substituent group, preferably one or two. The phrase should not be interpreted so as to be imprecise or duplicative of substitution patterns herein described or depicted specifically. Rather, those of ordinary skill in the art will appreciate that the phrase is included to provide for obvious modifications, which are encompassed within the scope of the appended claims.

In one embodiment of the present invention is a compound of formula (II)

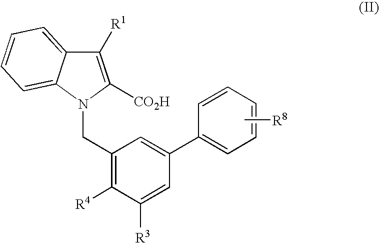

or a salt or solvate thereof, wherein
R¹ is —O-Ph-C₁-₆alkyl, —NH-Ph-C₁-₆alkyl, —CH₂-Ph-haloC₁-₆alkyl, aryl or heterocyclyl, wherein said aryl or heterocyclyl is optionally mono-substituted with R⁷;
R³ is H, C₁-₆haloalkyl, or R^{a}—R^{b}—R^{c};
R^{a} is —O—;
R^{b} is a bond, C₁-₆alkylene or —C(O)—;
R^{c} is H, C₁-₆alkyl, aryl, C₃₋₆cycloalkyl, C₁-₆alkoxy, —NR⁵R⁶, —O(CH₂)₂OCH₃, or heterocyclyl optionally substituted with ═O or C₁₋₆alkyl; wherein
when R^{b} is a bond, R^{c} is H or C₁-₆alkyl;
R⁴ and R⁵ are each independently H or C₁-₆alkyl;
R⁶ is C₁-₆alkyl, or thienylC₁-₆alkylene;
R⁷ is C₁-₆alkyl, —C(O)CH₃, C₁-₆alkoxy, or haloC₁-₆alkyl; and
R⁸ is —OH, —CO₂H, —OC₁-₆alkylenephenyl, C₁-₆alkoxy, —SC₁-₆alkyl, —S(O)₂C₁-₆alkyl, —C(O)NR⁵R⁶, or —OC(CH₃)₂CO₂H.

In another embodiment of the present invention is a compound of formula (III)

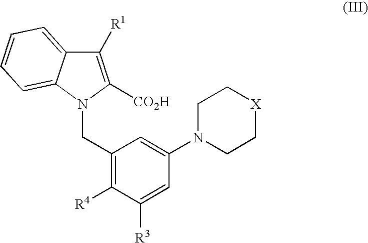

or a salt or solvate thereof, wherein
X is O, S, S(O)₂, or N—R⁹;
R¹ is —O-Ph-C₁-₆alkyl, —NH-Ph-C₁-₆alkyl, —CH₂-Ph-haloC₁-₆alkyl, aryl or heterocyclyl, wherein said aryl or heterocyclyl is optionally mono-substituted with R⁷;
R³ is H, OH, C₁-₆haloalkyl, C₁-₆alkoxy, or R^{a}—R^{b}—R^{c};
R^{a} is —O—;
R^{b} is a bond, C₁-₆alkylene or —C(O)—;
R^{c} is H, C₁-₆alkyl, aryl, C₃₋₆cycloalkyl, C₁-₆alkoxy, —NR⁵R⁶, —O(CH₂)₂OCH₃, or heterocyclyl optionally substituted with ═O or C₁₋₆alkyl; wherein
when R^{b} is a bond, R^{c} is H or C₁-₆alkyl;
R⁴ and R⁵ are each independently H or C₁-₆alkyl;
R⁶ is C₁-₆alkyl, or thienylC₁-₆alkylene;
R⁷ is C₁-₆alkyl, —C(O)CH₃, C₁-₆alkoxy, or haloC₁-₆alkyl; and
R⁹ is —C(O)CH₃, —C(O)OC₁-₆alkyl, —C(O)O(CH₂)₂OCH₃, —C(O)NH₂, —S(O)₂C₁-₆alkyl, —S(O)₂NH₂, or —S(O)₂NC(O)OC₁-₆alkyl.

In another embodiment of the present invention is a compound of formula (IV)

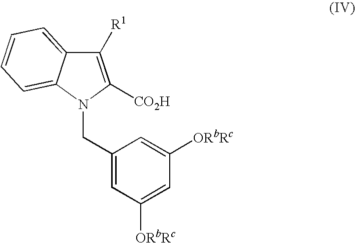

or a salt or solvate thereof, wherein
R¹ is —O-Ph-C₁-₆alkyl, —NH-Ph-C₁-₆alkyl, —CH₂-Ph-haloC₁-₆alkyl, aryl or heterocyclyl, wherein said aryl or heterocyclyl is optionally mono-substituted with R⁷;
R^{b} is a bond, C₁-₆alkylene or —C(O)—;
R^{c} is H, C₁-₆alkyl, aryl, C₃₋₆cycloalkyl, C₁-₆alkoxy, —NR⁵R⁶, —O(CH₂)₂OCH₃, or heterocyclyl optionally substituted with ═O or C₁₋₆alkyl; wherein
when R^{b} is a bond, R^{c} is H or C₁-₆alkyl;
R⁵ is H or C₁-₆alkyl;
R⁶ is C₁-₆alkyl, or thienylC₁-₆alkylene; and
R⁷ is C₁-₆alkyl, —C(O)CH₃, C₁-₆alkoxy, or haloC₁-₆alkyl.

In another embodiment of the present invention is a compound of formula (V)

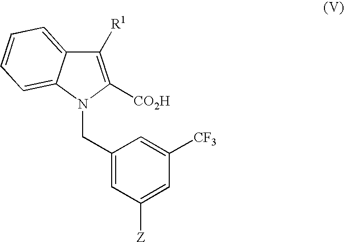

or a salt or solvate thereof, wherein
Z is CF₃ or OR^{b}R^{c};
R¹ is —O-Ph-C₁-₆alkyl, —NH-Ph-C₁-₆alkyl, —CH₂-Ph-haloC₁-₆alkyl, aryl or heterocyclyl, wherein said aryl or heterocyclyl is optionally mono-substituted with R⁷;
R^{b} is C₁-₆alkylene or —C(O)—;
R^{c} is H, C₁-₆alkyl, aryl, C₃₋₆cycloalkyl, C₁-₆alkoxy, —NR⁵R⁶, —O(CH₂)₂OCH₃, or heterocyclyl optionally substituted with ═O or C₁₋₆alkyl;
R⁵ is H or C₁-₆alkyl;
R⁶ is C₁-₆alkyl, or thienylC₁-₆alkylene; and
R⁷ is C₁-₆alkyl, —C(O)CH₃, C₁-₆alkoxy, or haloC₁-₆alkyl.

In another embodiment of the present invention is a compound of formulae I, II, III, IV or V, wherein R¹ is —O-Ph-t-butyl, —NH-Ph-t-butyl, —CH₂-Ph-CF₃, phenyl, benzofuranyl, thiophenyl, or pyridinyl, wherein said phenyl, benzofuranyl, thiophenyl, or pyridinyl, is optionally mono-substituted with R⁷.

In another embodiment of the present invention R^{c} is C₁-₆alkyl, phenyl, cyclopropyl, CF₃, —NR⁵R⁶, —O(CH₂)₂OCH₃, oxoimidazolidinyl, piperazinyl, piperidinyl, morpholinyl, pyrrolyl, or pyrrolidinyl, wherein said piperazinyl, piperidinyl, morpholinyl, pyrrolyl, or pyrrolidinyl is optionally substituted with C₁₋₆alkyl.

In another embodiment of the present invention R² is OH, C₁-₆alkoxy, CF₃, R^{a}—R^{b}—R^{c}, phenyl, morpholinyl, piperazinyl, thiomorpholinyl, or dioxidothiomorpholinyl, wherein said phenyl is optionally substituted with R⁸ and said morpholinyl, piperazinyl, thiomorpholinyl, or dioxidothiomorpholinyl is optionally substituted with R⁹.

In another embodiment of the present invention R¹ is optionally substituted phenyl. In another embodiment, R¹ is phenyl optionally substituted with C₁-₆alkyl. In a further embodiment, R¹ is phenyl optionally substituted with t-butyl.

In another embodiment of the present invention at least one of R² and R³ is R^{a}—R^{b}—R^{c}.

In another embodiment of the present invention R^{a} is —O—, R^{b} is C₁₋₃alkylene, and R^{c} is C₁₋₃alkoxy. In a further embodiment, R^{b} is ethylene and R^{c} is methoxy.

Suitable compounds of the present invention include:
1-({3-[(cyclopropylmethyl)oxy]-5-[(phenylmethyl)oxy]phenyl}methyl)-3-[4-(1,1-dimethylethyl)phenyl]-1H-indole-2-carboxylic acid;
1-[(3-[(cyclopropylmethyl)oxy]-5-{[2-(methyloxy)ethyl]oxy}phenyl)methyl]-3-[4-(1,1-dimethylethyl)phenyl]-1H-indole-2-carboxylic acid;
1-({3-[(cyclopropylmethyl)oxy]-5-hydroxyphenyl}methyl)-3-[4-(1,1-dimethylethyl)phenyl]-1H-indole-2-carboxylic acid;
1-{[3-[(cyclopropylmethyl)oxy]-5-(methyloxy)phenyl]methyl}-3-[4-(1,1-dimethylethyl)phenyl]-1H-indole-2-carboxylic acid;
1-({3,5-bis[(cyclopropylmethyl)oxy]phenyl}methyl)-3-[4-(1,1-dimethylethyl)phenyl]-1H-indole-2-carboxylic acid;
1-({3-[(cyclopropylmethyl)oxy]-5-[(3-methylbutyl)oxy]phenyl}methyl)-3-[4-(1,1-dimethylethyl)phenyl]-1H-indole-2-carboxylic acid;
1-[(4′-carboxy-3-biphenylyl)methyl]-3-[4-(1,1-dimethylethyl)phenyl]-1H-indole-2-carboxylic acid;
3-[4-(1,1-dimethylethyl)phenyl]-1-({4′-[(phenylmethyl)oxy]-3-biphenylyl}methyl)-1H-indole-2-carboxylic acid;
3-[4-(1,1-dimethylethyl)phenyl]-1-[(4′-hydroxy-3-biphenylyl)methyl]-1H-indole-2-carboxylic acid;
3-[4-(1,1-dimethylethyl)phenyl]-1-[(4′-hydroxy-4-methyl-3-biphenylyl)methyl]-1H-indole-2-carboxylic acid;
1-[(4′-carboxy-4-methyl-3-biphenylyl)methyl]-3-[4-(1,1-dimethylethyl)phenyl]-1H-indole-2-carboxylic acid;
1-({4′-[(1-carboxy-1-methylethyl)oxy]-4-methyl-3-biphenylyl}methyl)-3-[4-(1,1-dimethylethyl)phenyl]-1H-indole-2-carboxylic acid;
3-[4-(1,1-dimethylethyl)phenyl]-1-{[4-methyl-4′-(methyloxy)-3-biphenylyl]methyl}-1H-indole-2-carboxylic acid;
3-(4-acetylphenyl)-1-[(4′-carboxy-4-methyl-3-biphenylyl)methyl]-1H-indole-2-carboxylic acid;
1-({4′-carboxy-5-[(cyclopropylmethyl)oxy]-3-biphenylyl}methyl)-3-[4-(1,1-dimethylethyl)phenyl]-1H-indole-2-carboxylic acid;
1-[(4′-hydroxy-3-biphenylyl)methyl]-3-[6-(methyloxy)-3-pyridinyl]-1H-indole-2-carboxylic acid;
3-[4-(1,1-dimethylethyl)phenyl]-1-{[4-methyl-3′-(methylthio)-3-biphenylyl]methyl}-1H-indole-2-carboxylic acid;
1-{[4′-carboxy-5-(methyloxy)-3-biphenylyl]methyl}-3-[4-(1,1-dimethylethyl)phenyl]-1H-indole-2-carboxylic acid;
1-({4′-carboxy-5-[(phenylmethyl)oxy]-3-biphenylyl}methyl)-3-[4-(1,1-dimethylethyl)phenyl]-1H-indole-2-carboxylic acid;
1-[(4′-carboxy-5-{[(methyloxy)methyl]oxy}-3-biphenylyl)methyl]-3-[4-(1,1-dimethylethyl)phenyl]-1H-indole-2-carboxylic acid;
1-[(4′-carboxy-4-methyl-3-biphenylyl)methyl]-3-[6-(methyloxy)-3-pyridinyl]-1H-indole-2-carboxylic acid;
1-[(4′-carboxy-5-hydroxy-3-biphenylyl)methyl]-3-[4-(1,1-dimethylethyl)phenyl]-1H-indole-2-carboxylic acid;
3-[4-(1,1-dimethylethyl)phenyl]-1-{[4′-(methylthio)-3-biphenylyl]methyl}-1H-indole-2-carboxylic acid;
3-[4-(1,1-dimethylethyl)phenyl]-1-{[4′-(methylsulfonyl)-3-biphenylyl]methyl}-1H-indole-2-carboxylic acid;
3-[4-(1,1-dimethylethyl)phenyl]-1-{[3′-(methylsulfonyl)-3-biphenylyl]methyl}-1H-indole-2-carboxylic acid;
3-[4-(1,1-dimethylethyl)phenyl]-1-{[3-(4-morpholinyl)phenyl]methyl}-1H-indole-2-carboxylic acid;
3-[4-(1,1-dimethylethyl)phenyl]-1-{[2-methyl-5-(4-morpholinyl)phenyl]methyl}-1H-indole-2-carboxylic acid;
1-({4′-[(dimethylamino)carbonyl]-4-methyl-3-biphenylyl}methyl)-3-[4-(1,1-dimethylethyl)phenyl]-1H-indole-2-carboxylic acid;
3-[4-(1,1-dimethylethyl)phenyl]-1-({4-methyl-3′-[(methylamino)carbonyl]-3-biphenylyl}methyl)-1H-indole-2-carboxylic acid;
3-[4-(1,1-dimethylethyl)phenyl]-1-[(4-methyl-3′-{[(2-thienylmethyl)amino]carbonyl}-3-biphenylyl)methyl]-1H-indole-2-carboxylic acid;
3-[4-(1,1-dimethylethyl)phenyl]-1-{[4-methyl-3′-({[2-(2-thienyl)ethyl]amino}carbonyl)-3-biphenylyl]methyl}-1H-indole-2-carboxylic acid;
3-[4-(1,1-dimethylethyl)phenyl]-1-[(4-methyl-4′-{[(2-thienylmethyl)amino]carbonyl}-3-biphenylyl)methyl]-1H-indole-2-carboxylic acid;
3-[4-(1,1-dimethylethyl)phenyl]-1-{[4-methyl-4′-({[2-(2-thienyl)ethyl]amino}carbonyl)-3-biphenylyl]methyl}-1H-indole-2-carboxylic acid;
3-[4-(1,1-dimethylethyl)phenyl]-1-({3-[4-(methylsulfonyl)-1-piperazinyl]phenyl}methyl)-1H-indole-2-carboxylic acid;
1-{[3-(4-acetyl-1-piperazinyl)phenyl]methyl}-3-[4-(1,1-dimethylethyl)phenyl]-1H-indole-2-carboxylic acid;
3-[4-(1,1-dimethylethyl)phenyl]-1-[(3-{4-[(methyloxy)carbonyl]-1-piperazinyl}phenyl)methyl]-1H-indole-2-carboxylic acid;
1-({3-[4-(aminocarbonyl)-1-piperazinyl]phenyl}methyl)-3-[4-(1,1-dimethylethyl)phenyl]-1H-indole-2-carboxylic acid;
1-[(3-{4-[({[(1,1-dimethylethyl)oxy]carbonyl}amino)sulfonyl]-1-piperazinyl}phenyl)methyl]-3-[4-(1,1-dimethylethyl)phenyl]-1H-indole-2-carboxylic acid;
1-({3-[4-(aminosulfonyl)-1-piperazinyl]phenyl}methyl)-3-[4-(1,1-dimethylethyl)phenyl]-1H-indole-2-carboxylic acid;
1-[(3-[(cyclopropylmethyl)oxy]-5-{[2-(dimethylamino)ethyl]oxy}phenyl)methyl]-3-[4-(1,1-dimethylethyl)phenyl]-1H-indole-2-carboxylic acid;
1-[(3-[(cyclopropylmethyl)oxy]-5-{[2-(1-pyrrolidinyl)ethyl]oxy}phenyl)methyl]-3-[4-(1,1-dimethylethyl)phenyl]-1H-indole-2-carboxylic acid;
1-[(3-[(cyclopropylmethyl)oxy]-5-{[2-(4-morpholinyl)ethyl]oxy}phenyl)methyl]-3-[4-(1,1-dimethylethyl)phenyl]-1H-indole-2-carboxylic acid;
1-[(3-[(cyclopropylmethyl)oxy]-5-{[3-(dimethylamino)propyl]oxy}phenyl)methyl]-3-[4-(1,1-dimethylethyl)phenyl]-1H-indole-2-carboxylic acid;
3-[4-(1,1-dimethylethyl)phenyl]-1-{[3-(4-thiomorpholinyl)phenyl]methyl}-1H-indole-2-carboxylic acid;
3-[4-(1,1-dimethylethyl)phenyl]-1-{[3-(1,1-dioxido-4-thiomorpholinyl)phenyl]methyl}-1H-indole-2-carboxylic acid;
3-[4-(1,1-dimethylethyl)phenyl]-1-[(3-{4-[(ethyloxy)carbonyl]-1-piperazinyl}phenyl)methyl]-1H-indole-2-carboxylic acid;
3-[4-(1,1-dimethylethyl)phenyl]-1-{[3-(4-{[(1-methylethyl)oxy]carbonyl}-1-piperazinyl)phenyl]methyl}-1H-indole-2-carboxylic acid;
3-[4-(1,1-dimethylethyl)phenyl]-1-({3-[4-({[2-(methyloxy)ethyl]oxy}carbonyl)-1-piperazinyl]phenyl}methyl)-1H-indole-2-carboxylic acid;
1-[(3-{[(dimethylamino)carbonyl]oxy}-5-{[2-(methyloxy)ethyl]oxy}phenyl)methyl]-3-[4-(1,1-dimethylethyl)phenyl]-1H-indole-2-carboxylic acid;
3-[4-(1,1-dimethylethyl)phenyl]-1-[(3-{[2-(methyloxy)ethyl]oxy}-5-{[(4-methyl-1-piperazinyl)carbonyl]oxy}phenyl)methyl]-1H-indole-2-carboxylic acid;
3-[4-(1,1-dimethylethyl)phenyl]-1-({3-{[2-(methyloxy)ethyl]oxy}-5-[(1-piperidinylcarbonyl)oxy]phenyl}methyl)-1H-indole-2-carboxylic acid;
3-[4-(1,1-dimethylethyl)phenyl]-1-({3-{[2-(methyloxy)ethyl]oxy}-5-[(4-morpholinylcarbonyl)oxy]phenyl}methyl)-1H-indole-2-carboxylic acid;
3-[4-(1,1-dimethylethyl)phenyl]-1-[(3-{[2-(methyloxy)ethyl]oxy}-5-{[(2-oxo-1-imidazolidinyl)carbonyl]oxy}phenyl)methyl]-1H-indole-2-carboxylic acid;
1-[(3-[(cyclopropylmethyl)oxy]-5-{[2-(1H-pyrrol-1-yl)ethyl]oxy}phenyl)methyl]-3-{[3-(trifluoromethyl)phenyl]methyl}-1H-indole-2-carboxylic acid;
1-({3-[(cyclopropylmethyl)oxy]-5-[(3-{[2-(methyloxy)ethyl]oxy}propyl)oxy]phenyl}methyl)-3-{[3-(trifluoromethyl)phenyl]methyl}-1H-indole-2-carboxylic acid;
1-[(3-[(cyclopropylmethyl)oxy]-5-{[2-(methyloxy)ethyl]oxy}phenyl)methyl]-3-{[3-(trifluoromethyl)phenyl]methyl}-1H-indole-2-carboxylic acid;
1-[(3-[(cyclopropylmethyl)oxy]-5-{[3-(dimethylamino)propyl]oxy}phenyl)methyl]-3-{[3-(trifluoromethyl)phenyl]methyl}-1H-indole-2-carboxylic acid hydrochloride;
1-[(3,5-bis{[2-(methyloxy)ethyl]oxy}phenyl)methyl]-3-{[3-(trifluoromethyl)phenyl]methyl}-1H-indole-2-carboxylic acid;
1-({3,5-bis[(cyclopropylmethyl)oxy]phenyl}methyl)-3-{[3-(trifluoromethyl)phenyl]methyl}-1H-indole-2-carboxylic acid;
3-(1-benzofuran-2-yl)-1-[(3,5-bis{[2-(methyloxy)ethyl]oxy}phenyl)methyl]-1H-indole-2-carboxylic acid;
1-[(3,5-bis{[2-(methyloxy)ethyl]oxy}phenyl)methyl]-3-{[4-(1,1-dimethylethyl)phenyl]oxy}-1H-indole-2-carboxylic acid;
1-[(3,5-bis{[2-(methyloxy)ethyl]oxy}phenyl)methyl]-3-{[4-(1,1-dimethylethyl)phenyl]amino}-1H-indole-2-carboxylic acid;
1-{[3,5-bis(trifluoromethyl)phenyl]methyl}-3-[4-(1,1-dimethylethyl)phenyl]-1H-indole-2-carboxylic acid;
3-[4-(1,1-dimethylethyl)phenyl]-1-{[3-{[2-(methyloxy)ethyl]oxy}-5-(trifluoromethyl)phenyl]methyl}-1H-indole-2-carboxylic acid;
1-{[3-[(cyclopropylmethyl)oxy]-5-(trifluoromethyl)phenyl]methyl}-3-[4-(1,1-dimethylethyl)phenyl]-1H-indole-2-carboxylic acid;
1-[(3,5-bis{[2-(methyloxy)ethyl]oxy}phenyl)methyl]-3-[4-(1,1-dimethylethyl)phenyl]-1H-indole-2-carboxylic acid;
3-[4-(1,1-dimethylethyl)phenyl]-1-({3-{[2-(methyloxy)ethyl]oxy}-5-[(phenylmethyl)oxy]phenyl}methyl)-1H-indole-2-carboxylic acid; and
3-[4-(1,1-dimethylethyl)phenyl]-1-{[3-{[2-(methyloxy)ethyl]oxy}-5-(4-morpholinyl)phenyl]methyl}-1H-indole-2-carboxylic acid.

While the embodiments or preferred groups for each variable have generally been listed above separately for each variable, compounds of this invention include those in which several of each variable in formulae I, II, III, IV or V, are selected from the embodiments or preferred groups for each variable. Therefore, this invention is intended to include all combinations of embodiments and preferred groups.

As used herein, the term “treatment” refers to alleviating the specified condition, eliminating or reducing the symptoms of the condition, slowing or eliminating the progression of the condition and preventing or delaying the initial occurrence of the condition in a subject, or reoccurrence of the condition in a previously afflicted subject.

One embodiment of the present invention is the use of the compounds of the present invention for the treatment of a variety of disorders including, but not limited to, type 2 diabetes mellitus; hyperglycemia; insulin resistance; chronic inflammation related disorders including but not limited to rheumatoid arthritis; inflammatory digestive diseases including but not limited to ulcerative colitis and Crohn's disease; fatty liver disease; psoriasis; dyslipidemia; hypercholesteremia; hypertriglyceridemia; syndrome X; hypertension; type I diabetes; polycystic ovary syndrome; Alzhiemers disease; cardiovascular disease including but not limited to vascular restenosis, atherosclerosis, and myocardial infarctions; other microvascular and macrovascular diseases including but not limited to retinopathy; obesity; anorexia bulimia; anorexia nervosa; cancer; and infertility.

In another embodiment, the compounds of the present invention are useful for the treatment or prevention of type II diabetes mellitus or syndrome X and are believed to cause less fluid accumulation and/or weight gain in patients that typically suffer from fluid accumulation and/or weight gain when treated with PPARγ agonists such as, for example, rosiglitazone, pioglitazone, or troglitazone.

The compounds of the present invention may crystallize in more than one form, a characteristic known as polymorphism, and such polymorphic forms (“polymorphs”) are within the scope of the present invention. Polymorphism generally may occur as a response to changes in temperature, pressure, or both. Polymorphism may also result from variations in the crystallization process. Polymorphs may be distinguished by various physical characteristics known in the art such as x-ray diffraction patterns, solubility, and melting point.

Certain of the compounds described herein contain one or more chiral centers, or may otherwise be capable of existing as multiple stereoisomers. The scope of the present invention includes mixtures of stereoisomers as well as purified enantiomers or enantiomerically/diastereomerically enriched mixtures. Also included within the scope of the invention are the individual isomers of the compounds represented by formulae I, II, III, IV and V, as well as any wholly or partially equilibrated mixtures thereof. The present invention also includes the individual isomers of the compounds represented by the formulas above as mixtures with isomers thereof in which one or more chiral centers are inverted.

Typically, but not absolutely, the salts of the present invention are pharmaceutically acceptable salts. Salts encompassed within the term “pharmaceutically acceptable salts” refer to non-toxic salts of the compounds of this invention. Salts of the compounds of the present invention may comprise acid addition salts. Representative salts include acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, laurate, malate, maleate, mandelate, mesylate, methylbromide, methylnitrate, methylsulfate, monopotassium maleate, mucate, napsylate, nitrate, N-methylglucamine, oxalate, pamoate (embonate), palmitate, pantothenate, phosphate/diphosphate, polygalacturonate, potassium, salicylate, sodium, stearate, subacetate, succinate, sulfate, tannate, tartrate, teoclate, tosylate, triethiodide, trimethylammonium, and valerate salts. Other salts, which are not pharmaceutically acceptable, may be useful in the preparation of compounds of this invention and these should be considered to form a further aspect of the invention.

As used herein, the term “solvate” refers to a complex of variable stoichiometry formed by a solute (in this invention, a compound of the present invention) and a solvent. Such solvents, for the purpose of the invention, should not interfere with the biological activity of the solute. Non-limiting examples of suitable solvents include, but are not limited to water, methanol, ethanol, and acetic acid. Preferably the solvent used is a pharmaceutically acceptable solvent. Non-limiting examples of suitable pharmaceutically acceptable solvents include water, ethanol, and acetic acid. Most preferably the solvent used is water.

As used herein, the term “physiologically functional derivative” refers to any pharmaceutically acceptable derivative of a compound of the present invention that, upon administration to a mammal, is capable of providing (directly or indirectly) a compound of the present invention or an active metabolite thereof. Such derivatives, for example, esters and amides, will be clear to those skilled in the art, without undue experimentation. Reference may be made to the teaching of Burger's Medicinal Chemistry And Drug Discovery, 5^{th} Edition, Vol 1: Principles and Practice, which is incorporated herein by reference to the extent that it teaches physiologically functional derivatives.

As used herein, the term “effective amount” means that amount of a drug or pharmaceutical agent that will elicit the biological or medical response of a tissue, system, animal, or human that is being sought, for instance, by a researcher or clinician. The biological or medical response may be considered a prophylactic response or a treatment response. The term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function. For use in therapy, therapeutically effective amounts of a compound of the present invention may be administered as the raw chemical. Additionally, the active ingredient may be presented as a pharmaceutical composition.

Accordingly, the invention further provides pharmaceutical compositions that include effective amounts of compounds of the present invention and one or more pharmaceutically acceptable carriers, diluents, or excipients. The compounds of the present invention are as herein described. The carrier(s), diluent(s) or excipient(s) must be acceptable, in the sense of being compatible with the other ingredients of the formulation and not deleterious to the recipient of the pharmaceutical composition.

In accordance with another aspect of the invention there is also provided a process for the preparation of a pharmaceutical formulation including admixing a compound of the present invention with one or more pharmaceutically acceptable carriers, diluents or excipients.

A therapeutically effective amount of a compound of the present invention will depend upon a number of factors. For example, the species, age, and weight of the recipient, the precise condition requiring treatment and its severity, the nature of the formulation, and the route of administration are all factors to be considered. The therapeutically effective amount ultimately should be at the discretion of the attendant physician or veterinarian. Regardless, an effective amount of a compound of the present invention for the treatment of humans suffering from type 2 diabetes mellitus, generally, should be in the range of 0.05 to 100 mg/kg body weight of recipient (mammal) per day. More usually the effective amount should be in the range of 0.1 to 10 mg/kg body weight per day. Thus, for a 70 kg adult mammal the actual amount per day would usually be from 7 to 700 mg. This amount may be given in a single dose per day or in a number (such as two, three, four, five, or more) of sub-doses per day such that the total daily dose is the same. An effective amount of a salt or solvate may be determined as a proportion of the effective amount of the compound of the present invention per se. Similar dosages should be appropriate for treatment of the other conditions referred to herein.

Pharmaceutical formulations may be presented in unit dose forms containing a predetermined amount of active ingredient per unit dose. Such a unit may contain, as a non-limiting example, 0.5 mg to 1 g of a compound of the present invention, depending on the condition being treated, the route of administration, and the age, weight, and condition of the patient. Preferred unit dosage formulations are those containing a daily dose or sub-dose, as herein above recited, or an appropriate fraction thereof, of an active ingredient. Such pharmaceutical formulations may be prepared by any of the methods well known in the pharmacy art.

Pharmaceutical formulations may be adapted for administration by any appropriate route, for example by an oral (including buccal or sublingual), rectal, nasal, topical (including buccal, sublingual or transdermal), vaginal, or parenteral (including subcutaneous, intramuscular, intravenous or intradermal) route. Such formulations may be prepared by any method known in the art of pharmacy, for example by bringing into association the active ingredient with the carrier(s) or excipient(s).

Pharmaceutical formulations adapted for oral administration may be presented as discrete units such as capsules or tablets; powders or granules; solutions or suspensions, each with aqueous or non-aqueous liquids; edible foams or whips; or oil-in-water liquid emulsions or water-in-oil liquid emulsions. For instance, for oral administration in the form of a tablet or capsule, the active drug component may be combined with an oral, non-toxic pharmaceutically acceptable inert carrier such as ethanol, glycerol, water, and the like. Generally, powders are prepared by comminuting the compound to a suitable fine size and mixing with an appropriate pharmaceutical carrier such as an edible carbohydrate, as, for example, starch or mannitol. Flavorings, preservatives, dispersing agents, and coloring agents may also be present.

Capsules are made by preparing a powder, liquid, or suspension mixture and encapsulating with gelatin or some other appropriate shell material. Glidants and lubricants such as colloidal silica, talc, magnesium stearate, calcium stearate, or solid polyethylene glycol may be added to the mixture before the encapsulation. A disintegrating or solubilizing agent such as agar-agar, calcium carbonate or sodium carbonate may also be added to improve the availability of the medicament when the capsule is ingested. Moreover, when desired or necessary, suitable binders, lubricants, disintegrating agents, and coloring agents may also be incorporated into the mixture. Examples of suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants useful in these dosage forms include, for example, sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

Tablets may be formulated, for example, by preparing a powder mixture, granulating or slugging, adding a lubricant and disintegrant, and pressing into tablets. A powder mixture may be prepared by mixing the compound, suitably comminuted, with a diluent or base as described above. Optional ingredients include binders such as carboxymethylcellulose, aliginates, gelatins, or polyvinyl pyrrolidone, solution retardants such as paraffin, resorption accelerators such as a quaternary salt, and/or absorption agents such as bentonite, kaolin, or dicalcium phosphate. The powder mixture may be wet-granulated with a binder such as syrup, starch paste, acadia mucilage or solutions of cellulosic or polymeric materials, and forcing through a screen. As an alternative to granulating, the powder mixture may be run through the tablet machine and the result is imperfectly formed slugs broken into granules. The granules may be lubricated to prevent sticking to the tablet forming dies by means of the addition of stearic acid, a stearate salt, talc or mineral oil. The lubricated mixture is then compressed into tablets. The compounds of the present invention may also be combined with a free flowing inert carrier and compressed into tablets directly without going through the granulating or slugging steps. A clear or opaque protective coating consisting of a sealing coat of shellac, a coating of sugar or polymeric material, and a polish coating of wax may be provided. Dyestuffs may be added to these coatings to distinguish different unit dosages.

Oral fluids such as solutions, syrups, and elixirs may be prepared in dosage unit form so that a given quantity contains a predetermined amount of the compound. Syrups may be prepared, for example, by dissolving the compound in a suitably flavored aqueous solution, while elixirs may be prepared through the use of a non-toxic alcoholic vehicle. Suspensions may be formulated generally by dispersing the compound in a non-toxic vehicle. Solubilizers and emulsifiers such as ethoxylated isostearyl alcohols and polyoxy ethylene sorbitol ethers, preservatives; flavor additives such as peppermint oil, or natural sweeteners, saccharin, or other artificial sweeteners; and the like may also be added.

Where appropriate, dosage unit formulations for oral administration may be microencapsulated. The formulation may also be prepared to prolong or sustain the release as for example by coating or embedding particulate material in polymers, wax or the like.

The compounds of the present invention may also be administered in the form of liposome delivery systems, such as small unilamellar vesicles, large unilamellar vesicles, and multilamellar vesicles. Liposomes may be formed from a variety of phospholipids, such as cholesterol, stearylamine, or phosphatidylcholines.

The compounds of the present invention may also be delivered by the use of monoclonal antibodies as individual carriers to which the compound molecules are coupled.

The compounds may also be coupled with soluble polymers as targetable drug carriers. Such polymers may include polyvinylpyrrolidone (PVP), pyran copolymer, polyhydroxypropylmethacrylamide-phenol, polyhydroxyethyl-aspartamidephenol, or polyethyleneoxidepolylysine substituted with palmitoyl residues. Furthermore, the compounds may be coupled to a class of biodegradable polymers useful in achieving controlled release of a drug; for example, polylactic acid, polyepsilon caprolactone, polyhydroxy butyric acid, polyorthoesters, polyacetals, polydihydropyrans, polycyanoacrylates, and cross-linked or amphipathic block copolymers of hydrogels.

Pharmaceutical formulations adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6), 318 (1986), incorporated herein by reference as related to such delivery systems.

Pharmaceutical formulations adapted for topical administration may be formulated as ointments, creams, suspensions, lotions, powders, solutions, pastes, gels, sprays, aerosols, or oils.

For treatments of the eye or other external tissues, for example mouth and skin, the formulations may be applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical formulations adapted for topical administrations to the eye include eye drops wherein the active ingredient is dissolved or suspended in a suitable carrier, especially an aqueous solvent.

Pharmaceutical formulations adapted for topical administration in the mouth include lozenges, pastilles, and mouthwashes.

Pharmaceutical formulations adapted for nasal administration, where the carrier is a solid, include a coarse powder having a particle size for example in the range 20 to 500 microns. The powder is administered in the manner in which snuff is taken, i.e., by rapid inhalation through the nasal passage from a container of the powder held close up to the nose. Suitable formulations wherein the carrier is a liquid, for administration as a nasal spray or as nasal drops, include aqueous or oil solutions of the active ingredient.

Pharmaceutical formulations adapted for administration by inhalation include fine particle dusts or mists, which may be generated by means of various types of metered dose pressurized aerosols, nebulizers, or insufflators.

Pharmaceutical formulations adapted for rectal administration may be presented as suppositories or as enemas.

Pharmaceutical formulations adapted for vaginal administration may be presented as pessaries, tampons, creams, gels, pastes, foams, or spray formulations.

Pharmaceutical formulations adapted for parenteral administration include aqueous and non-aqueous sterile injection solutions which may contain anti-oxidants, buffers, bacteriostats, and solutes that render the formulation isotonic with the blood of the intended recipient; and aqueous and non-aqueous sterile suspensions which may include suspending agents and thickening agents. The formulations may be presented in unit-dose or multi-dose containers, for example sealed ampules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules, and tablets.

In addition to the ingredients particularly mentioned above, the formulations may include other agents conventional in the art having regard to the type of formulation in question. For example, formulations suitable for oral administration may include flavoring or coloring agents.

The compounds of the present invention and their salts or solvates thereof, may be employed alone or in combination with other therapeutic agents for the treatment of the above-mentioned conditions. For example, for the treatment of type 2 diabetes, a compound of the present invention may be administered in combination with one or more anti-diabetic agents such as sulfonylureas, meglitinides, biguanides such as metformin, thiazolidinediones, alpha-glucosidase inhibitors such as acarbose and meglitol, amylin, and insulin and insulin mimetics. The compound(s) of the present invention and the other pharmaceutically active agent(s) may be administered together or separately and, when administered separately, administration may occur simultaneously or sequentially, in any order. The amounts of the compound(s) of the present invention and the other pharmaceutically active agent(s) and the relative timings of administration will be selected in order to achieve the desired combined therapeutic effect. The administration of a combination of a compound of the present invention with other treatment agents may be by concomitant administration in: (1) a unitary pharmaceutical composition including all compounds; or (2) separate pharmaceutical compositions each including one of the compounds. Alternatively, the combination may be administered separately in a sequential manner wherein one treatment agent is administered first and the other second or vice versa. Such sequential administration may be close in time or remote in time. The route of administration for each of the compounds may be the same as the others, or different.

The compounds of the present invention may be used in the treatment of a variety of disorders and conditions and, as such, the compounds of the present invention may be used in combination with a variety of other suitable therapeutic agents useful in the treatment of those disorders or conditions. Non-limiting examples include combinations of the present invention with other compounds of the present invention and anti-diabetic agents, anti-osteoporosis agents, anti-obesity agents, anti-inflammatory agents, anti-anxiety agents, anti-depressants, anti-hypertensive agents, anti-platelet agents, anti-thrombotic and thrombolytic agents, cardiac glycosides, cholesterol or lipid lowering agents, mineralocorticoid receptor antagonists, phosphodiesterase inhibitors, kinase inhibitors, thyroid mimetics, anabolic agents, viral therapies, cognitive disorder therapies, sleeping disorder therapies, sexual dysfunction therapies, contraceptives, cytotoxic agents, radiation therapy, anti-proliferative agents, and anti-tumor agents. Additionally, the compounds of the present invention may be combined with nutritional supplements such as amino acids, triglycerides, vitamins, minerals, creatine, piloic acid, carnitine, or coenzyme Q10.

The compounds of the present invention are believed useful, either alone or in combination with other agents, for the treatment of a variety of disorders including, but not limited to, type 2 diabetes mellitus; hyperglycemia; insulin resistance; chronic inflammation related disorders including but not limited to rheumatoid arthritis; inflammatory digestive diseases including but not limited to ulcerative colitis and Crohn's disease; fatty liver disease; psoriasis; dyslipidemia; hypercholesteremia; hypertriglyceridemia; syndrome X; hypertension; type I diabetes; polycystic ovary syndrome; Alzhiemers disease; cardiovascular disease including but not limited to vascular restenosis, atherosclerosis, and myocardial infarctions; other microvascular and macrovascular diseases including but not limited to retinopathy; obesity; anorexia bulimia; anorexia nervosa; cancer; and infertility. In one embodiment of the present invention is the use of the compounds of the present invention in combination with other pharmaceutically active agents for the treatment of hyperglycemia, type 2 diabetes, impaired glucose tolerance, insulin resistance, syndrome X, and dyslipidemia.

The compounds of this invention may be made by a variety of methods, including well-known standard synthetic methods. Illustrative general synthetic methods are set out below and then specific compounds of the invention are illustrated in the working Examples.

In all of the schemes described below, protecting groups for sensitive or reactive groups are employed where necessary in accordance with general principles of synthetic chemistry. Protecting groups are manipulated according to standard methods of organic synthesis (T. W. Green and P. G. M. Wuts (1991) Protecting Groups in Organic Synthesis, John Wiley & Sons, incorporated by reference with regard to protecting groups). These groups are removed at a convenient stage of the compound synthesis using methods that are readily apparent to those skilled in the art. The selection of processes as well as the reaction conditions and order of their execution shall be consistent with the preparation of compounds of the present invention.

Those skilled in the art will recognize if a stereocenter exists in compounds of the present invention. Accordingly, the present invention includes all possible stereoisomers and includes not only racemic compounds but the individual enantiomers as well. When a compound is desired as a single enantiomer, such may be obtained by stereospecific synthesis or by resolution of the final product or any convenient intermediate. Resolution of the final product, an intermediate, or a starting material may be effected by any suitable method known in the art. See, for example, Stereochemistry of Organic Compounds by E. L. Eliel, S. H. Wilen, and L. N. Mander (Wiley-Interscience, 1994), incorporated by reference with regard to stereochemistry.

### Abbreviations

As used herein the symbols and conventions used in these processes, schemes and examples are consistent with those used in the contemporary scientific literature, for example, the Journal of the American Chemical Society or the Journal of Biological Chemistry. Specifically, the following abbreviations may be used in the examples and throughout the specification:

Unless otherwise indicated, all temperatures are expressed in ° C. (degrees Centigrade). All reactions conducted under an inert atmosphere at room temperature unless otherwise noted. Reagents employed without synthetic details are commercially available or made according to literature procedures.

¹H NMR spectra were recorded on a Varian Unity-300, or a Varian Unity-400 instrument. Chemical shifts are expressed in parts per million (ppm, δ units). Coupling constants are in units of hertz (Hz). Splitting patterns describe apparent multiplicities and are designated as s (singlet), d (doublet), t (triplet), q (quartet), m (multiplet), or b (broad).

| | |
|---|---|
| DMF - Dimethylformamide | AlCl₃ - Aluminum Chloride |
| BnBr - Benzyl bromide | Br₂ - Bromine |
| Et₃N - Triethylamine | Pd(PPh₃)₄ - |
| P - Protecting Group | Tetrakistriphenylphosphine |
| L - Leaving Group | Palladium |
| KOH - Potassium Hydroxide | MnO₂ - Manganese Dioxide |
| EtOH - Ethanol | DCE - Dichloroethane |
| H₂O - water | Tf₂O - |
| K₂CO₃ - Potassium Carbonate | Trifluoromethanesulsonic |
| Pd/C - Palladium on Carbon | Anhydride |
| THF - Tetrahydrofuran | TEA - Triethylamine |
| KOH - Potassium Hydroxide | NMO - |
| EtOAc - Ethyl Acetate | N-Methylmorpholine-N-Oxide |
| ° C. - Degrees Celcius | OsO₄ - Osmium Tetroxide |
| CHCl₃ - Chloroform | NMP - 1-Methyl-2-Pyrrolidinone |
| DCM - Dichloromethane | DMPU - 1,3-Dimethylpyrimidinone |
| TFA - Trifluoroacetic Acid | EDCl•HCl - |
| DME - Dimethoxyethane | Ethylenediaminecarbodiimide |
| Na₂CO₃ - Sodium Carbonate | hyddrochloride |
| NaHCO₃ - Sodium Hydrogen | DMAP - Dimethylaminopyridine |
| Carbonate | TBAF - Tetrabutylammonium |
| Cs₂CO₃ - Cesium Carbonate | Fluoride |
| MsCl - Methanesulfonyl Chloride | DIAD - |
| NaOH - Sodium Hydroxide | Diisopropylazodicacarboxylate |
| H₂ - Hydrogen Gas | PPh₃ - Triphenylphosphine |
| MeOH - Methanol | KOtBu - Potassium Tertbutoxoxide |
| SOCl₂ - Thionyl Chloride | DIEA - Diisopropylethylamine |
| NaBH₄ - Sodium Borohydride | KCl - Potassium Chloride |
| CuO - Cuprous Oxide | LAH - Lithium Aluminum Hydride |
| CuNO₃•3H₂O = cupric | CuI - Cuprous Iodide |
| nitrate trihydrate | Et₂NH - Diethylamine |
| Na₂SO₄ - Sodium Sulfate | TFAA - Trifluoroacetic Anhydride |
| Pd(OAc)₂ - Palladium Diacetate | DMSO - Dimethylsulfoxide |
| P(t-butyl)₃ - Tri- | LiBr = Lithium Bromide |
| tertbutylphosphine | NaNO₂ = Sodium nitrite |
| Sat. = Saturated | AcOH = Acetic Acid |
| Aq = aqueous | (Rh(OAc)₂)₂ = rhodium II |
| NHCl₄ = Ammonium Chloride | acetate dimer |
| CH₃CN = Acetonitrile | NaHMDS = Sodium |
| CH₃I = Iodomethane | bis(trimethylsilyl)amide |
| n-BuLi = n-Butyllithium | H₂SO₄ = Sulfuric Acid |
| B(OiPr)₃ = Triisopropylborate | TBME = Tert-butyl |
| MgSO4 = Magnesium Sulfate | methyl ether |
| DMA = Dimethylacetamide | MTBE = Tert-butyl |
| | methyl ether |
| | HOBt = Hydroxybenzotriazole |
| | AcOH = Acetic Acid |
| | KNCO = potassium isocyanate |
| | tBuOH = t-butyl alcohol |
| | Na₂S₂O₃ = Sodium Thiosulfate |
| | DMA = Dimethylacetamide |

Compounds of the present invention may be made by the following routes depicted in SCHEMES 1-10:

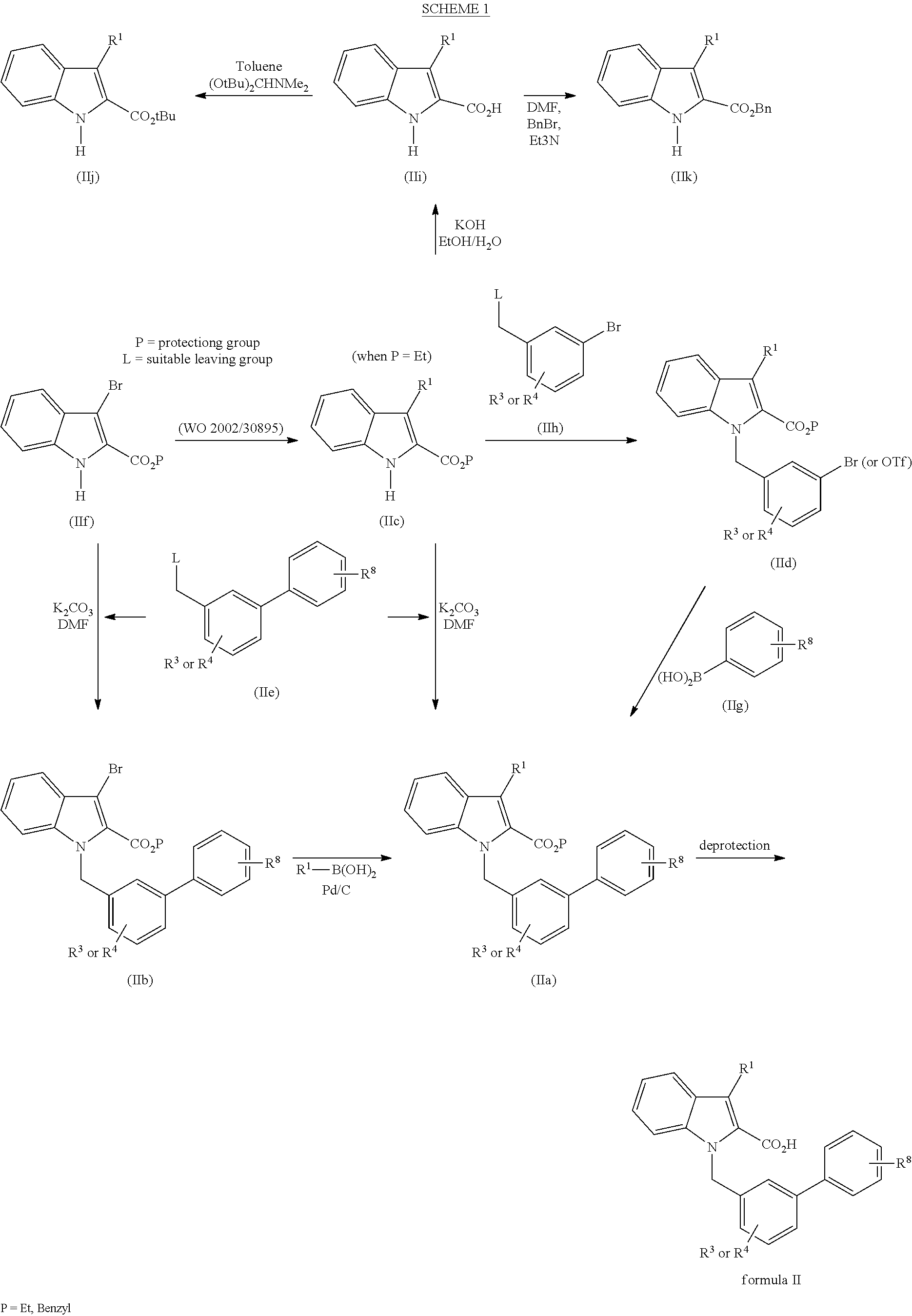

Compounds of formula II may be prepared from compounds of formula IIa by the deprotection of a protected acid. For methyl or ethyl esters of formula IIa, hydrolysis of these esters may be effected to afford compounds of formula II in an a polar solvent such as EtOH or THF in the presence of water and hydroxide ion, typically from an alkali metal hydroxide such as KOH or NaOH, at temperatures from 20° C. to 150° C. When P in formula IIa is a benzyl protecting group, deprotection of a benzyl ester of formula IIa to give compounds of formula II may be achieved by hydrogenolysis in a polar protic or nonprotic solvent such as EtOH, EtOAc or a polar halogenated solvent such as CHCl₃ at temperatures from 0° C. to 100° C. typically 23° C. in the presence of a catalyst such as Pd/C under an atmosphere of hydrogen gas. When P is a tert-butyl ester in formula IIa, compounds of formula II may be prepared from compounds of formula IIa in a polar halogenated solvent such as DCM in the presence of a strong acid such as TFA at temperatures from −20° C. to 50° C. typically 0° C. to 23° C. Compounds of formula IIa may be prepared from compounds of formula IIb by a Suzuki coupling with a boronic acid of formula R¹—B(OH)₂ in a polar aprotic solvent such as DME and water mixture with a palladium catalyst such as palladium tetrakistriphenylphosphine and a base such as Na₂CO₃ at temperatures from 23° C. to 150° C. such as 80° C. or the Suzuki coupling can be effected in a polar aprotic solvent such as DMF with palladium on carbon as catalyst with a base such as NaHCO₃ at elevated temperatures from 23° C. to 150° C. such as 90° C. Compounds of formula IIb may be prepared from compounds of formula IIf by alkylation with compounds of formula IIe in a polar aprotic solvent such as DMF at temperatures from 0° C. to 150° C. such as 80° C. in the presence of a base such as K₂CO₃. Compounds of formula IIf are known compounds or may be readily prepared by one skilled in the art. Compounds of formula IIe may be prepared as described in SCHEME 3 or SCHEME 4. Compounds of formula IIa may also be prepared from compounds of formula IIc by alkylation with compounds of formula IIe in a polar aprotic solvent such as DMF at temperatures from 0° C. to 150° C. such as 80° C. in the presence of a base such as K₂CO₃. Compounds of formula IIc have been reported (WO2002/30895). Compounds of formula IIa may also be prepared from compounds of formula IId by Suzuki coupling with compounds of formula IIg under typical Suzuki coupling conditions (palladium on carbon or palladium tetrakistriphenylphosphine as catalyst) in DMF and water solvent with a base such as NaHCO₃ or Na₂CO₃ at temperatures from 0° C. to 150° C. such as 90° C. Compounds of formula IIg are commercially available or may be readily prepared by one skilled in the art. Compounds of formula IId may be prepared by alkylation of compounds of formula IIc with compounds of formula IIh (L is a suitable leaving group such as bromide, chloride, or mesylate) in a polar aprotic solvent such as DMF at temperatures from 0° C. to 150° C. such as 80° C. in the presence of a base such as K₂CO₃. Compounds of formula IIh are known compounds or may be readily prepared by one skilled in the art. Certain compounds of formula IIh may be prepared as described in SCHEME 3 to give compounds of formula IIh′. Differentially protected compounds of formulas IIj and IIk may be prepared from compounds of formula IIc (P is ethyl) by first generating free acid intermediate compound of formula IIi in the presence of KOH in water and a polar protic solvent such as EtOH at temperatures from 0° C. to 150° C. such as 50° C. Compounds of formula IIk may then be prepared by alkylation of IIi with benzyl bromide in a polar aprotic solvent such as DMF with a base such as Et₃N. A tert-butyl ester of formula IIj may also be prepared from an acid of formula IIj in a nonpolar higher boiling solvent such toluene in the presence of the di-tertbutylacetal analog of DMF.

Certain compounds of formula II may also be prepared according to SCHEME 2

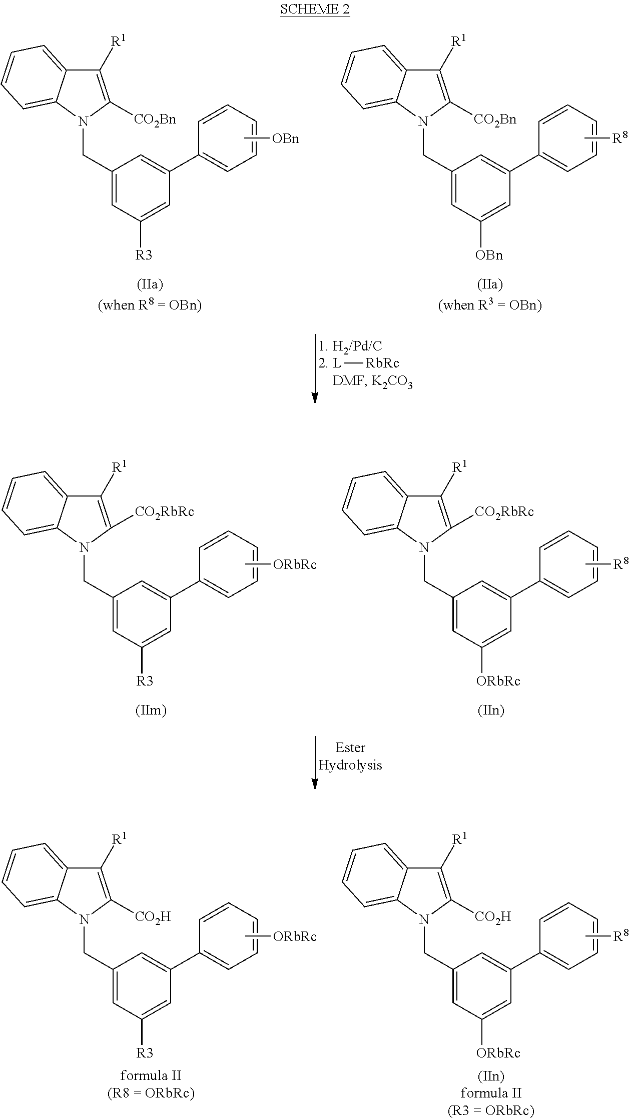

When R⁸ in formula IIa is a benzyl protected phenol, compounds of formula IIm may be prepared from compounds of formula IIa in the presence of a palladium catalyst such as palladium on carbon in a polar solvent such as a CHCl₃/MeOH mixture under an atmosphere of hydrogen from 1-60 psi at temperatures from 0° C. to 100° C., typically 23° C. Phenol intermediates of formula IIm may then be alkylated) in a polar aprotic solvent such as DMF at temperatures from 0° C. to 150° C. such as 80° C. in the presence of a base such as K₂CO₃ with a suitable alkylating reagent R^{c}R^{b}-L (L is a suitable leaving group) to generate ether compounds of formula IIa (R⁸═OR^{b}R^{c}). Likewise, when R³ in formula IIa is a benzyl protected phenol, compounds of formula IIn may be prepared from compounds of formula IIa in the presence of a palladium catalyst such as Pd/C in a polar solvent such as a CHCl₃/MeOH mixture under an atmosphere of hydrogen from 1-60 psi at temperatures from 0° C. to 100° C., typically 23° C. Phenol intermediates of formula IIn may then be alkylated in a polar aprotic solvent such as DMF at temperatures from 0° C. to 150° C. such as 80° C. in the presence of a base such as K₂CO₃ with a suitable alkylating reagent R^{c}R^{b}-L (L is a suitable leaving group) to generate ether compounds of formula IIa (R³═OR^{b}R^{c}).

Certain compounds of formula IIe may be prepared as shown in SCHEME 3.

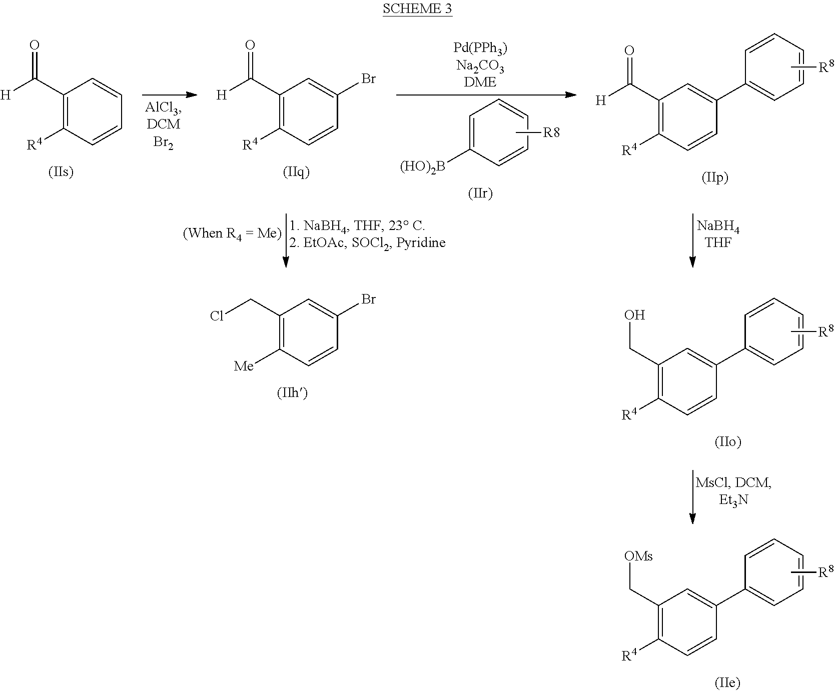

Compounds of formula IIe may be prepared from compounds of formula IIo in a polar halogenated solvent such as DCM in the presence of MsCl and a base such Et₃N at temperatures from −20° C. to 100° C. such as 0° C. to 23° C. Compounds of formula IIo may be prepared from compounds of formula IIp in a polar aprotic solvent such as THF in the presence of a reducing agent such as NaBH₄ at temperatures from −20° C. to 50° C. such as 0° C. Compounds of formula IIp may be prepared from compounds of formula IIq via Suzuki coupling with a compound of formula IIr in a polar aprotic solvent such as DME in the presence of a base such as Na₂CO₃ and in the presence of a palladium catalyst such as palladium tetrakistriphenylphosphorane at temperatures from 20° C. to 150° C. such as 80° C. Compounds of formula IIr are known or may be readily prepared by one skilled in the art. Compounds of formula IIq may be prepared by bromination of compounds of formula IIs in a halogenated solvent such as DCM in the presence of bromine and AlCl₃ at temperatures from −78° C. to 23° C. such as 0° C. Compounds of formula IIs are known or may be readily prepared by one skilled in the art.

Certain compounds of formula IIe may also be prepared as shown in SCHEME 4

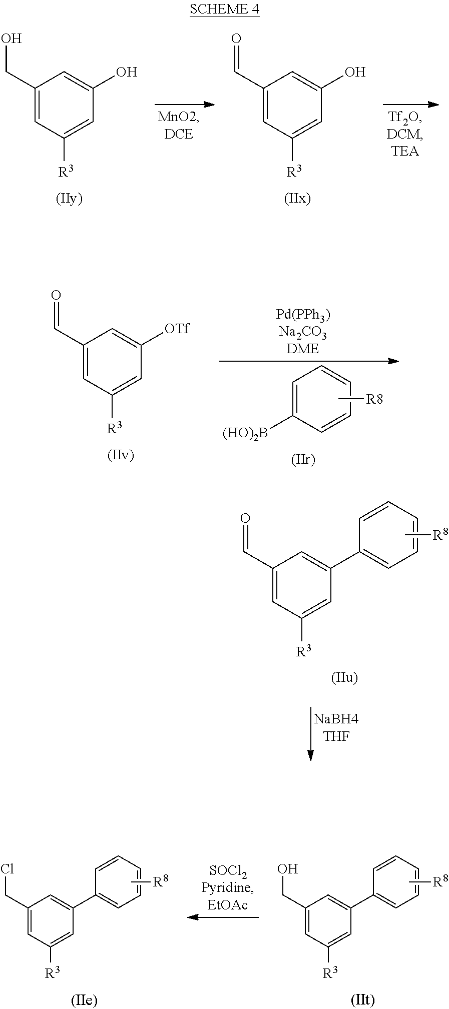

Certain compounds of formula IIe may be prepared from compounds of formula IIt in a polar aprotic solvent such as EtOAc with thionyl chloride in the presence of a base such as pyridine at temperatures from −20° C. to 100° C. such as 0° C. Compounds of formula IIt may be prepared from compounds of formula IIu in a polar aprotic solvent such as THF in the presence of a reducing agent such as NaBH₄ at temperatures from −20° C. to 50° C. such as 0° C. Compounds of formula IIu may be prepared from compounds of formula IIv via Suzuki coupling with a compound of formula IIr in a polar aprotic solvent such as DME in the presence of a base such as Na₂CO₃ and in the presence of a palladium catalyst such as palladium tetrakistriphenylphosphine at temperatures from 20° C. to 150° C. such as 80° C. Compounds of formula IIr are known or may be readily prepared by one skilled in the art. Compounds of formula IIv may be prepared from compounds of formula IIx in a polar halogenated solvent such as DCM with trifluoromethanesulfonic anhydride in the presence of a base such as Et₃N at temperatures from −78° C. to 50° C. such as 0° C. Compounds of formula IIx may be prepared via oxidation of compounds of formula IIy with an oxidant such as manganese dioxide in a halogenated solvent such as DCE at temperatures from 0° C. to 80° C. such as 23° C. Compounds of formula IIy are known or may be readily prepared by one skilled in the art (see for example SCHEME 7b).

Compounds of formula III may be prepared as shown in SCHEME 5

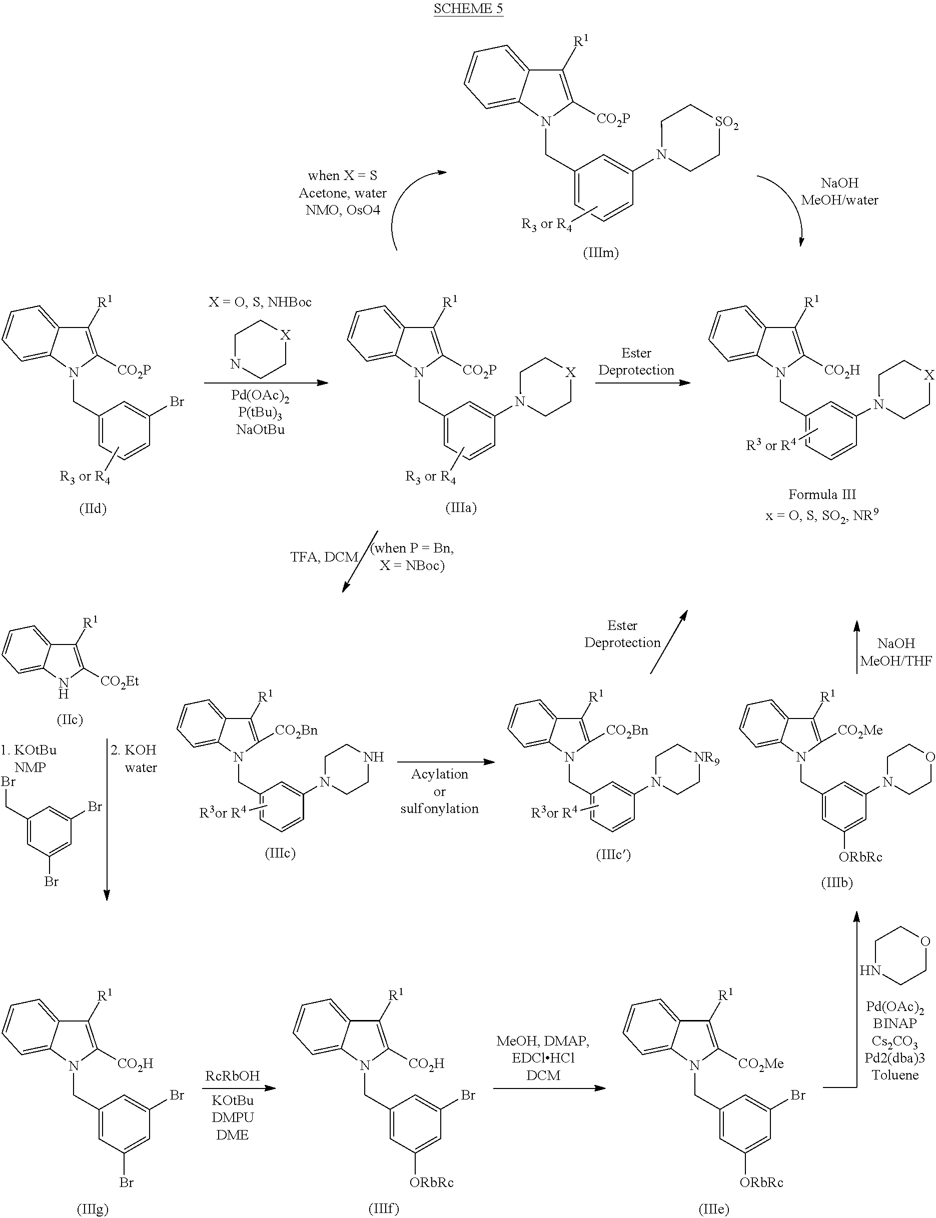

Certain compounds of formula III (X═O,S) may be prepared from compounds of formula IIIa in a polar solvent such EtOH and/or THF with aqueous hydroxide such as NaOH in water at temperatures from 23° C. to 100° C. such as 50° C. Compounds of formula IIIa may be prepared from aryl bromide compounds of formula IId via a metal mediated coupling with an amine in an aprotic solvent such as toluene in the presence of a ligand such as tri-(tertbutyl)phosphine, a base such as NaOtBu, and a catalytic quantity of a metal catalyst such as palladium diacetate at temperatures from 23° C. to 100° C. such as 50° C. Compounds of formula IId may be prepared as described in SCHEME 1. When X in formula IIIa is NBoc, compounds of formula IIIa may be converted to compounds of formula IIIc via acid catalyzed removal of the piperizine Boc protecting group in a polar solvent such as DCM in the presence of trifluoroacetic acid at temperatures from −20° C. to 50° C. such as 23° C. Amide, sulfonamide, urea, carbamate, and sulfamate compounds of formula IIIc′ may then be generated from compounds of formula IIIc via known acylation and sulfonylation conditions of the piperizine nitrogen group by methods known by one skilled in the art.

Compounds of formula III may also be prepared from compounds of formula IIIb in a polar solvent such MeOH and/or THF with aqueous hydroxide such as NaOH in water at temperatures from 23° C. to 100° C. such as 50° C. Compounds of formula IIIb may be prepared from a metal mediated coupling of aryl bromide compounds of formula IIIe with an amine such as morpholine in an aprotic solvent such as toluene in the presence of a ligand such as BINAP, a base such as Cs₂CO₃, and a palladium catalyst such as a mixture of palladium diacetate and Pd₂(dba)₃ at temperatures from 23° C. to 150° C. such as 50° C. Compounds of formula IIIe may be prepared from the esterification of compounds of formula IIIf in a polar protic solvent such as MeOH and a polar halogenated solvent such as DCM with a base such as DMAP in the presence of EDCI.HCl. Compounds of formula IIIf may be prepared from compounds of formula IIIg and a suitable alcohol R^{c}R^{b}OH in a polar solvent such as DME in the presence of DMPU and a strong base such as KOtBu at temperatures from 0° C. to 150° C. such as 35° C. to 115° C. Compounds of formula IIIg may be prepared by alkylation of compounds of formula IIc with 3,5-dibromobenzyl bromide in a polar solvent such as NMP in the presence of a strong base such as KOtBu at temperatures from 0° C. to 150° C. such as 23° C. to 50° C. followed by hydrolysis of the resulting intermediate ester by the addition of an aqueous solution of hydroxide such as from KOH at temperatures from 23° C. to 100° C. such as 60° C. Compounds of formula IIc are known or may be readily prepared by one skilled in the art. Certain compounds of formula III (X═SO₂) may be prepared from compounds of formula IIIm in a polar solvent such MeOH with aqueous hydroxide such as NaOH in water at temperatures from 0° C. to 100° C. Compounds of formula IIIm may be prepared from compounds of formula IIIa (X═S) in acetone and water with NMO and OsO₄ as oxidant.

Certain compounds of formula IIIa from SCHEME 5 may be prepared as shown in SCHEME 6

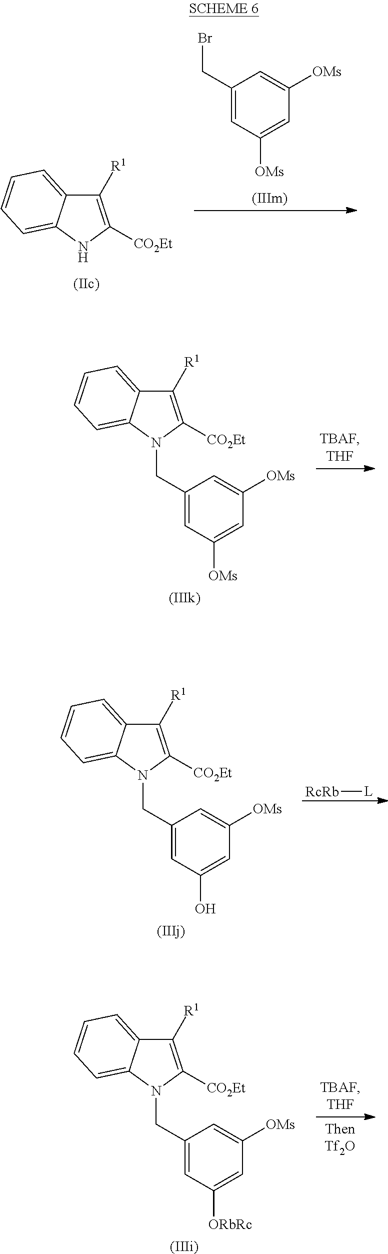

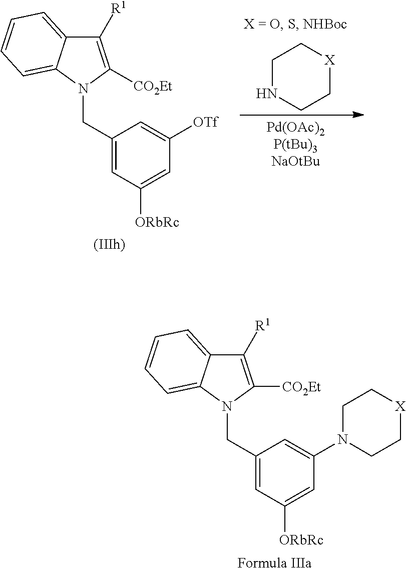

Compounds of formula IIIa (X═O, S, NHBoc) may be prepared from compounds of formula IIIh with a palladium assisted amination reaction utilizing a palladium catalyst such as palladium acetate and a phosphine ligand such as tri-(tertbutyl)phosphine in a polar aprotic solvent such as DME in the presence of a base such as NaOtBu at temperatures from 0° C. to 150° C. such as 80° C. Compounds of formula IIIh may be prepared from mono-mesylate compounds of formula IIIi by first hydrolyzing the mesylate in a polar solvent such as THF in the presence of TBAF and taking the resulting phenol intermediate and triflating with trifluoromethanesulfonyl anhydride in a polar halogenated solvent such as DCM at −20° C. to 60° C. Compounds of formula IIIi may be prepared by the alkylation of phenol compounds of formula IIIj in a polar aprotic solvent such as DMF in the presence of an alkylating reagent such as R^{c}R^{b}-L where L is a suitable leaving group at temperatures from 0° C. to 150° C. such as 80° C.

Compounds of formula IIIj may be prepared from compounds of formula IIIk in a polar solvent such as THF in the presence of TBAF at temperatures of 0° C. to 100° C. such as 70° C. Compounds of formula IIIk may be prepared by the alkylation of compounds of formula IIc with benzyl bromide compound of formula IIIm. Compound IIIm may be prepared from 3,5-dihydroxybenzyl alcohol by mesylation followed by bromination by standard methods by one skilled in the art.

Compounds of formula IV may be prepared from several different routes as shown in SCHEME 7a

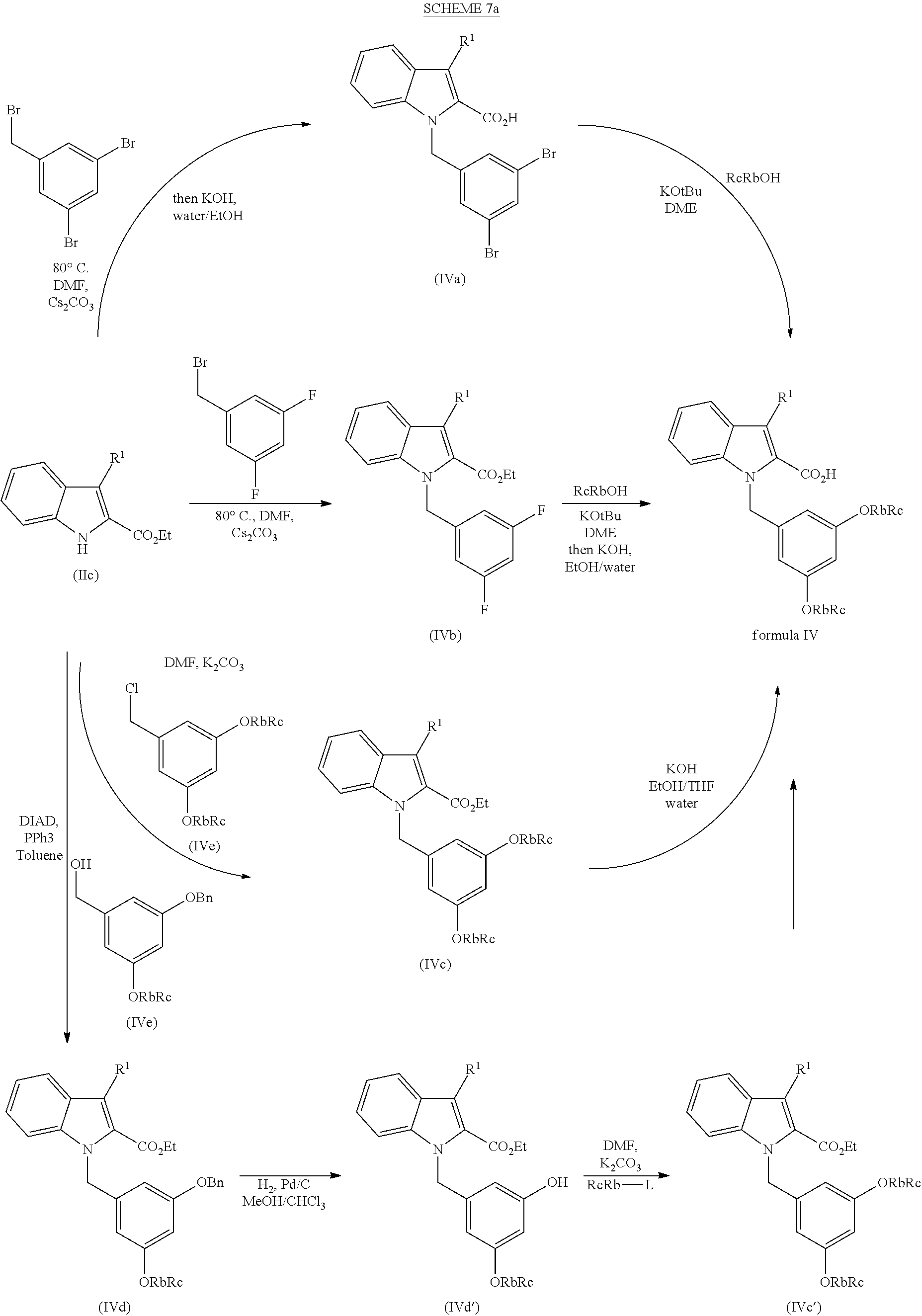

Compounds of formula IV may be prepared from dibromide compounds of formula IVa in a polar aprotic solvent such as DME in the presence of an alcohol R₈OH and a base such as KOtBu at temperatures from 0° C. to 150° C. such as 80° C. Compounds of formula IVa may be prepared from compounds of formula IIc via alkylation with 3,5-dibromobenzyl bromide in a polar aprotic solvent such as DMF with a base such as Cs₂CO₃ at temperatures from 23° C. to 150° C. such as 80° C. Compounds of formula IV may be prepared from diflouro compounds of formula IVb in a polar aprotic solvent such as DME in the presence of an alcohol R₈OH and a base such as KOtBu at temperatures from 0° C. to 150° C. such as 80° C. Compounds of formula IVb may be prepared from compounds of formula IIc via alkylation with 3,5-difluorobenzyl bromide in a polar aprotic solvent such as DMF with a base such as Cs₂CO₃ at temperatures from 23° C. to 150° C. such as 80° C. Compounds of formula IIc are known or may be readily prepared by one skilled in the art. Compounds of formula IV may be prepared from compounds of formula IVc in a polar aprotic and polar protic mixture of solvents such as EtOH and THF in the presence of water and hydroxide ion such as with KOH at temperatures from 0° C. to 100° C. such as 50° C. Compounds of formula IVc may be prepared from compounds of formula IIc in a polar aprotic solvent such as DMF with an alkylating reagent such as a compound with structural formula IVe with a base such as K₂CO₃ at temperatures from 0° C. to 150° C. such as 90° C. Compounds of formula IVe may be prepared as described in SCHEME 7b. Certain compounds of formula IV may be prepared from a deprotection/re-alkylation strategy. Compounds of formula IV may be prepared from compounds of formula IVc′ in a polar aprotic and polar protic mixture of solvents such as ethanol and THF in the presence of water and hydroxide ion such as with KOH at temperatures from 0° C. to 100° C. such as 50° C. Compounds of formula IVc′ may be prepared from compounds of formula IVd′ in a polar aprotic solvent such as DMF with an alkylating reagent such as R^{c}R^{b}-L with a base such as K₂CO₃ at temperatures from 0° C. to 150° C. such as 90° C. Compounds of formula IVd′ may be prepared from compounds of formula IVd in a mixture of a polar aprotic and protic solvent such as EtOAc and MeOH at temperatures from 0° C. to 100° C. such as 23° C. in the presence of a hydrogenation catalyst such Pd/C under a hydrogen atmosphere of from 1 to 70 psi such as 60 psi. Compounds of formula IVd may be prepared via Mitsunobu coupling with a benzyl protected compound of formula IVf with DIAD and PPh₃ in toluene at temperatures from 0° C. to 150° C. such as 50° C. Compounds of formula IVf may be prepared as described in SCHEME 7b.

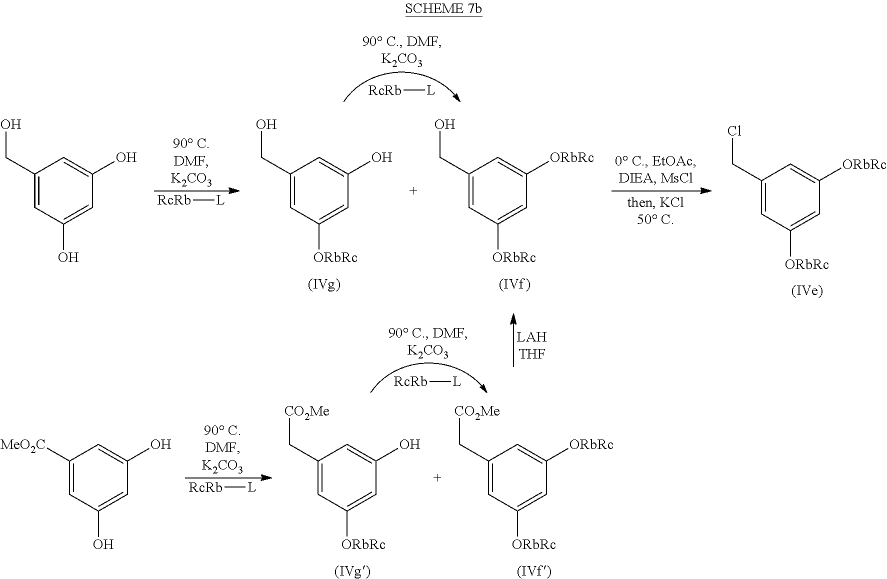

Compounds of formula IVe may be prepared from compounds of formula IVf in a polar aprotic solvent such as EtOAc in the presence of a base such as Et₃N with MsCl to yield an intermediate mesylate that is converted to the chloride in the presence of KCl with gentle heating at temperatures from 23° C. to 80° C. such as 50° C. Compounds of formula IVf may be prepared from compounds of formula IVg with a suitable alkylating reagent R^{c}R^{b}-L in a polar aprotic solvent such as DMF in the presence of a base such as K₂CO₃ at temperatures from 0° C. to 150° C. such as 90° C. Compounds of formula IVg may be prepared from an excess of 3,5-dihyroxybenzyl alcohol with a suitable alkylating reagent R^{c}R^{b}-L in a polar aprotic solvent such as DMF in the presence of a base such as potassium carbonate at temperatures from 0° C. to 150° C. such as 90° C. Compounds of formula IVf (when both R^{c}R^{b} are the same) may be prepared directly from 3,5-dihyroxybenzyl alcohol with a suitable alkylating reagent R R^{c}R^{b}-L in a polar aprotic solvent such as DMF in the presemce of a base such as K₂CO₃ at temperatures from 0° C. to 150° C. such as 90° C. Compounds of formula IVf may also be prepared from ester compounds of formula IVf′ via reduction with LAH in THF. Compounds of formula IVf′ may be prepared from compounds of formula IVg′ with a suitable alkylating reagent R^{c}R^{b}-L in a polar aprotic solvent such as DMF in the presence of a base such as K₂CO₃ at temperatures from 0° C. to 150° C. such as 90° C. Compounds of formula IVg′ may be prepared from an excess of methyl 3,5-dihyroxybenzoate with a suitable alkylating reagent R^{c}R^{b}-L in a polar aprotic solvent such as DMF in the presence of a base such as K₂CO₃ at temperatures from 0° C. to 150° C. such as 90° C. Compounds of formula IVf′ (when both R^{c}R^{b} are the same) may be prepared directly from methyl 3,5-dihyroxybenzoate with a suitable alkylating reagent R^{c}R^{b}-L in a polar aprotic solvent such as DMF in the presemce of a base such as K₂CO₃ at temperatures from 0° C. to 150° C. such as 90° C.

Certain compounds of formula IV may be prepared as shown in SCHEME 8

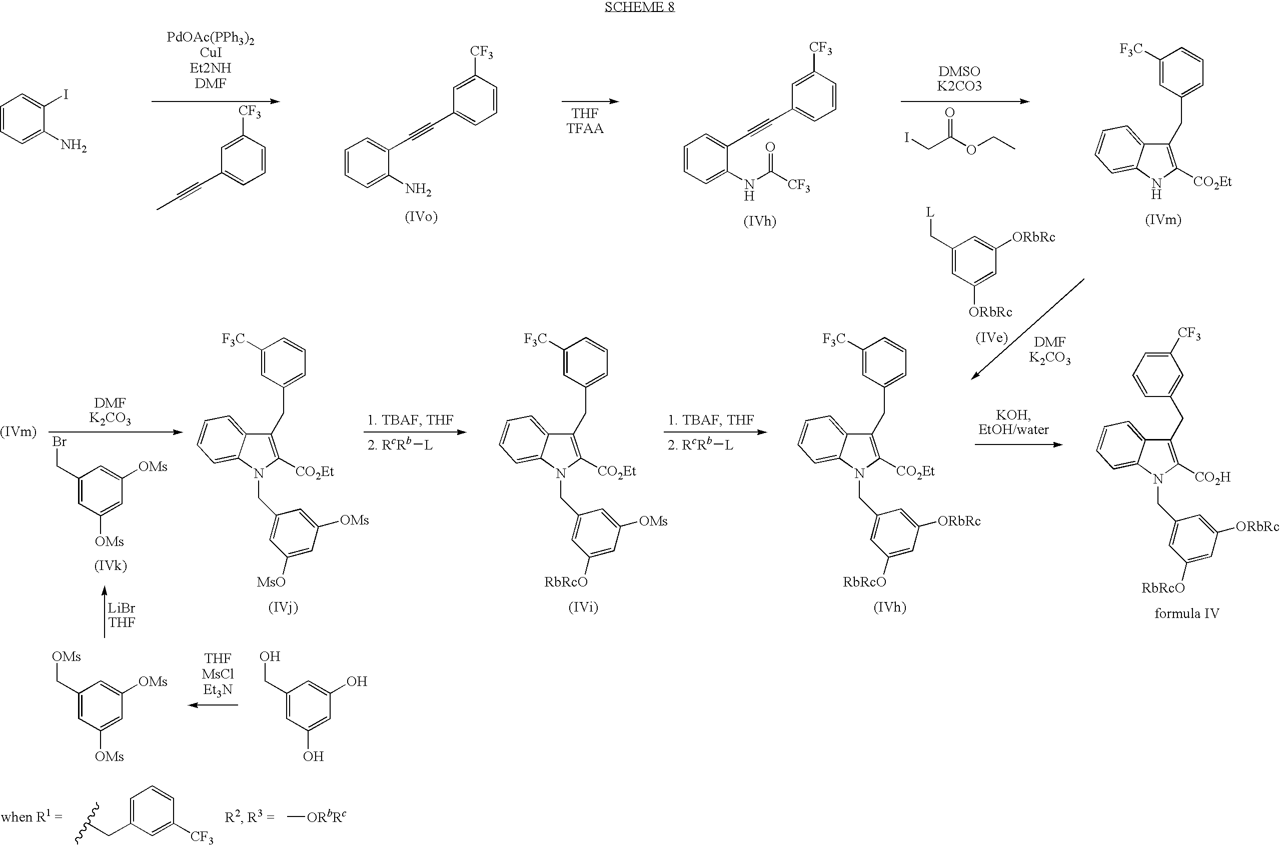

Certain compounds of formula IV may be prepared by ester hydrolysis of compounds of formula IVh in an alcohol solvent such as EtOH in the presence of water and a strong base such as KOH at temperatures from 0° C. to 100° C. such as 50° C. Compounds of formula IVh may be prepared from compounds of formula IVi by first hydrolysis of the mesylate group in a polar aprotic solvent such as THF in the presence of TBAF at temperatures from 23° C. to 120° C. such as 50° C. followed by alkylation of the resulting phenol intermediate with a suitable alkylating reagent such as R^{c}R^{b}-L in a polar aprotic solvent such as DMF in the presence of a base such as K₂CO₃ at temperatures from 23° C. to 120° C. such as 60° C. Compounds of formula IVi may be prepared from compounds of formula IVj by a similar sequence to that just described for the preparation of IVh. Compounds of formula IVj may be prepared by alkylation of compounds of formula IVm with an alkylating reagent such as a compound of formula IVk in a polar aprotic solvent such as DMF with a base such as K₂CO₃ at temperatures from 0° C. to 150° C. such as 23° C. Bromide intermediate IVk is readily available from mesylation of 3,5-dihydroxybenzyl alcohol in a polar aprotic solvent such as THF with MsCl and Et₃N followed by treatment of the per-mesylated intermediate with LiBr in a polar aprotic solvent such as THF. Compounds of formula IVm may be prepared from compounds of formula IVn in a polar aprotic solvent such as DMSO with a base such as K₂CO₃ in the presence of ethyliodoacetate at temperatures from 0° C. to 150° C. such as 80° C. Compounds of formula IVn may be prepared from compounds of formula IVo in a polar aprotic solvent such as THF in the presence of TFAA at temperatures from 0° C. to 80° C. such as 5° C. Compounds of formula IVo may be prepared from a palladium mediated coupling reaction of 2-iodoaniline with an acetylene compound such as 3-trifluoromethylphenyl acetylene in a polar aprotic solvent such as DMF in the presence of CuI and a base such as the amine base Et₂NH at temperatures from 0° C. to 100° C. such as 23° C. A suitable palladium catalyst is palladium II acetate bis-triphenylphosphine. Compounds of formula IVh may also be prepared from compounds of formula IVm via alkylation with compounds of formula IVe in a polar aprotic solvent such as DMF with a base such as K₂CO₃ at temperatures from 0° C. to 150° C. such as 23° C.

Certain compounds of formula IV may also be prepared as shown in SCHEME 9

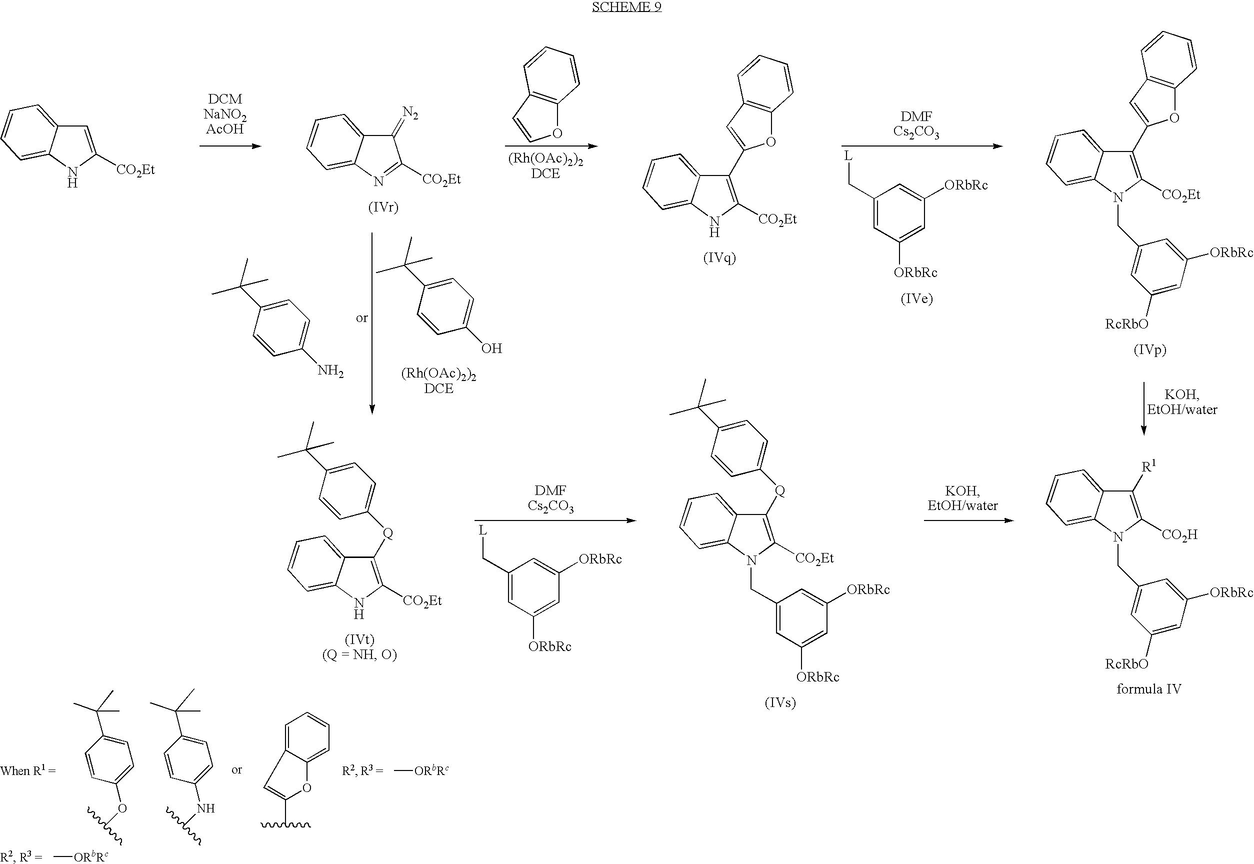

Certain compounds of formula IV may be prepared by ester hydrolysis of compounds of formula IVp in an alcohol solvent such as EtOH in the presence of water and a strong base such as KOH at temperatures from 0° C. to 100° C. such as 50° C. Compounds of formula IVp may be prepared from compounds of formula IVq via alkylation in a polar aprotic solvent such as DMF with a compound of formula IVe with a base such as Cs₂CO₃ at a temperature from 0° C. to 150° C. such as 60° C. Compounds of formula IVq may be prepared from compounds of formula IVr via a metal catalyzed coupling reaction with benzofuran and a metal such as Rh(OAc)₂)₂ in a polar solvent such as DCE at temperatures from 22° C. to 150° C. such as 80° C. Diazo compound of formula IVr may be prepared from ethyl indole-2-carboxyllic acid by methods known in the art. Compounds of formula IV may also be prepared by ester hydrolysis of compounds of formula IVs as described immediately above for IVp. Compounds of formula IVs may be prepared from compounds of formula (IVt) via alkylation in a polar aprotic solvent such as DMF with a compound of formula IVe with a base such as Cs₂CO₃ or NaHMDS in THF at temperatures from −20° C. to 100° C. such as 0° C. to 60° C. Compounds of formula IVt may be prepared from compounds of formula IVr via a (Rh(OAc)₂)₂ catalyzed coupling reaction with an alcohol (4-tertbutylphenol) or amine (4-tertbutylaniline) in a polar solvent such as DCE at temperatures from 22° C. to 150° C. such as 80° C.

Compounds of formula V may be prepared as shown in SCHEME 10

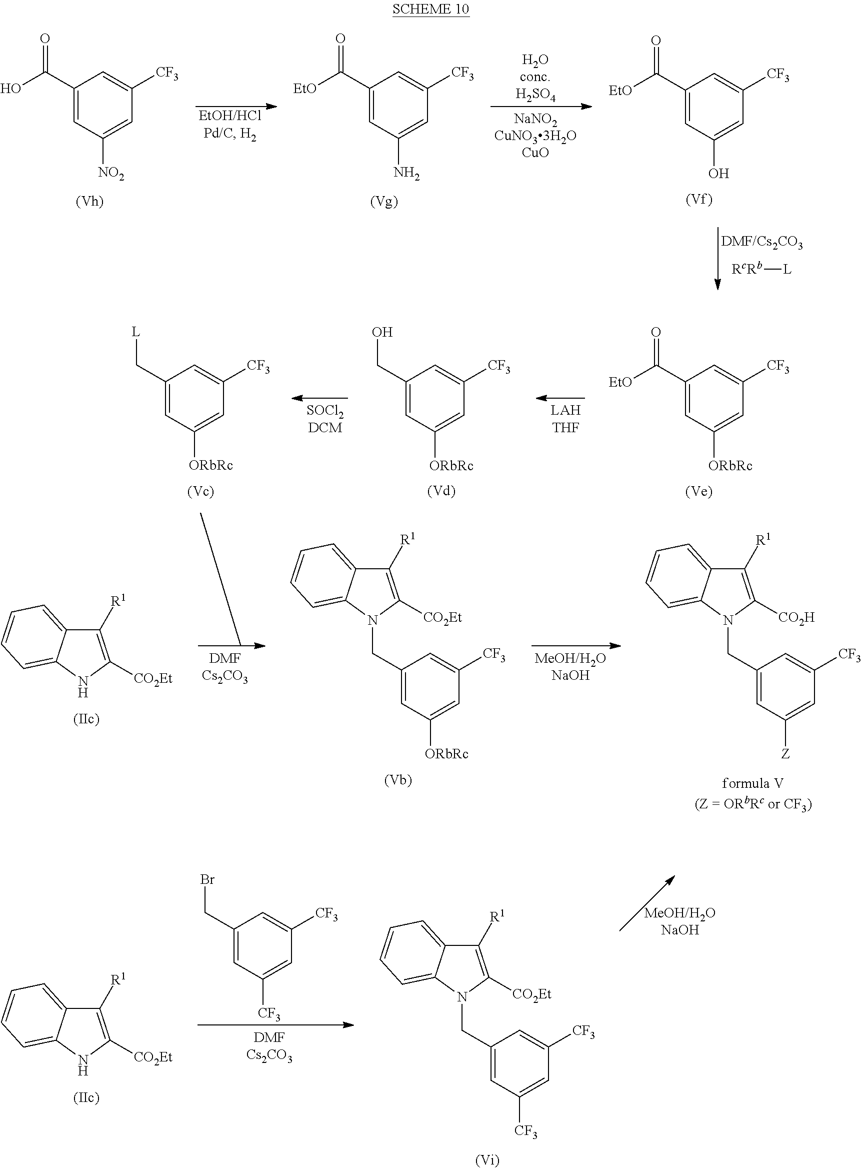

Certain compounds of formula V (Z═OR^{b}R^{c}) may be prepared from compounds of formula (Vb) in a polar protic solvent such as MeOH in the presence of water and an alkali metal hydroxide such as NaOH at temperatures from 0° C. to 150° C. such as 70° C. Compounds of formula (Vb) may be prepared from the coupling of compounds of formula IIc with compounds of formula Vc in the presence of a base such as Cs₂CO₃ (wherein L is a suitable leaving group such as a mesylate, chloride, bromide, or iodide) in a polar aprotic solvent, such as DMF, at temperatures of 0° C. to 100° C., such as 23° C. Compounds of formula Vc may be prepared from compounds of formula Vd in a polar solvent such as DCM with SOCl₂ at temperatures from 0° C. to 50° C. such as 23° C. Compounds of formula Vd may be prepared from compounds of formula Ve in a polar solvent such as THF with a reducing agent such as LAH at temperatures from 0° C. to 70° C. such as 23° C.

Compounds of formula Ve may be prepared from alkylation of phenol intermediates of formula Vf in a polar aprotic solvent such as DMF with an alkylating reagent such as R^{c}R^{b}-L and a base such as Cs₂CO₃ at temperatures from 0° C. to 100° C. such as 23° C. Phenol intermediates of formula Vf may be prepared from diazotization of aniline intermediates of formula Vg in aqueous H₂SO₄ followed by hydrolysis of the diazonium salt intermediate. A compound of formula Vg is readily prepared by one skilled in the art via hydrogenation of the nitro group from commercially available intermediate of formula Vh. Certain compounds of formula V may be prepared from compounds of formula Vi in a polar protic solvent such as MeOH in the presence of water and an alkali metal hydroxide such as NaOH at temperatures from 0° C. to 150° C. such as 70° C. Compounds of formula Vi may be prepared from the coupling of compounds with formula IIc with 3,5-trifluoromethylbenzyl bromide in the presence of a base such as Cs₂CO₃ in a polar aprotic solvent such as DMF at temperatures from 0° C. to 150° C. such as 23° C.

The following examples are set forth to illustrate the synthesis of some particular compounds of the present invention and to further exemplify particular applications of general processes described above. Accordingly, the following Example section is in no way intended to limit the scope of the invention contemplated herein.

### Intermediate Examples

### Intermediate 1a: 3-(Benzyloxy)-5-(hydroxymethyl)phenol

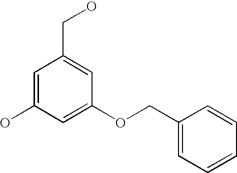

To a solution of 5.0 g (35.7 mmol) of 3,5-dihydroxybenzyl alcohol [Aldrich] in 75 mL of DMF at 0° C. was added 1.5 g (37.5 mmol) of 60% NaH. The mixture was stirred at 0° C. for 2 hrs then 4.24 mL (35.7 mmol) of benzyl bromide in 25 mL DMF was added and the solution stirred at room temperature for 12 hours. The reaction mixture was then poured into 500 mL of EtOAc, washed with three 250 mL portions of H₂O then 200 mL brine. The organic phase was dried over Na₂SO₄, filtered, concentrated, and purified by silica gel chromatography (120 grams of silica gel eluting with 0-50% EtOAc in hexanes over 45 minutes) to yield 1.41 g (17%) of 3-(benzyloxy)-5-(hydroxymethyl)phenol as a clear oil: (1H NMR 400 MHz, CDCl₃) δ 7.41-7.29 (m, 5H), 6.52 (s, 1H), 6.43 (s, 1H), 6.38 (s, 1H), 4.97 (s, 2H), 4.55 (s, 2H).

### Intermediate 1b: [3-(Benzyloxy)-5-(cyclopropylmethoxy)phenyl]methanol

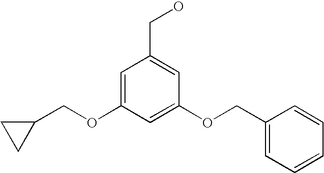

To a solution of 1.41 g (6.12 mmol) of 3-(benzyloxy)-5-(hydroxymethyl)phenol in 30 mL of DMF was added 625 uL (6.43 mmol) of cyclopropylmethylbromide and 1.70 g (12.2 mmol) of K₂CO₃ at room temperature. The mixture was stirred at room temperature for 12 hours then 200 mL of EtOAc was added. The solution was washed with three 150 mL portions of H₂O and 150 mL of brine then dried over Na₂SO₄, concentrated and purified by silica gel chromatography (120 grams of silica gel eluting with 0-50% EtOAc in hexanes over 45 minutes) to yield 1.22 g (70%) of [3-(benzyloxy)-5-(cyclopropylmethoxy)phenyl]methanol as a clear oil: 1H NMR (400 MHz CDCl₃). δ 7.43-7.28 (m, 5H), 6.60 (s, 1H), 6.53 (s, 1H), 6.48 (s, 1H), 5.04 (s, 2H), 4.62 (s, 2H), 3.78 (d, 2H, J=7.0 Hz), 1.32-1.20 (m, 1H), 0.86-0.78 (m, 2H), 0.38-0.31 (m, 2H)

### Intermediate 1c: Ethyl 1-[3-(benzyloxy)-5-(cyclopropylmethoxy)benzyl]-3-(4-tert-butylphenyl)-1H-indole-2-carboxylate

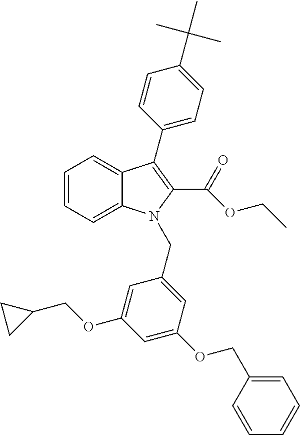

A solution of 1.04 g (3.88 mmol) of ethyl 3-bromo-1H-indole-2-carboxylate, 1.10 g (3.88 mmol) of [3-(benzyloxy)-5-(cyclopropylmethoxy)phenyl]methanol, 770 uL (3.88 mmol) of DIAD, and 1.02 g (3.88 mmol) of PPh₃ in 10 mL of toluene was stirred at room temperature for 2 hours. The solution was concentrated and the residue purified by silica gel chromatography (120 grams of silica gel eluting with 0-10% EtOAc in hexanes over 45 minutes.) The fractions containing product were concentrated and to this residue was added 630 mg (3.54 mmol) of (4-tert-butylphenyl)boronic acid, 500 mg (5.89 mmol) of NaHCO₃, and 50 mg of 10% Pd/C in 10 mL of DMF and 2 mL H₂O was stirred at 100° C. for 24 hours. The mixture was filtered through a plug of Celite and silica gel with 100 mL of EtOAc, washed with three 50 mL portions of H₂O and 100 mL of brine, dried over Na₂SO₄, then concentrated and purified by silica gel chromatography (40 grams of silica gel eluting with 0-20% EtOAc in hexanes over 45 minutes) to yield 1.20 g (86%) of ethyl 1-[3-(benzyloxy)-5-(cyclopropylmethoxy)benzyl]-3-(4-tert-butylphenyl)-1H-indole-2-carboxylate as a white foam: 1H NMR (400 MHz, CDCl₃) δ 7.62 (d, 2H, J=8.0 Hz), 7.46-7.28 (m, 11H), 7.13 (t, 1H, J=6.6 Hz), 6.39 (s, 1H), 6.34 (s, 1H), 6.29 (s, 1H), 5.72 (s, 2H), 4.93 (s, 1H), 4.10 (q, 2H, J=7.1 Hz), 3.68 (d, 2H, J=7.0 Hz), 1.38 (s, 9H), 1.22-1.18 (m, 1H), 0.96 (t, 3H, J=7.1 Hz), 0.58 (m, 2H), 0.29 (m, 2H); MS (APCI) m/z 588 (MH+).

### Intermediate 1: Ethyl 3-(4-tert-butylphenyl)-1-[3-(cyclopropylmethoxy)-5-hydroxybenzyl]-1H-indole-2-carboxylate

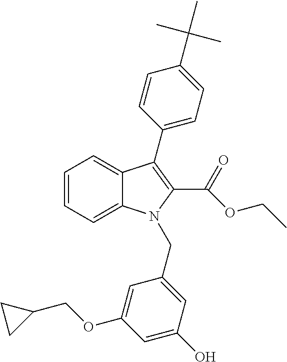

A suspension of 1.15 g (1.96 mmol) of ethyl 1-[3-(benzyloxy)-5-(cyclopropylmethoxy)benzyl]-3-(4-tert-butylphenyl)-1H-indole-2-carboxylate and 75 mg of 10% Pd/C in 2 mL MeOH and 20 mL CHCl₃ under 1 atm of H₂ was vigorously stirred for 1 hr. The solution was filtered through a plug of Celite and silica gel then concentrated to yield 950 mg (97%) of ethyl 3-(4-tert-butylphenyl)-1-[3-(cyclopropylmethoxy)-5-hydroxybenzyl]-1H-indole-2-carboxylate as a white foam: 1H NMR (400 MHz, CDCl₃). δ 7.62 (d, 1H, J=8.1 Hz), 7.45-7.29 (m, 5H), 6.30 (s, 1H), 6.24 (s, 1H), 6.08 (s, 1H), 5.69 (s, 2H), 4.81 (bs, 1H), 4.10 (q, 2H, J=6.8 Hz), 3.68 (d, 2H, J=7.0 Hz), 1.38 (s, 9H), 1.24-1.18 (m, 1H), 0.62-0.57 (m, 2H), 0.30-0.27 (m, 2H); MS (APCI) m/z 498 (MH+.)

### Intermediate 2a: 3-(Hydroxymethyl)-5-(2-methoxyethoxy)phenol

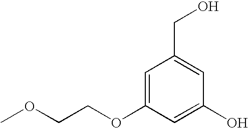

To a solution of 25.0 g (178 mmol) of 5-(hydroxymethyl)benzene-1,3-diol and 39.4 g (285 mmol) of K₂CO₃ in 150 mL DMF was added 18.4 mL (196 mmol) of 2-bromoethylmethyl ether. The solution was stirred at room temperature for 24 hours then poured in to 500 mL EtOAc. The mixture was washed with three 200 mL portions of H₂O and 200 mL brine then dried over Na₂SO₄ and concentrated. The residue was purified by silica gel chromatography (330 grams of silica gel eluting with 0-50% EtOAc in hexanes over 45 minutes) to yield 5.80 g (16%) of 3-(hydroxymethyl)-5-(2-methoxyethoxy)phenol as a clear oil: 1H NMR (400 MHz, CDCl₃) [symbol] 6.40-6.38 (m, 2H), 6.30 (s, 1H), 4.50 (s, 2H), 4.03-4.00 (m, 2H), 3.73-3.70 (m, 2H), 3.43 (s, 3H.)

### Intermediate 2b: 3-Formyl-5-(2-methoxyethoxy)phenyl pivalate

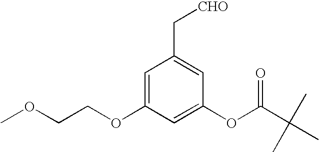

To 5.80 g (29.3 mmol) of 3-(hydroxymethyl)-5-(2-methoxyethoxy)phenol in 75 mL of DCE was added 12.7 g (146 mmol) of MnO₂. After stirring for 12 hr at room temperature, the solution was filtered through a plug of Celite and silica gel then concentrated. The residue was taken up in 100 mL CH₂Cl₂ then cooled to 0° C. and stirred while 3.80 mL (27.1 mmol) of TEA then 2.95 mL (23.8 mmol) of pivoyl chloride was added. After 12 hrs, the solution was washed with 100 mL H₂O and 100 mL brine then dried over Na₂SO₄ and concentrated to yield 6.10 g (74%) of 3-formyl-5-(2-methoxyethoxy)phenyl pivalate as a pale orange oil: 1H NMR (400 MHz, CDCl₃) δ 9.90 (s, 1H), 7.27-7.17 (m, 2H), 6.91 (s, 1H), 4.17-4.14 (m, 2H), 3.75-3.71 (m, 2H), 3.42 (s, 3H), 1.32 (s, 9H.)

### Intermediate 2c: 3-(Chloromethyl)-5-(2-methoxyethoxy)phenyl pivalate

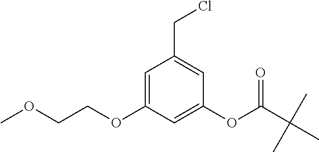

To 5.90 g (21.0 mmol) of 3-formyl-5-(2-methoxyethoxy)phenyl pivalate in 50 mL THF was added 880 mg (23.2 mmol) of NaBH₄ then stirred for 4 hrs. The reaction was quenched with 20 mL NH₄Cl (aq), 150 mL EtOAc was added then the solution was washed with two 100 mL portions of H₂O and 100 mL of brine then dried over Na₂SO₄ and concentrated. The reside was purified by silica gel chromatography (120 grams of silica gel eluting with 0-70% EtOAc in hexanes over 45 minutes.) To the purified material was added 20 mL EtOAc then cooled to 0° C. and 350 uL (2.00 mmol) DIEA, 140 uL (1.83 mmol) MsCl and 15 mg (0.17 mmol) KCl was added. After 1 hr at room temperature and 2 hrs at 50° C., the solution was washed with 50 mL H₂O and 50 mL brine then dried over Na₂SO₄ and concentrated to yield 500 mg (8%) of 3-(chloromethyl)-5-(2-methoxyethoxy)phenyl pivalate as a clear oil: 1H NMR (400 MHz, CDCl₃) δ 6.83 (s, 1H), 6.70 (s, 1H), 6.59 (s, 1H), 4.51 (s, 2H), 4.12-4.09 (m, 2H), 3.74-3.71 (m, 2H), 3.44 (s, 3H), 1.34 (s, 9H.)

### Intermediate 2: Benzyl 3-(4-tert-butylphenyl)-1-[3-hydroxy-5-(2-methoxyethoxy)benzyl]-1H-indole-2-carboxylate

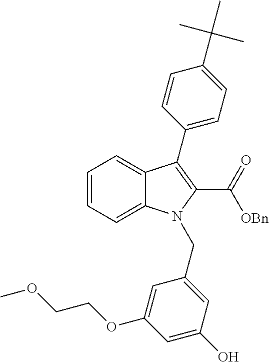

To a solution of 530 mg (1.39 mmol) of benzyl 3-(4-tert-butylphenyl)-1H-indole-2-carboxylate (intermediate 8) in 4 mL DMF was added 500 mg (1.66 mmol) of 3-(chloromethyl)-5-(2-methoxyethoxy)phenyl pivalate and 380 mg (2.77 mmol) K₂CO₃ and the mixture stirred at 60° C. for 24 hrs. The mixture was poured into 75 mL EtOAc, washed with three 50 mL portions of H₂O and 50 mL brine then dried over Na₂SO₄. Upon concentration the residue was purified by silica gel chromatography (40 grams of silica gel eluting with 0-70% EtOAc in hexanes over 45 minutes) to yield 360 mg (41%) of benzyl 3-(4-tert-butylphenyl)-1-[3-hydroxy-5-(2-methoxyethoxy)benzyl]-1H-indole-2-carboxylate as a pale yellow oil: 1h NMR (400 MHz, CDCl₃) [symbol] 7.59 (d, 1H, J=8.1 Hz), 7.41-7.10 (m, 10H), 6.91 (d, 2H, j=6.6 Hz), 6.27 (d, 2H, J=7.3 Hz), 6.06 (s, 1H), 5.69 (s, 2H), 5.12 (s, 2H), 3.99-3.96 (m, 2H), 3.67-3.64 (m, 2H), 3.42 (s, 3H), 1.37 (s, 9H.)

### Intermediate 3: Ethyl 1-(3-bromobenzyl)-3-(4-tert-butylphenyl)-1H-indole-2-carboxylate

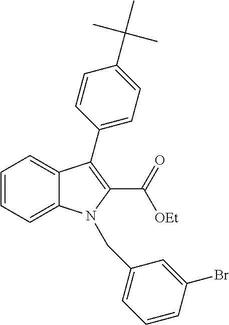

To a solution of 3.51 g (10.9 mmol) of ethyl 3-(4-tert-butylphenyl)-1H-indole-2-carboxylate in 40 mL DMF was added 3.0 g (12.0 mmol) of 3-bromobenzyl bromide and 4.52 g (32.7 mmol) of K₂CO₃ and the mixture stirred at 80° C. for 12 hrs. Another 820 mg 3-bromobenzyl bromide and 1.50 g K₂CO₃ were added and the mixture was stirred at 100° C. for 6 hrs. To the cooled mixture was added 200 mL EtOAC then washed with 150 mL 1.0 N HCl (aq), 150 H₂O and 150 mL brine then dried over Na₂SO₄. After concentration the residue was purified by silica gel chromatography (120 grams of silica gel eluting with 0-30% EtOAc in hexanes over 45 minutes) to yield 3.49 g (65%) of ethyl 1-(3-bromobenzyl)-3-(4-tert-butylphenyl)-1H-indole-2-carboxylate as a clear glass: 1H NMR (400 MHz, CDCl₃). δ 7.64 (d, 2H, J=8.2 Hz), 7.48-7.30 (m, 7H), 7.20-7.11 (m, 2H), 7.01 (d, 1H, J=7.7 Hz), 5.76 (s, 2H), 4.10 (q, 2H, J=7.2 Hz), 1.38 (s, 9H), 0.95 (t, 3H, J=7.7 Hz.)

### Intermediate 4: Ethyl 3-(4-tert-butylphenyl)-1-(3-piperazin-1-ylbenzyl)-1H-indole-2-carboxylate

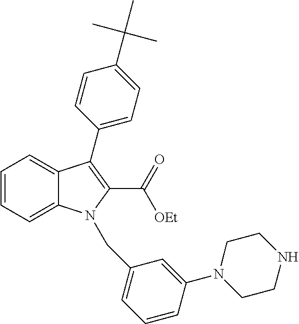

To a solution of 200 mg (0.41 mmol) of ethyl 1-(3-bromobenzyl)-3-(4-tert-butylphenyl)-1H-indole-2-carboxylate in 3.0 mL toluene was added in one portion 115 mg (0.61 mmol) tert-butyl piperazine-1-carboxylate, 98 mg (1.02 mmol) NaOtBu, 5 mg Pd(OAc)₂, and 10 uL P(t-butyl)₃ [10% in hexanes] and the mixture was stirred at room temperature for 1.5 hrs. The mixture was filtered through a plug of Celite and silica gel then 50 mL EtOAc was added and washed with 50 mL H₂O and 50 mL brine then concentrated and purified by silica gel chromatography (12 grams of silica gel eluting with 0-40% EtOAc in hexanes over 45 minutes.) The purified residue was then taken up in 5 mL CH₂Cl₂ and 1 mL TFA was added. After 1 hr at room temperature the solution was concentrated then taken up in 50 mL EtOAc and washed with 100 mL sat. Na₂CO₃ (aq) and 100 mL brine then dried over Na₂SO₄ and concentrated to yield 270 mg (54%) of ethyl 3-(4-tert-butylphenyl)-1-(3-piperazin-1-ylbenzyl)-1H-indole-2-carboxylate as a clear glass: 1H NMR (400 MHz, CDCl₃) δ 7.62 (d, 1H, J=8.2 Hz), 7.49-7.28 (m, 6H), 7.19-7.13 (m, 2H), 6.80-6.73 (m, 2H), 6.55 (d, 1H, J=7.5 Hz), 5.75 (s, 2H), 4.12 (q, 2H, J=6.9 Hz), 3.09-2.99 (m, 8H), 1.95 (bs, 1H), 1.38 (s, 9H), 0.97 (t, 3H, J=6.9 Hz); MS (ESI) m/z 495 (MH+)

### Intermediate 5: Ethyl 3-(4-tert-butylphenyl)-1-(3-thiomorpholin-4-ylbenzyl)-1H-indole-2-carboxylate

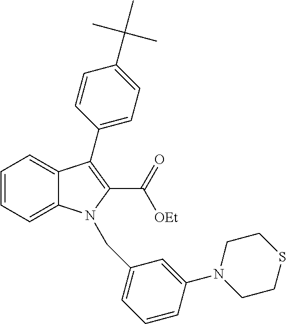

To a solution of 200 mg (0.41 mmol) ethyl 1-(3-bromobenzyl)-3-(4-tert-butylphenyl)-1H-indole-2-carboxylate in 2 mL toluene was added in one portion 49 uL (0.49 mmol) thiomorpholine, 5 mg Pd(OAc)₂, 59 mg (0.61 mmol) NaOtBu, and 10 uL triisobutylphosphatrane and the mixture stirred at 80° C. for 12 hrs. Upon cooling the mixture was filtered through a plug of Celite and silica gel with 75 mL EtOAc then washed with 50 mL 1.0 N HCl (aq), 50 ml sat. NaHCO₃ (aq) and 50 mL brine then dried over Na₂SO₄ and purified by silica gel chromatography (40 grams of silica gel eluting with 0-30% EtOAc in hexanes over 45 minutes) to yield 108 mg (52%) of ethyl 3-(4-tert-butylphenyl)-1-(3-thiomorpholin-4-ylbenzyl)-1H-indole-2-carboxylate as a white foam: 1H NMR (400 MHz, CDCl₃) δ 7.62 (d, 1H, J=7.9 Hz), 7.46-7.08 (m, 8H), 6.78-6.51 (m, 3H), 5.75 (s, 2H), 4.09 (q, 2H, J=7.0 Hz), 3.52-3.45 (m, 4H), 2.76-2.62 (m, 4H), 1.37 (s, 9H), 0.95 (t, 3H, J=7.0 Hz); MS (APCI) m/z 513 (MH+)

### Intermediate 6: 5-Bromo-2-methylbenzaldehyde

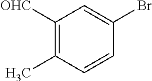

To a solution of 15.1 g (113 mmol) AlCl₃ in 30 mL CH₂Cl₂ at 0° C. was added dropwise over 20 min 7.50 mL (64.8 mmol) of 2-methylbenzaldehyde followed by the dropwise addition of 3.35 mL (64.8 mmol) Br₂ in 30 mL CH₂Cl₂ over 8 hrs at 0° C. The solution was allowed to warm to room temperature over 12 hours then was poured over 500 g ice. This mixture was extracted with 400 mL CH₂Cl₂ and the organics washed with 250 mL 1.0 N HCl (aq), 250 mL sat. NaHCO₃ (aq) and 250 mL brine then dried over Na₂SO₄. The solution was concentrated then the resulting solid was recrystallized twice from 50 mL hexanes to give 2.92 g (21%) of 5-bromo-2-methylbenzaldehyde as an off-white solid: 1H NMR (400 MHz, CDCl₃). δ 10.21 (s, 1H), 7.94 (s, 1H), 7.57 (d, 1H, J=8.5 Hz), 7.16 (d, 1H, J=8.5 Hz), 2.64 (s, 3H.)

### Intermediate 7a: 4′-(Benzyloxy)-4-methylbiphenyl-3-carbaldehyde

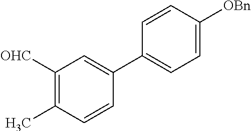

A solution of 750 mg (3.77 mmol) 5-bromo-2-methylbenzaldehyde, 1.03 g (4.52 mmol) 4-benzyloxyphenyl boronic acid, 87 mg Pd(PPh₃)₄, and 5 mL (9.42 mmol) of 2.0 M Na₂CO₃ (aq) in 15 mL DME was heated to 85° C. for 2 hrs. To the mixture was added 250 mg decolorizing carbon and the mixture stirred for 5 min then filtered through a pad of Celite and silica gel and concentrated to give 1.20 g of 4′-(benzyloxy)-4-methylbiphenyl-3-carbaldehyde as a beige solid: 1H NMR (400 MHz, CDCl₃). δ 10.36 (s, 1H), 8.02 (s, 1H), 7.69-7.62 (m, 1H), 7.55 (d, 2H, J=8.2 Hz), 7.49-7.43 (m, 1H), 7.06 (d, 2H, J=8.2 Hz), 5.13 (s, 2H), 2.71 (s, 3H.)

### Intermediate 7b: [4′-(Benzyloxy)-4-methylbiphenyl-3-yl]methanol

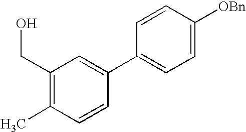

To a solution of 1.13 g (3.74 mmol) of 4′-(benzyloxy)-4-methylbiphenyl-3-carbaldehyde in 15 mL THF was added 142 mg (3.74 mmol) NaBH₄ and the mixture stirred at room temperature for 12 hrs. To the mixture was then added 75 mL EtOAc and then washed with 100 mL H₂O and 100 mL brine then dried over Na₂SO₄ and concentrated. The resulting solid was recrystallized from EtOAc and hexanes to give 720 mg (63%) of [4′-(benzyloxy)-4-methylbiphenyl-3-yl]methanol as a white solid: 1H NMR (400 MHz, CDCl₃). δ 7.58-7.51 (m, 3H), 7.48-7.34 (m, 6H), 7.22 (d, 1H, J=7.8 Hz), 7.06 (d, 2H, J=8.1 Hz), 5.13 (s, 2H), 4.77 (s, 2H), 2.39 (s, 3H.)

### Intermediate 7c: Ethyl 1-{[4′-(benzyloxy)-4-methylbiphenyl-3-yl]methyl}-3-(4-tert-butylphenyl)-1H-indole-2-carboxylate

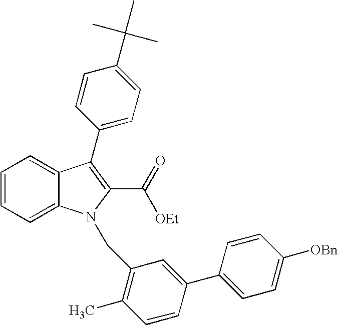

To 300 mg (0.99 mmol) [4′-(benzyloxy)-4-methylbiphenyl-3-yl]methanol in 5 mL CH₂Cl₂ was added 92 uL (1.18 mmol) MsCl and 275 uL TEA and the solution stirred at room temperature for 12 hrs. The solution was washed with 15 mL H₂O and 15 mL brine then dried over Na₂SO₄ and concentrated. To this residue was added 7 mL of CH₃CN followed by 410 mg (2.96 mmol) and 300 mg (0.99 mmol) of ethyl 3-(4-tert-butylphenyl)-1H-indole-2-carboxylate then the mixture was stirred at 80° C. for 12 hrs. To the cooled solution was added 75 mL EtOAc. The mixture was then washed with 50 mL H₂O and 50 mL brine and dried over Na₂SO₄ and concentrated to yield 590 mg (99%) of ethyl 1-{[4′-(benzyloxy)-4-methylbiphenyl-3-yl]methyl}-3-(4-tert-butylphenyl)-1H-indole-2-carboxylate as an off-white solid: 1H NMR (400 MHz, CDCl₃). δ 7.64 (d, 1H, J=8.6 Hz), 7.49-7.36 (m, 8H), 7.36-7.12 (m, 4H), 6.89 (d, 2H, J=8.2 Hz), 6.58 (s, 1H), 5.05 (s, 2H), 4.08 (q, 2H, J=7.8 Hz), 2.44 (s, 3H), 1.38 (s, 9H), 0.97 (t, 3H, J=7.8 Hz.)

### Intermediate 7: Ethyl 3-(4-tert-butylphenyl)-1-[(4′-hydroxy-4-methylbiphenyl-3-yl)methyl]-1H-indole-2-carboxylate

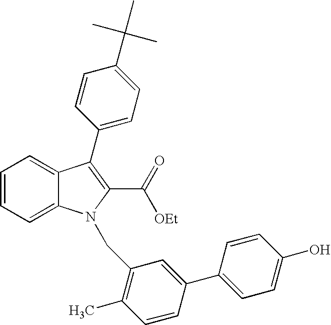

A solution of 700 mg (1.18 mmol) of ethyl 1-{[4′-(benzyloxy)-4-methylbiphenyl-3-yl]methyl}-3-(4-tert-butylphenyl)-1H-indole-2-carboxylate and 50 mg 10% Pd/C in 10 ml CHCl₃ and 1 mL MeOH was stirred vigorously under 1 atm H₂ for 12 hrs. The solution was filtered through a plug of Celite and silica gel then concentrated and purified by silica gel chromatography (40 grams of silica gel eluting with 0-40% EtOAc in hexanes over 45 minutes) to yield 410 mg (67%) of ethyl 3-(4-tert-butylphenyl)-1-[(4′-hydroxy-4-methylbiphenyl-3-yl)methyl]-1H-indole-2-carboxylate as a white foam: 1H NMR (400 MHz, CDCl₃) δ 7.67 (s, 1H, J=8.1 Hz), 7.48-7.43 (m, 4H), 7.31-7.22 (m, 4H), 7.17-7.14 (m, 3H), 6.74 (d, 2H, J=8.6 Hz), 6.57 (s, 1H), 4.89 (bs, 1H), 4.07 (q, 2H, J=7.2 Hz), 2.46 (s, 3H), 1.39 (s, 9H), 0.91 (t, 3H, J=7.2 Hz.)

### Intermediate 8: Benzyl 3-(4-tert-butylphenyl)-1H-indole-2-carboxylate

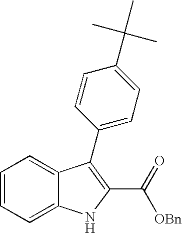

To 25.0 g (77.8 mmol) of ethyl 3-(4-tert-butylphenyl)-1H-indole-2-carboxylate in 350 mL EtOH was added 13.1 g (233 mmol) KOH in 50 mL H₂O and the solution refluxed for 2 hrs. The solution was concentrated to ⅓ volume then slowly made acidic to litmus with 2.0 N HCl (aq) and extracted with two 300 mL portions of EtOAc. The combined organics were washed with 250 mL H₂O and 200 mL brine then dried over Na₂SO₄ and concentrated. The residue was taken up in 300 mL DMF then 21.7 mL (156 mmol) TEA and 9.70 mL (81.7 mmol) benzyl bromide were added and the mixture stirred at room temperature for 4 hrs. Another 4.60 mL (39 mmol) of benzyl bromide was added and the mixture stirred for 12 hrs. To the mixture was added 750 mL EtOAc then the solution was washed with 500 mL 1.0 N HCl (aq), two 250 mL portions of 1.0 N NaOH (aq) and 250 mL of brine then dried over Na₂SO₄ and concentrated to yield 19.67 g (66%) of benzyl 3-(4-tert-butylphenyl)-1H-indole-2-carboxylate as a pale yellow solid: 1H NMR (400 MHz, CDCl₃) δ 9.11 (bs, 1H), 7.68 (d, 1H, J=8.2 Hz), 7.49 (d, 2H, J=8.3 Hz), 7.44-7.31 (m, 7H), 7.25-7.22 (m, 2H), 7.16-7.13 (m, 1H), 5.31 (s, 2H), 1.40 (s, 9H.)

### Intermediate 9a: 4-Bromo-2-(chloromethyl)-1-methylbenzene

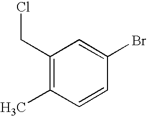

To a solution of 2.50 g (12.6 mmol) 5-bromo-2-methylbenzaldehyde in 40 mL THF was added 570 mg (15.1 mmol) of NaBH₄ and the mixture stirred at room temperature for 1 hr. The reaction was quenched with sat. NHCl₄ (aq) then extracted with 150 mL EtOAc. The organics were washed with two 50 mL portions of H₂O and 50 mL brine then dried over Na₂SO₄ and concentrated. The residue was taken up in 75 mL EtOAc, cooled to 0° C. and 5 drops of pyridine then 960 uL (13.2 mmol) of SOCl₂ was added and stirred at room temperature for 12 hrs. The solution was washed with 50 mL 1.0 N HCl (aq), 50 mL sat. NaHCO₃ (aq) and 50 mL brine then dried over Na₂SO₄ and concentrated to give 2.30 g (84%) of 4-bromo-2-(chloromethyl)-1-methylbenzene as a pale yellow oil: 1H NMR (400 MHz, CDCl₃). δ 7.46 (s, 1H), 7.35 (d, 1H, J=8.1 Hz), 7.06 (d, 1H, J=8.1 Hz), 4.53 (s, 2H), 2.36 (s, 3H.)

### Intermediate 9b: Benzyl 1-(5-bromo-2-methylbenzyl)-3-(4-tert-butylphenyl)-1H-indole-2-carboxylate

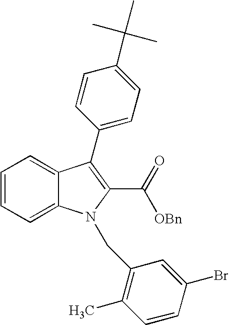

A solution of 1.0 g (2.61 mmol) of benzyl 3-(4-tert-butylphenyl)-1H-indole-2-carboxylate, 715 mg (3.26 mmol) 4-bromo-2-(chloromethyl)-1-methylbenzene, and 1.08 g (7.82 mmol) K₂CO₃ in 8 mL DMF was stirred at 100° C. for 12 hrs. To the cooled mixture was added 75 mL EtOAc and the mixture was washed with three 50 mL portions of H₂O and 50 mL brine then dried over Na2SO4 and purified by silica gel chromatography (40 grams of silica gel eluting with 0-30% EtOAc in hexanes over 45 minutes) to yield 1.06 g (68%) of benzyl 1-(5-bromo-2-methylbenzyl)-3-(4-tert-butylphenyl)-1H-indole-2-carboxylate as a white foam: 1H NMR (400 MHz, CDCl₃) δ 7.64 (d, 1H, J=8.1 Hz), 7.43-7.17 (m, 11H), 7.05 (d, 1H, J=6.9 Hz), 6.90 (s, 1H), 5.67 (s, 2H), 5.09 (s, 2H), 2.33 (s, 3H), 1.38 (s, 9H)

### Intermediate 9: 3′-{[2-[(Benzyloxy)carbonyl]-3-(4-tert-butylphenyl)-1H-indol-1-yl]methyl}-4′-methylbiphenyl-4-carboxylic acid

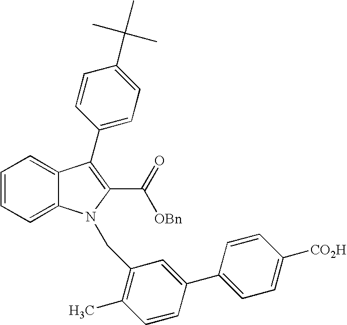

To a solution of 500 mg (0.88 mmol) of benzyl 1-(5-bromo-2-methylbenzyl)-3-(4-tert-butylphenyl)-1H-indole-2-carboxylate in 5 mL DMF and 0.5 mL H₂O was added in one portion 220 mg (1.32 mmol) 4-carboxylphenyl boronic acid, 50 mg 10% Pd/C, and 220 mg (2.65 mmol) NaHCO₃ and the mixture stirred at 90° C. for 12 hrs. The cooled mixture was filtered through a plug of Celite and silica gel then the plug was washed with 15 mL of a 5:1 mixture of DMF and H₂O. To the combined filtrate was slowly slowly added with vigorous stirring 40 mL 1.0 N HCl (aq). The resulting solids were collected by suction filtration, washed with H₂O and dried to give 460 mg (86%) of 3′-{[2-[(benzyloxy)carbonyl]-3-(4-tert-butylphenyl)-1H-indol-1-yl]methyl}-4′-methylbiphenyl-4-carboxylic acid as a white solid: 1H NMR (400 MHz, DMSO-d6). δ 12.90 (bs, 1H), 8.05 (d, 1H, J=8.0 Hz), 7.88-7.80 (m, 3H), 7.59-7.51 (m, 3H), 7.49-7.26 (m, 8H), 7.21-7.11 (m, 3H), 6.84 (d, 2H, J=8.1 Hz), 6.39 (s, 1H), 5.82 (s, 2H), 5.07 (s, 2H), 2.41 (s, 3H), 1.36 (s, 9H); MS (ESI) m/z 608 (MH+)

### Intermediate 10: Ethyl 1-(5-bromo-2-methylbenzyl)-3-(4-tert-butylphenyl)-1H-indole-2-carboxylate

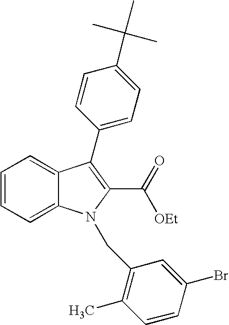

A solution of 2.53 g (11.5 mmol) of 4-bromo-2-(chloromethyl)-1-methylbenzene, 3.09 g (9.60 mmol) ethyl 3-(4-tert-butylphenyl)-1H-indole-2-carboxylate, and 3.98 g (28.8 mmol) K₂CO₃ in 40 mL DMF was stirred at 90° C. for 12 hrs. To the cooled solution was added 200 mL EtOAc then the mixture was washed with 100 mL H₂O and 100 mL brine then dried over Na₂SO₄ and concentrated. The residue was then recrystallized from EtOAc and hexanes to give 3.46 g (71%) of ethyl 1-(5-bromo-2-methylbenzyl)-3-(4-tert-butylphenyl)-1H-indole-2-carboxylate as a white solid: 1H NMR (400 MHz, CDCl₃). δ 7.67 (d, 1H, J=8.0 Hz), 7.48-7.43 (m, 4H), 7.33-7.15 (m, 4H), 7.07 (d, 1H, J=8.1 Hz), 6.59 (s, 1H), 5.71 (s, 2H), 4.08 (q, 2H, J=7.1 Hz), 2.40 (s, 3H), 1.40 (s, 9H), 0.93 (t, 3H, J=7.1 Hz)

### Intermediate 11a: 3-(Benzyloxy)-5-hydroxybenzaldehyde

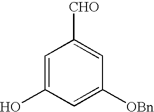

To a solution of 2.33 g (10. mmol) of 3-(benzyloxy)-5-(hydroxymethyl)phenol in 25 DCE was added 4.40 g (50.6 mmol) of MnO₂ then stirred at room temperature for 12 hrs. The mixture was then filtered through a pad of Celite and silica gel then concentrated to give 1.57 g (68%) of 3-(benzyloxy)-5-hydroxybenzaldehyde as a tan solid: 1H NMR (400 MHz, CDCl₃) δ 9.87 (s, 1H), 7.43-7.33 (m, 5H), 7.08 (s, 1H), 6.96 (s, 1H), 6.75 (s, 1H), 5.28 (bs, 1H), 5.09 (s, 2H)

### Intermediate 11b: 3-(Benzyloxy)-5-formylphenyl trifluoromethanesulfonate

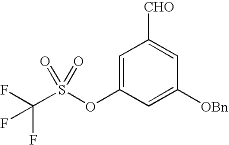

To a stirred solution of 1.56 g (6.83 mmol) of 3-(benzyloxy)-5-hydroxybenzaldehyde and 2.85 mL (20.5 mmol) TEA in 20 mL CH₂Cl₂ at 0° C. was added 2.90 mL (17.1 mmol) Tf₂O. The solution was stirred at room temperature for 30 min then washed with 25 mL sat. NaHCO₃ (aq), 25 mL H₂O, and 25 mL brine then dried over Na₂SO₄ and concentrated to yield 2.19 g (89%) of 3-(benzyloxy)-5-formylphenyl trifluoromethanesulfonate as a brown oil: 1H NMR (400 MHz, CDCl₃). δ 9.97 (s, 1H), 7.56 (s, 1H), 7.52-7.38 (m, 6H), 7.17 (s, 1H), 5.16 (s, 2H)

### Intermediate 11c: Methyl 3′-(benzyloxy)-5′-formylbiphenyl-4-carboxylate

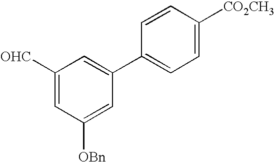

In one portion was added 1.40 g (8.41 mmol) of 4-carboxyphenylboronic acid, 150 mg Pd(PPh₃)₄ and 8.40 mL (16.8 mmol) 2.0 M Na₂CO₃ (aq) to a stirred solution of 2.02 g (5.61 mmol) of 3-(benzyloxy)-5-formylphenyl trifluoromethanesulfonate in 25 mL DME. The mixture was stirred vigorously for 5 hrs after which the cooled solution was filtered through a plug of Celite and silica gel with 100 mL EtOAc. The filtrate was washed with 100 mL H₂O and 100 mL brine then dried over Na₂SO₄ and concentrated. The residue was taken up in 20 mL DMF then 1.23 mL (19.7 mmol) CH₃I and 2.72 g (19.7 mmol) K₂CO₃ were added and the mixture stirred at room temperature for 1 hr. To this mixture was added 150 mL EtOAc then was washed with three 100 mL portions of H₂O and 100 mL of brine then dried over Na₂SO₄, concentrated and purified by silica gel chromatography (120 grams of silica gel eluting with 0-30% EtOAc in hexanes over 45 minutes) to yield 940 mg (48%) of methyl 3′-(benzyloxy)-5′-formylbiphenyl-4-carboxylate as a beige solid: 1H NMR (400 MHz, CDCl₃). δ 10.04 (s, 1H), 8.13 (d, 1H, J=8.7 Hz), 7.73 (s, 1H), 7.67 (d, 2H, J=8.5 Hz), 7.55-7.38 (m, 7H), 5.19 (s, 2H), 3.95 (s, 3H)

### Intermediate 11d: Methyl 3′-(benzyloxy)-5′-(chloromethyl)biphenyl-4-carboxylate

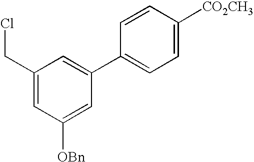

To a stirred solution of 940 mg (2.71 mmol) of 3′-(benzyloxy)-5′-formylbiphenyl-4-carboxylate in 10 mL THF was added 125 mg (3.26 mmol) NaBH₄ and the mixture stirred at room temperature for 1 hr. The reaction was quenched with sat NHCl₄ (aq) then extracted with two 50 mL portions of EtOAc. The combined organics were washed with 100 mL H₂O and 100 mL brine then dried over Na₂SO₄ and concentrated. The residue was taken up in 10 mL EtOAc, cooled to 0° C., then 210 uL (2.85 mmol) SOCl₂ and 2 drops of pyridine were added and the solution stirred at room temperature for 12 hrs. The solution was then washed with 20 mL 0.5 N HCl (aq), 20 mL sat. NaHCO₃ (aq) and 20 mL brine then dried over Na₂SO₄ and concentrated to yield 940 mg (94%) of methyl 3′-(benzyloxy)-5′-(chloromethyl)biphenyl-4-carboxylate as a white solid: 1H NMR (400 MHz, CDCl₃) δ 8.09 (d, 2H J=8.4 Hz), 7.62 (d, 2H, J=8.2 H), 7.49-7.32 (m, 5H), 7.24 (d, 1H, J=7.3 Hz), 7.18 (s, 1H), 7.05 (s, 1H), 5.13 (s, 2H), 4.61 (s, 2H), 3.94 (s, 3H)

### Intermediate 11: Ethyl 3-(4-tert-butylphenyl)-1-{[5-hydroxy-4′-(methoxycarbonyl)biphenyl-3-yl]methyl}-1H-indole-2-carboxylate

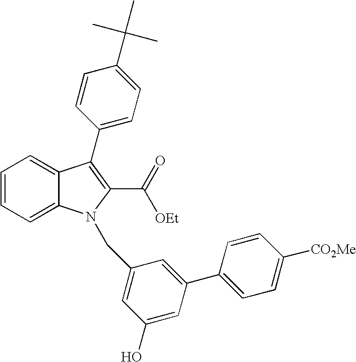

To a stirred solution of 940 mg (2.56 mmol) of methyl 3′-(benzyloxy)-5′-(chloromethyl)biphenyl-4-carboxylate in 8 mL DMF was added 690 mg (2.14 mmol) of ethyl 3-(4-tert-butylphenyl)-1H-indole-2-carboxylate and 740 mg (5.34 mmol) K₂CO₃ and the mixture stirred at 80° C. for 5 hrs. To the cooled mixture was added 75 mL EtOAc and the solution washed with three 75 mL portions of H₂O, 75 mL of brine then dried over Na₂SO₄ and concentrated. To this residue was added 30 mL CHCl₃, 5 mL MeOH and 200 mg 10% Pd/C then the mixture was shaken under 20 psi H₂ for 20 min. The reaction mixture was then filtered through a plug of Celite and silica gel, concentrated then purified by silica gel chromatography (120 grams of silica gel eluting with 0-50% EtOAc in hexanes over 45 minutes) to yield 950 mg (79%) of ethyl 3-(4-tert-butylphenyl)-1-{[5-hydroxy-4′-(methoxycarbonyl)biphenyl-3-yl]methyl}-1H-indole-2-carboxylate as a white foam: 1H NMR (400 MHz, CDCl₃). δ 8.05 (d, 2H, J=8.2 Hz), 7.64 (d, 1H, J=8.1 Hz), 7.55 (d, 2H, 8.2 Hz), 7.49-7.38 (m, 6H), 7.14 (t, 1H, 7.3 Hz), 7.04 (s, 1H), 6.94 (s, 1H), 6.48 (s, 1H), 5.81 (s, 2H), 5.07 (bs, 1H), 4.09 (q, 2H, J=7.1 Hz), 3.92 (s, 3H), 1.39 (s, 9H), 0.94 (t, 3H, J=7.1 Hz)

### Intermediate 12: 3′-[(3-[4-(1,1-Dimethylethyl)phenyl]-2-{[(phenylmethyl)oxy]carbonyl}-1H-indol-1-yl)methyl]-4′-methyl-3-biphenylcarboxylic acid

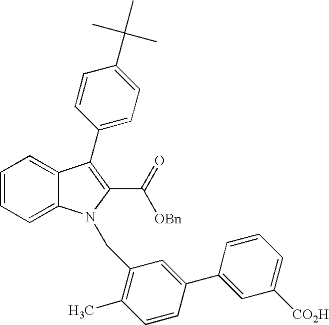

The title compound was obtained in 92% yield from 3-(dihydroxyboranyl)benzoic acid and benzyl 1-(5-bromo-2-methylbenzyl)-3-(4-tert-butylphenyl)-1H-indole-2-carboxylate as described for the synthesis of Intermediate 9: 1H NMR (400 MHz, DMSO-d6). δ 13.07 (bs, 1H), 8.18 (s, 1H), 7.96 (d, 2H, J=7.8 Hz), 7.79-7.77 (m, 2H), 7.62-7.24 (m, 6H), 7.21-7.10 (m, 2H), 6.86 (d, 2H, J=7.5 Hz), 5.81 (s, 2H), 5.07 (s, 2H), 2.38 (s, 3H), 1.32 (s, 9H)

### Intermediate 13: 1,1-Dimethylethyl 1-[(5-bromo-2-methylphenyl)methyl]-3-[4-(1,1-dimethylethyl)phenyl]-1H-indole-2-carboxylate

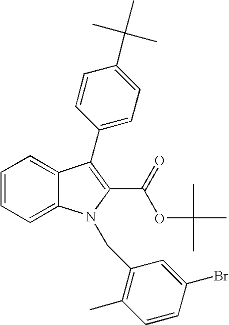

To 10.0 g (31.1 mmol) of ethyl 3-[4-(1,1-dimethylethyl)phenyl]-1H-indole-2-carboxylate in 40 mL THF, 40 mL EtOH and 20 mL H₂O was added 5.0 g (0.124 mol) NaOH and the solution stirred at 80° C. for 2 hr. The solution was concentrated to dryness and the residue taken up in 500 mL H₂O and 250 mL EtOAc. The aqueous layer was separated, washed with 150 mL EtOAc then the pH was lowered to 5.0 with 1.0 N HCl (aq). The solution was extracted with two 200 mL portions of EtOAc. The combine organics were washed with 250 mL brine then dried over Na₂SO₄ and concentrated. To the residue was added 30 mL toluene followed by 7.40 mL (30.7 mmol) of {bis[(1,1-dimethylethyl)oxy]methyl}dimethylamine and the solution stirred at 90° C. for 6 hr. 200 mL of EtOAc was added then the mixture was washed with three 150 mL portions of H₂O and 150 mL brine then dried over Na₂SO₄ and concentrated to dryness. The residue was recrystallized from EtOAc/hexanes to give colorless crystals. To 700 mg (2.00 mmol) of this solid was added 830 mg (6.01 mmol) K₂CO₃, 530 mg (2.41 mmol) 4-bromo-2-(chloromethyl)-1-methylbenzene and 10 mL DMF and the mixture stirred at 100° C. for 8 hr. To this solution was added 75 mL EtOAc and the solution washed with three 50 mL portions of H₂O and 50 mL brine then dried over Na₂SO₄ and concentrated to give 1.07 g (95% overall yield) of 1,1-dimethylethyl 1-[(5-bromo-2-methylphenyl)methyl]-3-[4-(1,1-dimethylethyl)phenyl]-1H-indole-2-carboxylate as a tan solid: 1H NMR (400 MHz, CDCl₃) δ 7.67 (d, 1H, J=7.8 Hz), 7.47 (d, 2H, J=7.8 Hz), 7.47 (d, 2H, J=7.8 Hz), 7.36-7.28 (m, 2H), 7.21-7.15 (m, 2H), 7.06 (d, 1H, J=7.8 Hz), 6.68 (s, 1H), 5.68(s, 2H), 2.39 (s, 3H), 1.41 (s, 9H), 1.22 (s, 9H); MS (ESI) m/z 478 (M-tert-butyl, 100%) 534 (MH+, 10%)

### Intermediate 14: 3′-({2-{[(1,1-Dimethylethyl)oxy]carbonyl}-3-[4-(1,1-dimethylethyl)phenyl]-1H-indol-1-yl}methyl)-4′-methyl-3-biphenylcarboxylic acid

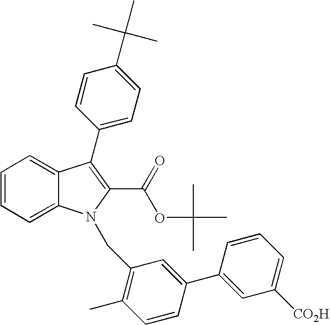

To a solution of 315 mg (0.59 mmol) of 1,1-dimethylethyl 1-[(5-bromo-2-methylphenyl)methyl]-3-[4-(1,1-dimethylethyl)phenyl]-1H-indole-2-carboxylate (Intermediate 13), 150 mg (1.77 mmol) of NaHCO₃, and 150 mg (0.90 mmol) 3-(dihydroxyboranyl)benzoic acid in 4 mL DMF and 1 mL H₂O was added 50 mg Pd/C (10%, Degussa type) and the mixture stirred at 90° C. for 12 hr. An additional 75 mg (0.45 mmol) 3-(dihydroxyboranyl)benzoic acid and 75 mg (0.88 mmol) NaHCO₃ added and the mixture stirred for an additional 24 hr. The solution was filtered through a plug of Celite and the pad washed with 5 mL DMF. The combined organics were poured into 25 mL 1.0 N HCl (aq) and the resulting solids collected by suction filtration, washed with H₂O and dried to yield 330 mg (99%) of 3-({2-{[(1,1-dimethylethyl)oxy]carbonyl}-3-[4-(1,1-dimethylethyl)phenyl]-1H-indol-1-yl}methyl)-4′-methyl-3-biphenylcarboxylic acid as a white solid: 1H NMR (400 MHz, DMSO-d6) δ 13.05 (bs, 1H), 8.18 (s, 1H), 7.99-7.96 (m, 2H), 7.82-7.71 (m, 1H), 7.62-7.41 (m, 6H), 7.39-7.22 (m, 3H), 7.17-7.14 (m, 1H), 6.41 (s, 1H), 5.79 (s, 2H), 1.38 (s, 9H), 1.09 (s, 9H); MS (ESI) m/z 596 (M+Na)

### Intermediate 15: 3′-({2-{[(1,1-dimethylethyl)oxy]carbonyl}-3-[4-(1,1-dimethylethyl)phenyl]-1H-indol-1-yl}methyl)-4′-methyl-4-biphenylcarboxylic acid

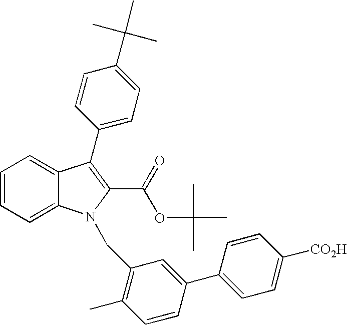

Intermediate 15 was obtained in 77% yield as a white solid from 1,1-dimethylethyl 1-[(5-bromo-2-methylphenyl)methyl]-3-[4-(1,1-dimethylethyl)phenyl]-1H-indole-2-carboxylate (intermediate 13) using 4-(dihydroxyboranyl)benzoic acid as described for the synthesis of Intermediate 14: 1H NMR (400 MHz, DMSO-d6). δ 12.97 (bs, 1H), 7.84-7.80 (m, 2H), 7.63-7.42 (m, 5H), 7.42-7.25 (m, 5H), 7.18-7.07 (m, 2H), 6.39 (s, 1H), 5.78 (s, 2H), 1.37 (s, 9H), 1.05 (s, 9H); MS (ESI) m/z 518 (M-tert-butyl, 100%)

### Intermediate 16: phenylmethyl 3-[4-(1,1-dimethylethyl)phenyl]-1-{[3-(1-piperazinyl)phenyl]methyl}-1H-indole-2-carboxylate

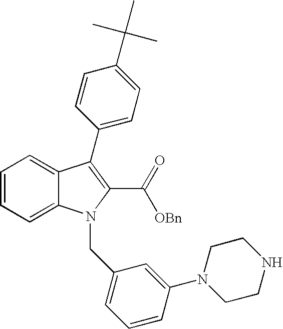

To a solution of 750 mg (1.96 mmol) of benzyl 3-(4-tert-butylphenyl)-1H-indole-2-carboxylate (Intermediate 8) and 730 mg (2.93 mmol) of 1-bromo-3-(bromomethyl)benzene in 6 mL DMF was added 810 mg (5.87 mmol) K₂CO₃ and the mixture stirred at 100° C. for 12 hr. 50 mL EtOAc was added and the mixture washed with three 25 mL portions of H₂O and 25 mL brine, dried over Na₂SO₄, concentrated and purified by silica gel chromatography (40 grams of silica gel eluting with 0-20% EtOAc in hexanes over 45 minutes.) The fractions containing product were combined and concentrated. To this residue was added 365 mg (1.95 mmol) 1,1-dimethylethyl 1-piperazinecarboxylate, 10 mg Pd(OAc)₂, 20 uL tri-tertbutylphosphine (10% in hexanes), 315 mg (3.26 mmol) of NaOtBu and 10 ml toluene. The mixture was stirred at room temperature for 12 hr. The solution was filtered through a pad of Celite and the pad washed with 50 mL EtOAc. The combine organics were washed with 50 mL H₂O and 50 mL brine, dried over Na₂SO₄, concentrated and purified by silica gel chromatography (40 grams of silica gel eluting with 0-25% EtOAc in hexanes over 45 minutes.) The fractions containing product were combined and concentrated and the residue was taken up in 5 mL CH₂Cl₂ and 1 mL TFA. After stirring at room temperature for 1 hr the solution was concentrated to dryness. The residue was taken up in 50 mL EtOAc, washed with 25 mL sat. Na₂CO₃ (aq) and 25 mL brine then dried over Na₂SO₄ and concentrated to yield 260 mg (24%) of the title compound as a pale yellow glass: 1H NMR (400 MHz, CDCl₃) δ 7.60 (d, 1H, J=8.2 Hz), 7.61-7.48 (m 7H), 7.46-7.12 (m, 4H), 6.90 (d, 2H, J=6.6 Hz), 6.76 (d, 1H, J=8.1 Hz), 6.69 (s, 1H), 6.53 (d, 1H, J=7.4 Hz), 5.6 (s, 2H), 5.11 (s, 2H), 3.09-2.92 (m, 8H), 1.38 (s, 9H); MS (ESI) m/z 558 (MH+)

### Intermediate 17a: Methyl 3′-formylbiphenyl-4-carboxylate

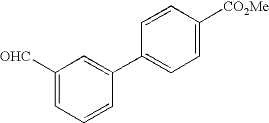

A solution of 0.25 mL (2.14 mmol) of 3-bromobenzaldehyde, 710 mg (4.29 mmol) of 4-carboxyphenylboronic acid, 50 mg of palladium tetrakis and 3.5 mL (6.42 mmol) of 2.0 M Na₂CO₃ (aq) in 10 mL CH₃CN was stirred at 90° C. for 12 hrs. The cooled solution was filtered through a plug of Celite and silica gel with 75 EtOAc then the organics were washed with 50 mL H₂O and 50 mL brine then dried over Na₂SO₄ and concentrated. To the residue was added 380 uL (6.17 mmol) CH₃I, 1.14 g (8.22 mmol) and 15 mL DMF and the mixture stirred at room temperature for 2 hrs. To the mixture was added 100 mL EtOAc then the organics were washed with three 75 mL portions of H₂O, 75 mL brine then dried over Na₂SO₄ and then purified by silica gel chromatography (40 grams of silica gel eluting with 0-10% EtOAc in hexanes over 45 minutes) to give 420 mg (82%) of methyl 3′-formylbiphenyl-4-carboxylate as a white solid: 1H NMR (400 MHz, CDCl₃). δ 10.11 (s, 1H), 8.16-8.13 (m, 3H), 7.92-7.89 (m, 2H), 7.71 (d, 2H, J=7.3 Hz), 7.70 (t, 1H, J=7.3 Hz), 3.96 (s, 3H)

### Intermediate 17b: Ethyl 3-bromo-1-{[4′-(methoxycarbonyl)biphenyl-3-yl]methyl}-1H-indole-2-carboxylate

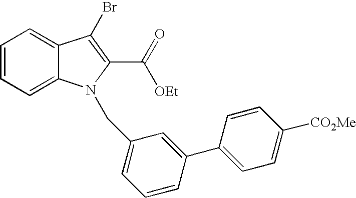

To 820 mg (3.41 mmol) of methyl 3′-formylbiphenyl-4-carboxylate in 20 mL EtOH was added 130 mg (3.41 mmol) of NaBH₄ then the mixture was stirred at room temperature for 2 hrs. The reaction was quenched with sat. NHCl₄ (aq) and 100 mL Et₂O was added. The organics were washed with 75 mL H₂O and 75 mL brine then dried over Na₂SO₄ and concentrated to give 820 mg of crude material. To 156 mg (0.60 mmol) of this material was added 3 mL toluene then 162 mg (0.60 mmol) of ethyl 3-bromo-1H-indole-2-carboxylate, 235 mg (0.90 mmol) of PPh₃ and 180 uL (0.90 mmol) DIAD and the solution stirred at room temperature for 12 hrs. The solution was concentrated then purified by silica gel chromatography (12 grams of silica gel eluting with 0-10% EtOAc in hexanes over 45 minutes) to give 160 mg (51%) of ethyl 3-bromo-1-{[4′-(methoxycarbonyl)biphenyl-3-yl]methyl}-1H-indole-2-carboxylate as a clear glass: 1H NMR (400 MHz, CDCl₃) δ 8.07 (d, 2H, J=8.0 Hz), 7.74 (d, 1H, J=8.2 Hz), 7.58 (d, 2H, J=8.5 Hz), 7.47 (d, 1H, J=8.0 Hz), 7.39-7.30 (m, 4H), 6.98 (d, 1H, J=8.0 Hz), 5.83 (s, 2H), 4.39 (q, 2H, J=7.2 Hz), 3.93 (s, 3H), 1.38 (t, 3H, J=7.2 Hz); MS (APCI) m/z 494 (MH+)

### Intermediate 17: Ethyl 3-(4-tert-butylphenyl)-1-{[4′-(methoxycarbonyl)biphenyl-3-yl]methyl}-1H-indole-2-carboxylate

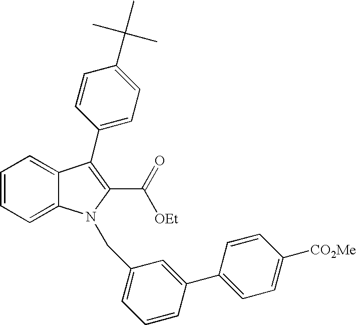

To a solution of 160 mg (0.33 mmol) of ethyl 3-bromo-1-{[4′-(methoxycarbonyl)biphenyl-3-yl]methyl}-1H-indole-2-carboxylate and 87 mg (0.49 mmol) of 4-tert-butylphenylboronic acid in 2.0 mL DME was added 8 mg Pd(PPh₃)₄ and 0.5 mL (0.98 mmol) of 2M Na₂CO₃ solution and the mixture stirred at 80° C. for 12 hrs. To the mixture was added 50 mL EtOAc then the solution was washed with 75 mL H₂O and 75 mL brine then dried over Na₂SO₄ and concentrated then purified by silica gel chromatography (12 grams of silica gel eluting with 0-10% EtOAc in hexanes over 45 minutes) to give 136 mg (77%) of ethyl 3-(4-tert-butylphenyl)-1-{[4′-(methoxycarbonyl)biphenyl-3-yl]methyl}-1H-indole-2-carboxylate as a clear glass: 1H NMR (400 MHz, CDCl₃). δ 8.08 (d, 2H, J=8.1 Hz), 7.64 (d, 1H, J=8.2 Hz), 7.58 (d, 2H, J=8.2 Hz), 7.51-7.35 (m, 7H), 7.28-7.25 (m, 1H), 7.19-71.2 (m, 2H), 6.78 (d, 1H, J=8.2 Hz), 5.88 (s, 2H), 4.17 (q, 2H, J=7.2 H), 3.97 (s, 3H), 1.42 (s, 9H), 0.98 (t, 3H, J=7.2 Hz); MS (APCI) m/z 546 (MH+)

### Intermediate 18a: Ethyl 1-{[4′-(benzyloxy)biphenyl-3-yl]methyl}-3-bromo-1H-indole-2-carboxylate

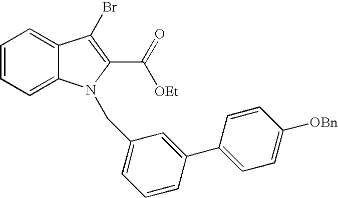

To a solution of 400 uL (3.43 mmol) of 3-bromobenzaldehyde and 940 mg (4.12 mmol) of 4-benzyloxyphenylboronic acid in 15 mL of DME was added 80 mg (0.07 mmol) Pd(PPh₃)₄ and 4.5 mL (8.58 mmol) of 2.0 M Na₂CO₃ (aq) and the mixture stirred at 90° C. for 3 hrs. To the cooled reaction was added 75 mL EtOAc and the solution was washed with 50 mL H₂O and 50 mL brine then dried over Na₂SO₄ and concentrated. To this residue was added 15 mL THF followed by 130 mg (3.43 mmol) of NaBH₄ and the solution stirred at room temperature for 4 hrs. Another 260 mg (6.86 mmol) of NaBH₄ was added and the mixture stirred for 12 hrs. The reaction was quenched with sat. NHCl₄ (aq) then extracted with two 50 mL portions of EtOAc. The combined organics were washed with 100 mL H₂O and 100 mL brine then dried over Na₂SO₄ and concentrated. To this residue in 9 mL toluene was added 680 mg (2.53 mmol) of 3-bromo-1H-indole-2-carboxylic acid, 1.0 g (3.80 mmol) PPh₃ and 750 uL DIAD then the solution was stirred at room temperature for 12 hrs. The solution was concentrated then purified by silica gel chromatography (40 grams of silica gel eluting with 0-10% EtOAc in hexanes over 45 minutes) and recrystallized from EtOAc and hexanes to give 360 mg (20%) of ethyl 1-{[4′-(benzyloxy)biphenyl-3-yl]methyl}-3-bromo-1H-indole-2-carboxylate as a white solid: 1H NMR (400 MHz, CDCl₃) δ 7.75 (d, 1H, J=8.2 Hz), 7.48-7.35 (m, 10H), 7.35-7.24 (m, 3H), 7.04 (d, 2H, J=8.5 Hz), 6.90 (d, 1H, J=7.8 Hz), 5.82 (s, 2H), 5.12 (s, 2H), 4.39 (q, 2H, J=7.0 Hz), 1.38 (t, 3H, J=7.0 Hz)

### Intermediate 18: Ethyl 1-{[4′-(benzyloxy)biphenyl-3-yl]methyl}-3-(4-tert-butylphenyl)-1H-indole-2-carboxylate

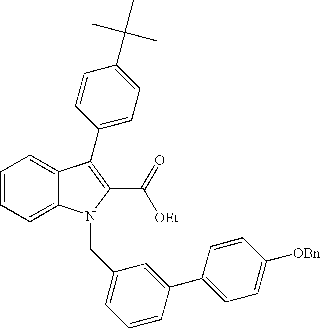

To 350 mg (0.65 mmol) of ethyl 1-{[4′-(benzyloxy)biphenyl-3-yl]methyl}-3-bromo-1H-indole-2-carboxylate in 5 mL of DME was added 173 mg (0.97 mmol) of 4-tert-butylphenylboronic acid, 15 mg Pd(PPh₃)₄ and 1.0 mL of 2.0 M Na₂CO₃ (aq) then stirred at 80° C. for 12 hrs. To this solution was added 75 mL EtOAc and the organics washed with 75 mL H₂O and 75 mL brine then dried over Na₂SO₄, concentrated and then purified by silica gel chromatography (40 grams of silica gel eluting with 0-10% EtOAc in hexanes over 45 minutes) and recrystallized from EtOAc and hexanes to give 310 mg (81%) of ethyl 1-{[4′-(benzyloxy)biphenyl-3-yl]methyl}-3-(4-tert-butylphenyl)-1H-indole-2-carboxylate as a clear glass: 1H NMR (400 MHz, CDCl₃) δ 7.62 (d, 1H, J=7.2 Hz), 7.47-7.26 (m, 16H), 7.16 (t, 1H, J=8.0 Hz), 7.05-6.97 (m, 3H), 5.84 (s, 2H), 5.18 (s, 2H), 4.12 (q, 2H, J=7.2 Hz), 1.38 (s, 9H), 0.97 (t, 3H, J=7.2 Hz); MS (ESI) m/z 594 (MH+)

### Intermediate 19a: [4′-(benzyloxy)biphenyl-3-yl]methyl methanesulfonate

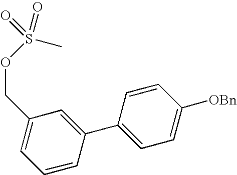

To 650 mg (2.25 mmol) of 4′-(benzyloxy)biphenyl-3-carbaldehyde in 10 mL THF was added 130 mg (3.38 mmol) NaBH₄ and the mixture stirred at room temperature for 2 hrs. The reaction was quenched with sat. NHCl₄ (aq) then extracted with two 50 mL portions of EtOAc. The combined organics were washed with 50 mL H₂O and 50 mL brine then dried over Na₂SO₄ and concentrated. This residue was taken up in 8 mL CH₂Cl₂, 470 uL (3.38 mmol) TEA and 210 uL (2.71 mmol) was added at 0° C. then the mixture was stirred at room temperature for 12 hrs. The solution was washed with two 25 mL portions of H₂O and 25 mL of brine then dried over Na₂SO₄, concentrated and purified by silica gel chromatography (40 grams of silica gel eluting with 0-20% EtOAc in hexanes over 45 minutes) and recystallized from EtOAc and hexanes to give 410 mg (50%) of [4′-(benzyloxy)biphenyl-3-yl]methyl methanesulfonate as a white solid: 1H NMR (400 MHz, CDCl₃). δ 7.58-7.32 (m, 11H), 7.08 (d, 2H, J=8.0 Hz), 5.12 (s, 2H), 4.65 (s, 2H), 3.66 (s, 3H)

### Intermediate 19: Ethyl 1-{[4′-(benzyloxy)biphenyl-3-yl]methyl}-3-(4-tert-butylphenyl)-1H-indole-2-carboxylate

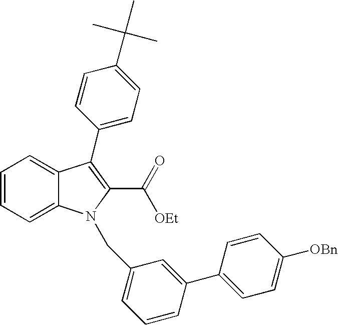

To 410 mg (1.12 mmol) of [4′-(benzyloxy)biphenyl-3-yl]methyl methanesulfonate and 240 mg (0.75 mmol) of ethyl 3-(4-tert-butylphenyl)-1H-indole-2-carboxylate in 5 mL DMF was added 260 mg (1.86 mmol) of K₂CO₃ and the mixture stirred at 50° C. for 12 hrs. Another 208 mg (1.49 mmol) K₂CO₃ added and the mixture stirred at 90° C. for 24 hrs. To the cooled solution was added 75 mL EtOAc and the mixture washed with three 75 mL portions of H₂O and 75 mL brine then dried over Na₂SO₄, concentrated and purified by silica gel chromatography (12 grams of silica gel eluting with 0-10% EtOAc in hexanes over 45 minutes) to give 295 mg (67%) of ethyl 1-{[4′-(benzyloxy)biphenyl-3-yl]methyl}-3-(4-tert-butylphenyl)-1H-indole-2-carboxylate as a white solid: 1H NMR (400 MHz, CDCl₃) δ 7.64 (d, 1H, J=8.1 Hz), 7.46-7.28 (m, 16H), 7.14 (t, 1H, 7.6 Hz), 7.05-6.99 (m, 3H), 5.86 (s, 2H), 5.10 (s, 2H), 4.10 (q, 2H, J=7.1 Hz), 1.38 (s, 9H), 0.95 (t, 3H, J=7.1 Hz)

### Intermediate 20a: Methyl 3′-formyl-4′-methylbiphenyl-4-carboxylate

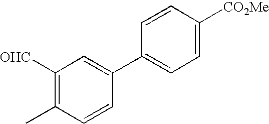

A solution of 1.5 g (7.54 mmol) of 5-bromo-2-methylbenzaldehyde, 1.88 g (11.3 mmol) of 4-carboxyphenylboronic acid, 170 mg Pd(PPh₃)₄, and 11.0 mL (22.6 mmol) of 2.0 M Na₂CO₃ (aq) in 35 mL DME was stirred at 80° C. for 12 hrs. The solution was filtered through a plug of Celite and silica then the solution was acidified with 1.0 N HCl (aq) and the resulting solids were collected by suction filtration, washed with H₂O and dried. To these solids was added 25 mL DMF, 560 uL (9.04 mmol) CH₃I, and 2.60 g (18.8 mmol) K₂CO₃ and the mixture stirred at room temperature for 2 hrs. To the mixture was added 150 mL EtOAc then was washed with three 100 mL portions of H₂O, 100 mL of brine then dried over Na₂SO₄ and purified by silica gel chromatography (40 grams of silica gel eluting with 0-30% EtOAc in hexanes over 45 minutes) to give 920 mg (48%) of methyl 3′-formyl-4′-methylbiphenyl-4-carboxylate as a white solid: 1H NMR (400 MHz, CDCl₃) δ 10.38 (s, 1H), 8.18 (d, 2H, J=7.8 Hz), 8.02 (s, 1H), 7.77 (d, 1H, J=7.8 Hz), 7.765 (d, 2H, J=8.0 Hz), 7.38 (d, 1H, J=8.0 H), 3.95 (s, 3H) 2.72 (s, 3H)

### Intermediate 20b: Methyl 3′-(hydroxymethyl)-4′-methylbiphenyl-4-carboxylate

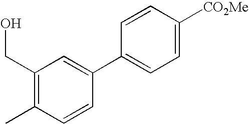

A solution of 920 mg (3.62 mmol) of methyl 3′-formyl-4′-methylbiphenyl-4-carboxylate and 205 mg (5.43 mmol) NaBH₄ in 15 mL THF was stirred at room temperature for 3 hrs. The reaction was quenched with sat. NHCl₄ (aq) then extracted with two 50 mL portions of EtOAc. The combined organics were washed with 75 mL H₂O and 75 mL brine then dried over Na₂SO₄. The solution was concentrated and the residue recrystallized from EtOAc and hexanes to yield 680 mg (73%) of methyl 3′-(hydroxymethyl)-4′-methylbiphenyl-4-carboxylate as a white solid: 1H NMR (400 MHz, CDCl₃). δ 8.80 (d, 2H, J=7.8 Hz), 7.65-7.61 (m, 3H), 7.45 (d, 1H, J=7.8 Hz), 7.26 (d, 1H, J=7.8 Hz), 4.77 (s, 2H), 3.95 (s, 3H), 2.39 (s, 3H)

### Intermediate 20: Ethyl 3-(4-tert-butylphenyl)-1-{[4′-(methoxycarbonyl)-4-methylbiphenyl-3-yl]methyl}-1H-indole-2-carboxylate

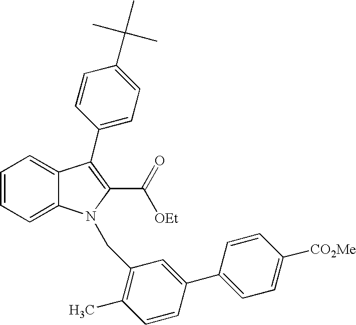

To a solution of 100 mg (0.39 mmol) of methyl 3′-(hydroxymethyl)-4′-methylbiphenyl-4-carboxylate in 3 mL CH₂Cl₂ was added 82 uL (0.58 mmol) TEA and 37 uL (0.47 mmol) MsCl and the solution stirred at room temperature for 12 hrs. To the solution was added 25 mL CH₂Cl₂ then the mixture was washed with 50 mL H₂O and 50 mL brine then dried over Na₂SO₄ and concentrated. The residue was taken up in 3 mL CH₃CN and 102 mg (0.32 mmol) of ethyl 3-(4-tert-butylphenyl)-1H-indole-2-carboxylate and 133 mg (0.96 mmol) K₂CO₃ was added then the mixture stirred at 80° C. for 12 hrs. To the mixture was added 100 mL EtOAc, then washed with 50 mL H₂O and 50 mL brine and dried over Na₂SO₄. The solution was concentrated and purified by silica gel chromatography (12 grams of silica gel eluting with 0-40% EtOAc in hexanes over 45 minutes) to give 105 mg (69%) of ethyl 3-(4-tert-butylphenyl)-1-{[4′-(methoxycarbonyl)-4-methylbiphenyl-3-yl]methyl}-1H-indole-2-carboxylate as a clear glass: 1H NMR (300 MHz, CDCl₃) δ 7.97 (d, 2H, J=8.3 Hz), 7.68 (d, 1H, J=8.0 Hz), 7.52-7.29 (m, 9H), 7.21-7.17 (m, 1H), 6.64 (s, 1H), 5.84 (s, 2H), 4.06 (q, 2H, J=7.1 Hz), 3.93 (s, 3H), 2.53 (s, 3H), 1.42 (s, 9H), 0.90 (t, 3H, J=7.0 Hz)

### Intermediate 21: Ethyl 3-(4-tert-butylphenyl)-1-{[4′-(2-ethoxy-1,1-dimethyl-2-oxoethoxy)-4-methylbiphenyl-3-yl]methyl}-1H-indole-2-carboxylate

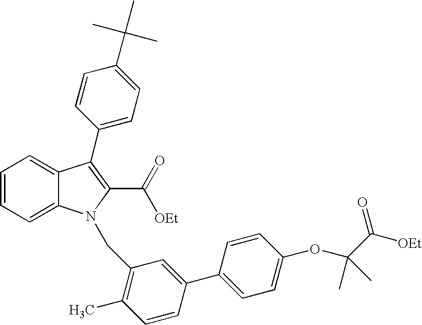

To a solution of 75 mg (0.15 mmol) of ethyl 3-(4-tert-butylphenyl)-1-[(4′-hydroxy-4-methylbiphenyl-3-yl)methyl]-1H-indole-2-carboxylate (intermediate 7) was added 1.0 mL of ethyl 2-bromo-2-methylpropanoate and 41 mg (0.30 mmol) of K₂CO₃ and the mixture stirred at 100° C. for 12 hrs. The solution was cooled and 50 mL EtOAc was added. The mixture was washed with 50 mL H₂O and 50 mL brine then dried over Na₂SO₄, concentrated and purified by silica gel chromatography (12 grams of silica gel eluting with 0-30% EtOAc in hexanes over 45 minutes) to give 66 mg (72%) of ethyl 3-(4-tert-butylphenyl)-1-{[4′-(2-ethoxy-1,1-dimethyl-2-oxoethoxy)-4-methylbiphenyl-3-yl]methyl}-1H-indole-2-carboxylate as a clear glass: 1H NMR (300 MHz, CDCl₃). δ 7.68 (d, 1H, J=8.0 Hz), 7.48-7.42 (m, 4H), 7.36-7.22 (m, 7H), 6.77 (d, 2H, J=7.6 Hz), 6.60 (s, 1H), 5.82 (s, 2H), 4.23 (q, 2H, J=7.0 Hz), 4.08 (q, 2H, J=7.0 Hz), 2.44 (s, 3H), 1.59 (s, 9H), 1.42 (s, 6H), 1.23 (t, 3H, J=7.0), 0.92 (t, 3H, J=7.0)

### Intermediate 22a: Ethyl 3-bromo-1-{[4′-(methoxycarbonyl)-4-methylbiphenyl-3-yl]methyl}-1H-indole-2-carboxylate

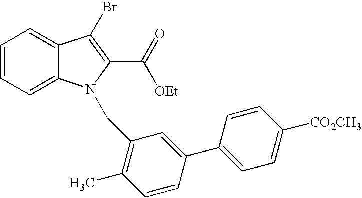

To 68.3 g (0.266 mol) of methyl 3′-(hydroxymethyl)-4′-methylbiphenyl-4-carboxylate in 1.0 L EtOAc was added at 10° C. 20.5 mL (0.280 mol) SOCl₂ and 1 mL pyridine. The solution was then stirred at room temperature for 12 hrs then washed with 500 mL 1.0 N HCl (aq), 500 mL sat. NaHCO₃ (aq) and 500 mL brine then dried over Na₂SO₄ and concentrated. To 750 mg (2.71 mmol) of this residue was added 660 mg (2.46 mmol) of ethyl 3-bromo-1H-indole-2-carboxylate in 8 mL DMF followed by 850 mg (6.16 mmol) K₂CO₃ and the mixture stirred at 70° C. for 4 hrs. The solution was cooled and 100 mL EtOAc was added. The solution was washed with three 25 mL portions of H₂O and 25 mL brine then dried over Na₂SO₄, concentrated, and purified by silica gel chromatography (120 grams of silica gel eluting with 0-20% EtOAc in hexanes over 45 minutes) to give 1.02 g (82%) of ethyl 3-bromo-1-{[4′-(methoxycarbonyl)-4-methylbiphenyl-3-yl]methyl}-1H-indole-2-carboxylate as white solid: 1H NMR (400 MHz, CDCl₃). δ 7.93 (d, 2H, J=8.5 Hz), 7.74 (d, 1H, J=7.9 Hz), 7.40-7.22 (m, 7H), 6.54 (s, 1H), 5.79 (s, 2H), 4.34 (q, 2H, J=7.4 Hz), 3.87 (s, 3H), 2.46 (s, 3H), 1.32 (t, 3H, J=7.4 Hz)

### Intermediate 22: Ethyl 3-(4-acetylphenyl)-1-{[4′-(methoxycarbonyl)-4-methylbiphenyl-3-yl]methyl}-1H-indole-2-carboxylate

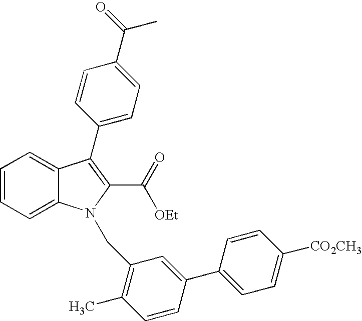

To 100 mg (0.20 mmol) of ethyl 3-bromo-1-{[4′-(methoxycarbonyl)-4-methylbiphenyl-3-yl]methyl}-1H-indole-2-carboxylate in 1.5 mL DMF and 0.5 mL H₂O was added 49 mg (0.30 mmol) 4-acetylphenylboronic acid, 10 mg 10% Pd/C and 50 mg (0.59 mmol) NaHCO₃ and the mixture stirred at 90° C. for 8 hrs. The mixture was filtered through a plug of Celite and silica gel with 50 mL EtOAc then the filtrate was washed with three 25 mL portions of H₂O and 25 mL brine and dried over Na₂SO₄, concentrated and purified by silica gel chromatography (12 grams of silica gel eluting with 0-20% EtOAc in hexanes over 45 minutes) to give 51 mg (47%) of ethyl 3-(4-acetylphenyl)-1-{[4′-(methoxycarbonyl)-4-methylbiphenyl-3-yl]methyl}-1H-indole-2-carboxylate as a white solid: 1H NMR (400 MHz, CDCl₃) δ 8.05 (d, 2H, J=8.0 Hz), 7.95 (d, 2H, J=8.0 Hz), 7.61 (d, 2H, J=8.2 Hz), 7.41-7.30 (m, 5H), 7.22-7.18 (m, 1H), 6.62 (s, 1H), 5.83 (s, 2H), 4.10 (q, 2H, J=7.2 Hz), 3.89 (s, 3H), 2.65 (s, 3H), 2.50 (s, 3H), 0.96 (t, 3H, J=7.2 Hz)

### Intermediate 23: 3′-[(Cyclopropylmethyl)oxy]-5′-({3-[4-(1,1-dimethylethyl)phenyl]-2-[(ethyloxy)carbonyl]-1H-indol-1-yl}methyl)-4-biphenylcarboxylic acid

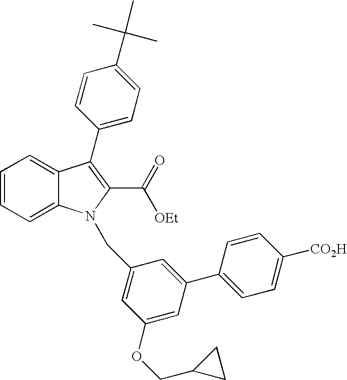

To 100 mg (0.20 mmol) of ethyl 3-(4-tert-butylphenyl)-1-[3-(cyclopropylmethoxy)-5-hydroxybenzyl]-1H-indole-2-carboxylate (Intermediate 1c) in 2 mL of CH₂Cl₂ was added at 0° C. 85 uL (0.50 mmol) Tf₂O and 84 uL (0.60 mmol) TEA. The solution was stirred at room temperature for 20 min then washed with 10 mL NaHCO₃ (aq), 10 mL H₂O and 10 mL brine then dried over Na₂SO₄ and concentrated. To this residue was added 46 mg (0.27 mmol) 4-carboxyphenylboronic acid, 5 mg Pd(PPh₃)₄ and 300 uL (0.55 mmol) of 2.0 M Na₂CO₃ in 1.5 mL DMF. The mixture was stirred at 90° C. for 3 hrs then cooled and filtered through a plug of Celite and silica gel with 50 mL EtOAc. The filtrate was washed with three 25 mL portions of H₂O and 25 mL of brine then dried over Na₂SO₄, concentrated and purified by silica gel chromatography (12 grams of silica gel eluting with 0-40% EtOAc in hexanes over 45 minutes) to give 59 mg (54%) of 3′-[(cyclopropylmethyl)oxy]-5′-({3-[4-(1,1-dimethylethyl)phenyl]-2-[(ethyloxy)carbonyl]-1H-indol-1-yl}methyl)-4-biphenylcarboxylic acid as a white foam: 1H NMR (400 MHz, CDCl₃) [symbol] 8.12 (d, 2H, J=8.2 Hz), 7.64-7.58 (m, 3H), 7.45-7.35 (m, 5H), 7.34 (t, 1H, J=6.9 Hz), 7.15 (t, 1H, J=7.7 Hz), 7.02 (s, 1H), 6.99 (s, 1H), 6.67 (s, 1H) 5.84 (s, 2H), 4.11 (q, 2H, J=7.1 Hz), 3.77 (d, 2H, J=7.0 Hz), 1.39 (s, 9H), 0.96 (t, 3H, J=7.1 Hz), 0.63-0.58 (m, 2H), 0.32-0.25 (m, 2H)

### Intermediate 24a: 3′-(Chloromethyl)-4-biphenylyl phenylmethyl ether

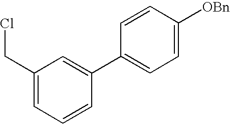

To 1.85 g (6.42 mmol) of 4′-[(phenylmethyl)oxy]-3-biphenylcarbaldehyde in 25 mL THF was added 290 mg (7.70 mmol) NaBH₄ and the mixture stirred at room temperature for 12 hr. To the mixture was added 75 mL EtOAc and washed with 25 mL sat. NH₄Cl, 25 mL H₂O and 25 mL brine then dried over Na₂SO₄. The organics were concentrated then the resulting residue was taken up in 25 mL EtOAc. The solution was cooled to 0° C. and 490 uL (6.74 mmol) of SOCl₂ and 5 drops of pyridine were added. The solution was stirred at room temperature for 2 hrs then washed with 25 mL 1.0 N HCl, 25 mL sat. NaHCO₃, and 25 mL brine then dried over Na₂SO₄ and concentrated to yield 1.21 g (61%) of 3′-(chloromethyl)-4-biphenylyl phenylmethyl ether as a white solid: 1H NMR (400 MHz, CDCl₃). δ 7.59-7.32 (m, 6H), 7.06 (d, 2H, J=8.7 Hz), 5.12 (s, 2H), 4.65 (s, 2H)

### Intermediate 24: Ethyl 3-[6-(methyloxy)-3-pyridinyl]-1-({4′-[(phenylmethyl)oxy]-3-biphenylyl}methyl)-1H-indole-2-carboxylate

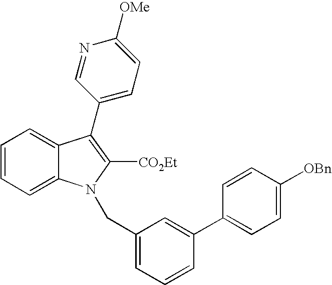

To 94 mg (0.30 mmol) of 3′-(chloromethyl)-4-biphenylyl phenylmethyl ether and 75 mg (0.25 mmol) of ethyl 3-[6-(methyloxy)-3-pyridinyl]-1H-indole-2-carboxylate in 1.0 mL DMF was added 105 mg (0.76 mmol) K₂CO₃ and the mixture stirred at 90° C. for 12 hr. To the mixture was added 25 mL EtOAc and then washed with three 25 mL portions of H₂O and 25 mL brine. The organics were then dried over Na₂SO₄, concentrated and purified by silica gel chromatography (12 grams of silica gel eluting with 0-20% EtOAc in hexanes over 45 minutes) to give 90 mg (63%) of ethyl 3-[6-(methyloxy)-3-pyridinyl]-1-({4′-[(phenylmethyl)oxy]-3-biphenylyl}methyl)-1H-indole-2-carboxylate as a clear glass: 1H NMR (400 Mhz, CDCl₃) δ 8.29 (s, 1H), 7.73 (d, 1H, J=8.5 Hz), 7.56 (d, 1H, J=7.8 Hz), 7.48-7.26 (m, 12H), 7.21-7.17 (m, 1H), 7.04-6.99 (m, 3H), 6.85 (d, 1H, J=7.4 Hz), 5.88 (s, 2H), 5.09 (s, 2H), 4.17 (q, 2H, J=7.0 Hz), 4.03 (s, 3H), 1.03 (t, 3H, J=7.0 Hz); MS (ESI) m/z 596 (MH+)

### Intermediate 25a: [6-(methyloxy)-3-pyridinyl]boronic acid

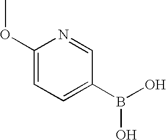

To a stirred solution of 17.0 mL (0.131 mol) of 5-bromo-2methoxypyridine in 130 mL THF at −78° C. was added 79 mL (0.197 mol) 2.5 M nBuLi (in hexanes) and the solution stirred for 2 min at −78° C. To this solution was added 45 mL (0.197 mol) of B(OiPr)₃ and the reaction allowed to warm to room temperature over 12 hr. The solution was then poured into 300 mL 1.0 N HCl (aq) and stirred vigorously for 30 min. The pH of the solution was raised to 7.0 with 3.0 N NaOH (aq) then the solution was extracted with three 150 mL portions of EtOAc. The combined organics were washed with 200 mL brine, dried over Na₂SO₄ and concentrated. This residue was then dissolved in 350 mL 2.0 M NaOH, washed with two 200 mL portions of EtOAc then the pH of the aqueous layer was lowered to 7.0 with conc. HCl (aq.) The resulting solids were filtered, washed with H₂O and dried to yield 15.01 g (75%) of [6-(methyloxy)-3-pyridinyl]boronic acid as a white powder: 1H NMR (400 MHz, DMSO-d6). δ 8.51 (s, 1H), 8.12 (bs, 2H), 7.95 (d, 1H, J=7.8 Hz), 6.73 (d, 1H, J=7.8 Hz), 3.83 (s, 3H)

### Intermediate 25: ethyl 3-[6-(methyloxy)-3-pyridinyl]-1H-indole-2-carboxylate

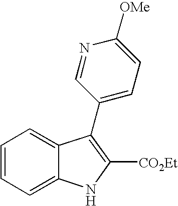

To a stirred solution of 7.0 g (26.2 mmol) of ethyl 3-bromo-1H-indole-2-carboxylate, 7.50 g (39.2 mmol) of [6-(methyloxy)-3-pyridinyl]boronic acid and 40 mL 2.0 M Na₂CO₃ (78.5 mmol) in 120 mL DME was added 1.0 g of Pd(PPh₃)₄ and the mixture stirred at 90° C. for 12 hr. The solution was filtered through a pad of Celite and the pad washed with 300 mL EtOAc. The combined organics were washed with 200 mL H₂O and 200 mL brine then dried over Na₂SO₄ and concentrated to yield 10.2 g (100%) of ethyl 3-[6-(methyloxy)-3-pyridinyl]-1H-indole-2-carboxylate as a tan solid: 1H NMR (400 MHz, CDCl₃). δ 9.52 (s, 1H), 8.38 (s, 1H), 7.78 (d, 1H, J=7.8 Hz), 7.75-7.60 (m, 3H), 7.58-7.55(m, 1H), 7.48-7.42 (m, 2H), 7.38-7.34 (m, 2H), 7.21-7.14 (m, 2H), 6.85-7.79 (m, 1H), 4.31 (q, 2H, J=7.0 Hz), 4.02 (s, 3H), 1.24 (t, 3H, J=7.0 Hz)

### Intermediate 26: Ethyl 3-{[3-(trifluoromethyl)phenyl]methyl}-1H-indole-2-carboxylate

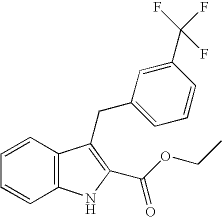

To a solution of 2-iodoaniline (5.4 g, 24.5 mmol) in DMF (40 mL) was added 3-trifluoromethylphenyl acetylene (5.0 g, 29.4 mmol), Et₂NH (15.2 mL, 146.9 mmol), CuI (93 mg, 0.5 mmol) and bis(triphenylphosphine)-palladium (II) acetate (183 mg, 0.24 mmol). The mixture was stirred at ambient temperature for 18 hours. The reaction was poured into saturated ammonium chloride (200 mL) and extracted with ether (2×150 mL). The combined ether was dried over magnesium sulfate and concentrated to afford 2-{[3-(trifluoromethyl)phenyl]ethynyl}aniline (intermediate 26a, 6.8 g) as a dark oil. Material used without further purification. 1H NMR (400 MHz, DMSO-d6): δ 8.03 (s, 1H), 7.86 (d, 1H), 7.69 (d, 1H), 7.61 (t, 1H), 7.23 (d, 1H), 7.08 (t, 1H), 6.71 (d, 1H), 6.51 (t, 1H), 5.65 (s, 2H); C15H10F3N1.

To a solution of the aniline (Intermediate 26a, 6.8 g) in THF (35 mL) at 5 C was added TFAA (6.8 mL, 49.0 mmol) over 20 minutes. The reaction was stirred for 1 hour, diluted with EtOAc (60 mL) followed by saturated NaHCO₃ (60 mL) and stirred for 30 minutes. The reaction was diluted with additional EtOAc (60 mL) and the layers separated. The EtOAc was washed with saturated NaHCO₃ (2×60 mL), dried over magnesium sulfate and concentrated. Purified by silica gel chromatography (5% EtOAc in Hexane) to afford 2,2,2-trifluoro-N-(2-{[3-(trifluoromethyl)phenyl]ethynyl}phenyl)acetamide (intermediate 26b , 6.25 g, 71% over two steps) as a light yellow solid. ¹H NMR (400 MHz, DMSO-d₆); δ 11.34 (s,1H), 7.79-7.70 (m, 3H), 7.69-7.66 (m, 2H), 7.54-7.40 (m, 3H); C₁₇H₉F₆N₁O₁.

To a solution of the acetamide (Intermediate 26b, 6.2 g, 17.4 mmol) in anhydrous DMSO (30 mL) was added ethyliodoacetate (5.6 g, 26.1 mmol) followed by K₂CO₃ (7.2 g, 52.2 mmol). The mixture was stirred at ambient temperature for 1 hour and then heated at 80^{˜} C for 6 hours. The mixture was poured into 1M NH₄Cl (200 mL) and extracted with ether (3×200 mL). The combined ether was dried over MgSO₄ and concentrated to an orange solid (6.4 g). Added hexane (60 mL) and stirred for 1 hour. The resulting solid was filtered, rinsed with hexane and dried to afford the title compound (Intermediate 26, 4.44 g, 74%) as a yellow solid. ¹H NMR (400 MHz, CDCl₃); δ 8.91 (br,1H), 7.61-7.57 (m, 2H), 7.42-7.40 (m, 3H), 7.33 (t, 2H), 7.13 (t, 1H), 4.55 (s, 2H), 4.42 (q, 2H), 1.36 (t, 3H); C₁₉H₁₆F₃N₁O₂.

### Intermediate 27: Ethyl 1-({3-[(cyclopropylmethyl)oxy]-5-hydroxyphenyl}methyl)-3-{[3-(trifluoromethyl)phenyl]methyl}-1H-indole-2-carboxylate

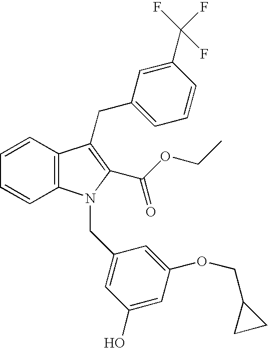

To a solution of 3,5-dihydroxybenzylalcohol (2.0 g, 14.3 mmol) and TEA (8.0 mL, 57.1 mmol) in THF (40 mL) at 5^{˜} C was added a solution of MsCl (5.7 g, 50.0 mmol) in THF (10 mL) over 30 minutes. Stirred for 1 hour. LiBr (6.2 g, 71.4 mmol) was added and the reaction was allowed to warm to ambient temperature and stir for 18 hours. The mixture was diluted with ether (100 mL) and washed with water (3×60 mL). The combined organics were dried over MgSO₄ to afford 5-(bromomethyl)benzene-1,3-diyl dimethanesulfonate (intermediate 27a , 5.1 g, quant.) as a light tan solid. Used without further purification. ¹H NMR (400 MHz, CDCl₃); δ 7.31 (d,2H), 7.18 (t, 1H), 4.44 (s, 2H), 3.19 (s, 6H); C₉H₁₁Br₁O₆S₂.

A mixture of the benzyl bromide Intermediate 27a (1.13 g, 3.2 mmol), Intermediate 26 (1.0 g, 2.9 mmol) and K₂CO₃ (796 mg, 5.8 mmol) in DMF (8 mL) was stirred at ambient temperature for 18 hours. The mixture was poured into water (60 mL) and extracted with ether (100 mL). The ether was washed with water (2×60 mL), brine (60 mL), dried over MgSO₄ and concentrated to afford ethyl 1-({3,5-bis[(methylsulfonyl)oxy]phenyl}methyl)-3-{[3-(trifluoromethyl)phenyl]methyl}-1H-indole-2-carboxylate (intermediate 27b , 1.78 g, 99%) as an orange paste. Used without further purification. ¹H NMR (400 MHz, DMSO-d₆); δ 7.76 (d, 1H), 7.64 (s, 1H), 7.58 (d, 1H), 7.50-7.43 (m, 3H), 7.35-7.28 (m, 2H), 7.14 (t, 1H), 6.97 (d, 2H), 5.86 (s, 2H), 4.55 (s, 2H), 4.20 (q, 2H), 3.35 (s, 6H), 1.08 (t, 3H); C₂₈H₂₆F₃N₁O₈S₂.

To a solution of the bis-mesylate Intermediate 27b (1.76 g, 2.8 mmol) in THF (15 mL) at 5^{˜} C was added TBAF (2.8 mL, 2.8 mmol, 1M in THF) over 30 minutes. The reaction was stirred at ambient temperature for 18 hours and then heated at 55^{˜} C for 2 hours. HPLC showed reaction ˜40% complete. The addition of an additional 1.5 equivalents of TBAF and heating at 55^{˜} C for 10 hours was used to drive the reaction to completion. The reaction was poured into 50% saturated NH₄Cl (60 mL) and extracted with ether (100 mL). The ether was washed with water (3×80 mL), dried over MgSO₄ and concentrated to afford ethyl 1-({3-hydroxy-5-[(methylsulfonyl)oxy]phenyl}methyl)-3-{[3-(trifluoromethyl)phenyl]methyl}-1H-indole-2-carboxylate (Intermediate 27c , 1.43 g, 93%) as an amber oil. Used without further purification. ¹H NMR (400 MHz, DMSO-d₆); [symbol] 9.88 (s, 1H), 7.76 (d, 1H), 7.62 (d, 1H), 7.55 (d, 1H), 7.50-7.43 (m, 3H), 7.32 (t, 1H), 7.13 (t, 1H), 6.55 (s, 1H), 6.49 (s, 1H), 6.25 (s, 1H), 5.74 (s, 2H), 4.54 (s, 2H), 4.21 (q, 2H), 3.26 (s, 3H), 1.11 (t, 3H); C₂₇H₂₄F₃N₁O₆S₁.

A mixture of the indole Intermediate 27c (1.4 g, 2.6 mmol), K₂CO₃ (707 mg, 5.1 mmol) and bromomethylcyclopropane (518 mg, 3.8 mmol) in DMF (12 mL) was stirred at 55^{˜} C for 3 hours. The mixture was poured into water (80 mL) and extracted with ether (3×80 mL). The combined ether was washed with brine, dried over MgSO₄ and concentrated to afford ethyl 1-({3-[(cyclopropylmethyl)oxy]-5-[(methylsulfonyl)oxy]phenyl}methyl)-3-{[3-(trifluoromethyl)phenyl]methyl}-1H-indole-2-carboxylate (Intermediate 27d, 1.38 g, 90%) as an orange paste. Used without further purification. ¹H NMR (400 MHz, DMSO-d₆); δ 7.76 (d, 1H), 7.60 (s, 1H), 7.55 (d, 1H), 7.48-7.43 (m, 3H), 7.30 (t, 1H), 7.13 (t, 1H), 6.74 (t, 1H), 6.58 (s, 1H), 6.38 (s, 1H), 5.78 (s, 2H), 4.54 (s, 2H), 4.22 (q, 2H), 3.68 (d, 2H), 3.28 (s, 3H), 1.13-1.09 (m, 4H), 0.49-0.47 (m, 2H), 0.24-0.18 (m, 2H); C₃₁H₃₀F₃N₁O₆S₁.

A solution of the indole Intermediate 27d (1.28 g, 2.1 mmol) and TBAF (6.3 mL, 6.3 mmol, 1M in THF) in THF (4 mL) was stirred at 55^{˜} C for 23 hours. The reaction was diluted with EtOAc (80 mL), washed with 50% saturated NH₄Cl (2×50 mL), brine (50 mL), dried over MgSO₄ and concentrated. Purified by silica gel chromatography (20% EtOAc in hexane) to afford the title compound (Intermediate 27, 750 mg, 68%) as a pale yellow solid. ¹H NMR (400 MHz, DMSO-d₆); δ 9.29 (s, 1H), 7.74 (d, 1H), 7.60 (s, 1H), 7.53-7.41 (m, 4H), 7.30 (t, 1H), 7.11 (t, 1H), 6.10 (t, 1H), 5.93 (d, 2H), 5.66 (s, 2H), 4.53 (s, 2H), 4.23 (q, 2H), 3.59 (d, 2H), 1.14 (t, 3H), 1.11-1.05 (m, 1H), 0.49-0.45 (m, 2H), 0.22-0.18 (m, 2H); C₃₀H₂₈F₃N₁O₄.

### Intermediate 28: 1-(Bromomethyl)-3-[(cyclopropylmethyl)oxy]-5-{[2-(methyloxy)ethyl]oxy}benzene

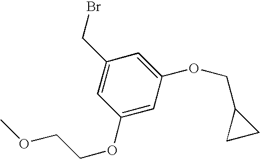

A mixture of 3,5-dihydroxybenzylalcohol (51.0 g, 0.36 mol) and K₂CO₃ (25.2 g, 0.18 mol) in DMF (200 mL) was stirred at 70^{˜} C for 45 minutes and then cooled to 50^{˜} C. A solution of cyclopropylmethylbromide (12.3 g, 0.09 mol) in DMF (20 mL) was added over 30 minutes and the mixture stirred at 50^{˜} C for 72 hours. The mixture was poured into water (600 mL), added concentrated HCl to pH˜7 and extracted with EtOAc (4×300 mL). The combined EtOAc was concentrated, taken up in 1N NaOH (400 mL) and extracted with ether (100 mL, discarded). The aqueous was cooled, added concentrated HCl to pH˜3 and extracted with ether (3×300 mL). The combined ether was dried over MgSO₄ and concentrated to afford 3-[(cyclopropylmethyl)oxy]-5-(hydroxymethyl)phenol (Intermediate 28a, 13.2 g, 75%) as a tan solid. ¹H NMR (400 MHz, DMSO-d₆); δ 9.23 (s, 1H), 6.29 (s, 1H), 6.26 (s, 1H), 6.12 (s, 1H), 5.04 (t, 1H), 4.32 (d, 2H), 3.69 (d, 2H), 1.19-1.11 (m, 1H), 0.53-0.49 (m, 2H), 0.28-0.24 (m, 2H); C₁₁H₁₄O₃.

A mixture of the phenol Intermediate 28a (13.1 g, 67.4 mmol), K₂CO ₃ (18.6 g, 134.9 mmol), bromoethylmethylether (24.4 g, 175.4 mmol) and 18-crown-6 (3.6 g, 13.6 mmol) in acetone (250 mL) was stirred at reflux for 20 hours. The mixture was concentrated, added water (400 mL) and extracted with ether (2×300 mL). The combined ether was washed with 1N NaOH (2×100 mL), brine (100 mL), dried over MgSO₄ and concentrated to afford (3-[(cyclopropylmethyl)oxy]-5-{[2-(methyloxy)ethyl]oxy}phenyl)methanol (Intermediate 28b, 16.5 g, 97%) as an orange oil. ¹H NMR (400 MHz, CDCl₃): δ 6.50 (s, 2H), 6.40 (t, 1H), 4.58 (s, 2H), 4.07 (t, 2H), 3.77-3.68 (m, 4H), 3.42 (s, 3H), 1.93 (s, 1H), 1.28-1.20 M, 1H), 0.64-0.59 (m, 2H), 0.32-0.29 M, 2H); C₁₄H₂₀O₄.

To a solution of the benzyl alcohol Intermediate 28b (17.3 g, 68.8 mmol) and TEA (14.3 mL, 102.9 mmol) in THF (120 mL) at 5^{˜} C was added a solution of MsCl (11.8 g, 102.9 mmol) in THF (30 mL) over 30 minutes. Reaction stirred at 5^{˜} C for 30 minutes and then at ambient temperature for 2 hours. Cooled to 5^{˜} C, added LiBr (31.6 g, 363.4 mmol) portionwise over 10 minutes and allowed to warm to ambient temperature and stir for 18 hours. The reaction was diluted with ether (400 mL), washed with water (2×150 mL), 0.5N NaOH (100 mL), brine (100 mL), dried over MgSO₄ and concentrated. Purified by silica gel chromatography (20% EtOAc in hexane) to afford the title compound (Intermediate 28, 16.6 g, 77%) as a colorless oil. ¹H NMR (400 MHz, CDCl₃); δ 6.54-6.52 (m, 2H), 6.41 (t, 1H), 4.38 (s, 2H), 4.08 (t, 2H), 3.75 (d, 2H), 3.72 (t, 2H), 3.43 (s, 3H), 1.29-1.19 (m, 1H), 0.65-0.58 (m, 2H), 0.38-0.30 (m, 2H); C₁₄H₁₉Br₁O₃.

### Intermediate 29: 1-(Chloromethyl)-3,5-bis{[2-(methyloxy)ethyl]oxy}benzene

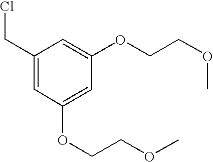

A mixture of 8.0 g methyl 3,5-dihydroxybenzoate in 150 mL DMF with 23 g K₂CO₃ and 16.5 g of bromoethylmethyl ether were stirred at 90° C. for 14 h. The rxn contents were filtered and the filtered solids washed with EtOAc. The combined solutions were poured into 100 mL of water and extracted 4× with 100 mL EtOAc. The organics were dried over MgSO₄, filtered, and concentrated. The resulting crude oil was flushed through a short pad of silica gel (˜1 inch pad on a 600 mL fritted glass funnel) eluting with hexanes followed by 20%-50% EtOAc in hexanes. Desired product fractions were isolated and concentrated to yield 13.23 (98%) grams of methyl 3,5-bis{[2-(methyloxy)ethyl]oxy}benzoate intermediate. To a solution of 13.2 g of methyl 3,5-bis{[2-(methyloxy)ethyl]oxy}benzoate in THF (200 mL) at 0-5° C. was added dropwise over 10 minutes 50 mL of 1.0 M LAH solution in THF. After 30 min at ˜5° C. the reaction was quenched with the slow addition of 1.9 mL H₂O, 1.9 mL of 1.0 N NaOH, and 5.7 mL of water. MgSO₄ was added, the mixture stirred for 10 minutes, then filtered and concentrated leaving 10.5 grams of a colorless oil of intermediate (3,5-bis{[2-(methyloxy)ethyl]oxy}phenyl)methanol. To a solution of 10.4 g of intermediate (3,5-bis{[2-(methyloxy)ethyl]oxy}phenyl)methanol in 200 mL of EtOAc at 0° C. was added 8.5 mL of DIEA (Hunigs base) followed by dropwise addition of 3.5 mL of MsCl. The solution was stirred for 60 min before adding 200 mg of solid KCl with warming to 50-60° C. for several hrs. The reaction was cooled to ambient temperature and the reaction mixture was washed with 0.1 N HCl and brine solutions. The organic phase was dried over Na₂SO₄, filtered, concentrated, and purified by silica gel chromatography (330 grams of silica gel eluting with 0-100% EtOAc in hexanes over 40 minutes). Product fractions were pooled and concentrated to yield 7.8 grams of the title compound (60% overall yield from starting compound methyl 3,5-dihydroxybenzoate). ¹H NMR (300 MHz, CDCl₃); δ 6.58 (d, 2H, J=2.2 Hz), 6.49 (t, 1H, J=2.2 Hz), 4.5 (s, 2H), 4.11 (m, 4H), 3.75 (m, 4H), 3.46 (t, 6H).

### Intermediate 30: Ethyl 3-(1λ⁵-diazynylidene)-3H-indole-2-carboxylate

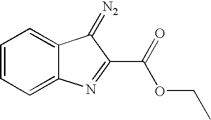

A solution of 5.00 g of ethyl indole-2-carboxylate in 500 ml of DCM sparged with nitrogen and maintained under a nitrogen atmosphere was treated with 18.23 g of NaNO₂ followed by 15 ml of glacial acetic acid added dropwise. The mixture was stirred at ambient temperature for 2 days then treated with 3.66 g of NaNO₂ and 3 ml of acetic acid and allowed to stir for one day. Approximately 300 ml of water was added to the mixture, and the organic phase was separated. The aqueous phase was made alkaline with sat. NaHCO₃, and extracted once with DCM. The combined organic phases were washed with sat. NaHCO₃, dried with Na₂SO₄ and concentrated in vacuo to give 5.59 g of yellow crystalline solid. The crude product was purified by chromatography on ˜150 g of silica gel eluting with 0-4% ethyl acetate/hexane to give 4.80 g of ethyl 3-(1λ⁵-diazynylidene)-3H-indole-2-carboxylate as a yellow crystalline solid. 1H NMR (DMSO-d6) δ 7.88 (m, 2H), 7.39 (m, 2H), 4.40 (q, 2H, J=7 Hz), 1.36 (t, 3H, J=7 Hz). MS ES+m/z 216 [M+H]+, 238 [M+Na]+. HPLC [Waters X-terra C-18; 10-100% CH3CN/H2O (0.1% TFA)/5 min; UV det.] RT=3.09 min (98%).

### Intermediate 31: Ethyl 1-[(3,5-bis{[2-(methyloxy)ethyl]oxy}phenyl)methyl]-3-[4-(1,1-dimethylethyl)phenyl]-1H-indole-2-carboxylate-

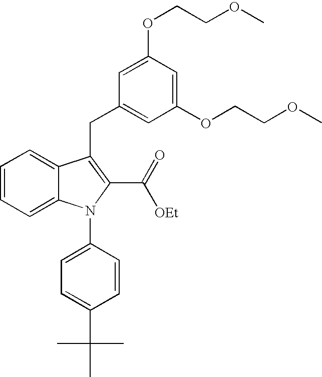

To a solution of 433 mg ethyl 3-[4-(1,1-dimethylethyl)phenyl]-1H-indole-2-carboxylate (WO 2002030895) and 500 mg of Intermediate 29 in 4.3 mL of DMF was added 564 mg of powdered K₂CO₃. The resulting suspension was heated to ˜100° C. over 90 minutes. The reaction mixture was cooled, poured into 20 mL EtOAc, washed with water (20 mL) and brine (20 mL), then dried over MgSO₄, filtered, and concentrated. The crude product was taken into several mL's of hot MeOH and set at ambient temperature overnight. The resulting solids were isolated by filtration and dried under vacuum at 60° C. for several hours to yield 706 mg of white solid Intermediate 31 (Ethyl 1-[(3,5-bis{[2-(methyloxy)ethyl]oxy}phenyl)methyl]-3-[4-(1,1-dimethylethyl)phenyl]-1H-indole-2-carboxylate). ¹H NMR (300 MHz, CDCl₃); δ 7.63 (d, 1H, J=8 Hz), 7.47 (d, 2H, J=8.4 Hz), 7.41 (d, 2H, J=8.4 Hz), 7.34 (m, 2H), 7.15 (m, 1H), 6.4 (d, 1H, J=2.2 Hz), 6.32 (d, 2H, J=1.9 Hz), 5.75 (s, 2H), 4.13 (q, 2H, J=7.1 Hz), 4.02 (m, 4H), 3.71 (m, 4H), 3.43 (s, 6H), 1.41 (s, 9H), 0.99 (t, 3H, J=7.2 Hz).

### Intermediate 32: Ethyl 3-[4-(1,1-dimethylethyl)phenyl]-1-({3-{[2-(methyloxy)ethyl]oxy}-5-[(phenylmethyl)oxy]phenyl}methyl)-1H-indole-2-carboxylate

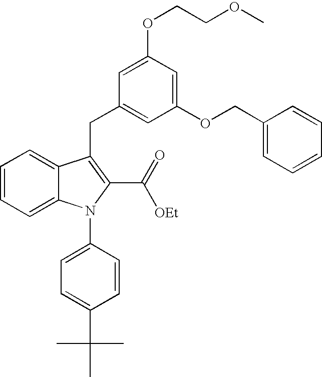

To 100 g of methyl 3,5-dihydroxybenzoate in DMF (500 mL) at 22° C. was added 173 g of powdered K₂CO₃ followed by 74.3 mL of benzyl bromide. Maintained stirring at ambient temperature for 24 hr, then added 1 L EtOAc, and 500 mL water (added 100 mL Et₂O to facilitate phase separation). The aqueous phase was extracted with EtOAc, the organics were dried (MgSO₄), filtered, and concentrated to an oil. The crude oil was taken into ˜200 mL EtOH (with heating) and set 72 hr in freezer. The solids that precipitated were filtered to give 30.9 grams of bis-alkylated product. The filtrate was concentrated and purified on 1 kg of silica gel eluting with hexanes followed by an EtOAc in hexanes gradient (5-30%). From the column was isolated 43 g of additional bis product and 49.49 grams (32% yield) of desired mono-benzylated product as Intermediate 32a (Methyl 3-hydroxy-5-[(phenylmethyl)oxy]benzoate) as a white solid; 1H NMR (300 MHz, CDCL3) d 7.3-7.46 (m, 6H), 7.22 (t, 1H, J=2.3 Hz), 7.26 (t, 1H, J=2.3 Hz), 6.73 (t, 1H, J=2.3 Hz), 6.3 (br s, 1H), 5.07 (s, 2H), 3.92 (s, 3H); LC/MS 257.20 (MH+, 100%).

To a solution of 4 g of Intermediate 32a in DMF (30 mL) was added K₂CO₃ (4.29 g) followed by 2.7 mL of bromoethylmethyl ether (Lancaster). The reaction was stirred vigorously at 90° C. for several hours (˜8 hr). TBME (60 mL) was added to the cooled mixture, the solids filtered (solids washed with 10 mL TBME), then 60 mL of 15% NaOH solution was added to the mixture. The aqueous phase was extracted with 20 mL TBME, the combined TBME solutions were dried (Na₂SO₄), filtered, and concentrated resulting in an isolation of 4.66 g of crude Intermediate 32b (Methyl 3-{[2-(methyloxy)ethyl]oxy}-5-[(phenylmethyl)oxy]benzoate)._This crude ester was taken into 80 mL THF, cooled to ˜0° C., added 16 mL 1.0 N LAH in THF solution, stirred 30 min, then slowly quenched cold with 0.6 mL water, 0.6 mL 1.0 N NaOH, and 1.8 mL water. Added MgSO₄, stirred 10 min, filtered, then concentrated to an oil that was purified by silica gel chromatography (120 gram column, 0-50% elution with EtOAc in hexanes to yield alcohol Intermediate 32c ({3-{[2-(methyloxy)ethyl]oxy}-5-[(phenylmethyl)oxy]phenyl}methanol).

Alcohol Intermediate 32c (3.9 g) in 60 mL of EtOAc was cooled to 0° C. and DIEA (2.83 mL) was added followed by the dropwise addition over several minutes of 1.15 mL of MsCl. After stirring 2.5 hr 100 mg of KCl solid was added and the mixture stirred with heating to 50° C. for 3 hr followed by cooling to ambient temperature with stirring overnight. Added 50 mL of water and 100 mL of EtOAc and the organic phase was washed with saturated NaHCO₃ (50 mL) and brine (50 mL). The organics were dried over Na₂SO₄, filtered, then concentrated to yield ˜4.2 g of crude intermediate 32d (1-(chloromethyl)-3-{[2-(methyloxy)ethyl]oxy}-5-[(phenylmethyl)oxy]benzene) as a viscous yellow-colored oil: 1H NMR (400 MHz, CDCl3) δ 7.43-7.28 (m, 5H), 6.63 (s, 1H), 6.58 (s, 1H), 6.52 (t, 1H, J=2.1 Hz), 5.03 (s, 2H), 4.5 (s, 2H), 4.1 (m, 2H), 3.73 (m, 2H), 3.44 (s, 3H).

To a solution of Intermediate 32d (3.6 g) and 3.0 g of ethyl 3-(4-tert-butylphenyl)-1H-indole-2-carboxylate in 60 mL of DMF was added 3.7 g of K₂CO₃ (powdered) and the resulting mixture stirred at ˜90° C. for 2.5 hr. before cooling. The reaction mixture was diluted into 50 mL water and 100 mL of EtOAc, washed with 50 mL of NaHCO₃ solution and 50 mL brine, then dried over Na₂SO₄, filtered, and concentrated to an oil. The crude oil was purified by silica gel chromatography to yield 4.2 g of a nearly colorless oil as Intermediate 32 (Ethyl 3-[4-(1,1-dimethylethyl)phenyl]-1-({3-{[2-(methyloxy)ethyl]oxy}-5-[(phenylmethyl)oxy]phenyl}methyl)-1H-indole-2-carboxylate). 1H NMR (400 MHz, CDCl3) δ 7.62 (d, 1H, 8Hz), 7.45 (d, 2H, J=8 Hz), 7.39 (d, 2H, J=8 Hz), 7.35-7.27 (m, 5H), 7.13 (m, 1H), 6.42 (t, 1H, J=2.2 Hz), 6.36 (s, 1H), 6.30 (s, 1H), 5.72 (s, 2H), 4.94 (s, 2H), 4.1 (q, 2H, J=7.1 Hz), 4.0 (t, 2H, J=4.7 Hz), 3.4 (s, 3H). 1.38 (s, 9H), 0.96 (t, 3H, J=7.1 Hz).

### Intermediate 33: 1-(chloromethyl)-3-{[2-(methyloxy)ethyl]oxy}-5-(trifluoromethyl)benzene

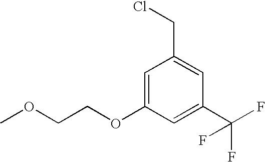

A solution of 2 g of 3-nitro-5-(trifluoromethyl)benzoic acid in 50 mL of EtOH was saturated with HCl (gas) for 1 min. and 10% Pd/C (100 mg) was added. Reaction mixture was stirred under atmospheric pressure of hydrogen (balloon) for 16 hrs. Catalyst was removed by filtration and solvent removed under reduced pressure.

1 g of product was dissolved in 5 mL of hot 35% sulfuric acid and then allowed to cool to below 15° C. Ice (5 g) was added and the amine bisulfate precipitated. A solution of 0.385 g (5.57 mmol) of NaNO₂ in 5 mL of ice water was added dropwise under the surface of the ice-cooled solution with stirring at such a rate as to maintain the temperature at 0-5° C. After the solution had been stirred for an additional 5 min, a few crystals of urea were added to decompose any excess NaNO₂. To the cold (0° C.) solution was added a solution of 15 g (62 mmol) of CuNO₃.3H₂O in 150 mL of water at room temperature. With vigorous stirring, 0.583 g (4.07 mmol) of CuO was added to the solution. The liquid became dark blue and rapidly changed to green. About 1 min after the addition of CuO the nitrogen evolution ceased and the reaction was complete. The mixture was extracted with ether and organic layers were combined and solvent evaporated yielding 0.45 g of product. Crude material was dissolved in 5.0 mL of DMF and 1.252 g of Cs₂CO₃ and 0.267 g (1.92 mM) of 1-bromo-2-(methyloxy)ethane were added and mixture stirred overnight. Reaction mixture was diluted then with water and product extracted with EtOAc providing 0.31 g of product. Ethyl 3-{[2-(methyloxy)ethyl]oxy}-5-(trifluoromethyl)benzoate (0.30 g) was dissolved in 5.0 ml of THF and 1.23 mL of 1M solution of LAH was added. Mixture was stirred for several hours and excess of LAH was decomposed by 1N solution of NaOH and inorganic solid filtered off. Solvent was removed under reduced pressure and 0.25 g of product was obtained. 0.15 g of crude product was dissolved in DCM and 0.086 g (0.72 mM) of SOCl₂ was added. Reaction mixture was stirred overnight and solvent removed under reduced pressure providing 0.15 g of Intermediate 33 (1-(chloromethyl)-3-{[2-(methyloxy)ethyl]oxy}-5-(trifluoromethyl)benzene).

### Intermediate 34: 1-(chloromethyl)-3-[(cyclopropylmethyl)oxy]-5-(trifluoromethyl)benzene

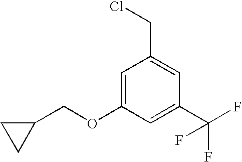

A solution of 2 g of 3-nitro-5-(trifluoromethyl)benzoic acid in 50 mL of EtOH was saturated with HCl (gas) for 1 min. and 10% Pd/C (100 mg) was added. Reaction mixture was stirred under atmospheric pressure of hydrogen (balloon) for 16 hrs. Catalyst was removed by filtration and solvent removed under reduced pressure.

1 g of product was dissolved in 5 mL of hot 35% sulfuric acid and then allowed to cool to below 15° C. Ice (5 g) was added and the amine bisulfate precipitated. A solution of 0.385 g (5.57 mmol) of NaNO₂ in 5 mL of ice water was added dropwise under the surface of the ice-cooled solution with stirring at such a rate as to maintain the temperature at 0-5° C. After the solution had been stirred for an additional 5 min, a few crystals of urea were added to decompose any excess NaNO₂. To the cold (0° C.) solution was added a solution of 15 g (62 mmol) of CuNO₃.3H₂O in 150 mL of water at room temperature. With vigorous stirring, 0.583 g (4.07 mmol) of CuO was added to the solution. The liquid became dark blue and rapidly changed to green. About 1 min after the addition of cuprous oxide the nitrogen evolution ceased and the reaction was complete. The mixture was extracted with ether and organic layers were combined and solvent evaporated yielding 0.45 g of product. Crude material (0.23 g; 0.98 mM) was dissolved in 5.0 mL of DMF 0.96 g of Cs₂CO₃ and 0.199 g (1.47 mM) of (bromomethyl)cyclopropane were added and mixture stirred overnight. Reaction mixture was diluted then with water and product extracted with EtOAc providing 0.25 g of product. Ethyl 3-[(cyclopropylmethyl)oxy]-5-(trifluoromethyl)benzoate (0.25 g) was dissolved in 5.0 ml of THF an 1.23 mL of 1M solution of LAH was added. Mixture was stirred for several hours and excess of LAH was decomposed by 1N solution of NaOH and inorganic solid filtered off. Solvent was removed under reduced pressure and 0.20 g of product was obtained. The crude product (0.15 g) was dissolved in DCM and 0.086 g (0.72 mM) of SOCl₂ was added. Reaction mixture was stirred overnight and solvent removed under reduced pressure providing 0.15 g of Intermediate 34 (1-(chloromethyl)-3-[(cyclopropylmethyl)oxy]-5-(trifluoromethyl)benzene). ¹H NMR (400 MHz, Chloroform-d): δ 7.20 (bs 1H); 7.10 (bs, 1H); 7.07 (bs, 1H); 4.56 (s, 2H); 3.84 (d, 2H): 1.32-1.23 (m, 1H); 0.70-0.64 (m,2H); 0.39-0.33 (m,2H).

### Intermediate 35 Methyl 3-[4-(1,1-dimethylethyl)phenyl]-1-{[3-{[2-(methyloxy)ethyl]oxy}-5-(4-morpholinyl)phenyl]methyl}-1H-indole-2-carboxylate

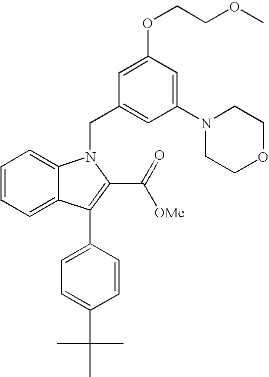

To a solution of 253 g (0.79 mol) of ethyl 3-(4-tert-butylphenyl)-1H-indole-2-carboxylate in 1.5 L of NMP was added 112 g (0.998 mol) of KOtBu over several minutes. The mixture was stirred at 32-35° C. over 1 hr, then 271.36 g of 3,5-dibromobenzyl bromide was added over 25 min keeping the temperature below 50° C. Stirred for 2.5 hr, then added 10 g of additional KOtBu followed by 15 g of additional tribromide. Stirred for 30 min at ambient temperature to give a crude solution of Intermediate 35a (Ethyl 1-[(3,5-dibromophenyl)methyl]-3-[4-(1,1-dimethylethyl)phenyl]-1H-indole-2-carboxylate).

To the crude solution of Intermediate 35a was added a solution of 53 g of KOH in 500 mL water over 5 min. The mixture was heated to 66° C., then the heat source was removed and the mixture stirred overnight. The mixture was reheated to 60° C. and the following added in succession; conc. HCl (50 mL), water (850 mL), NMP (409 mL), and conc. HCl (450 mL). Heated to 70° C., cooled to 60° C. and some gum-like material was collected with a spatula and triturated with CH₃CN. This solid was used as seed as the mixture was cooled slowly to 30° C. Stirred 1 hr at 30° C., solids isolated and dried at 70° C., then triturated in DCM. Isolated two additional crops of solid from the mother liquors, total isolated yield of 285 g of Intermediate 35b (1-[(3,5-Dibromophenyl)methyl]-3-[4-(1,1-dimethylethyl)phenyl]-1H-indole-2-carboxylic acid). 1H NMR (400 MHz, d₆-DMSO δ 13.03 (br s, 1H), 7.7 (s, 1H), 7.61 (d, 2H, J=8.6 Hz), 7.45 (m, 4H), 7.36 (m, 4H), 7.13 (t, 1H, J=7.5 Hz), 5.8 (s, 2H), 1.32 (s, 9H).

To a mixture of KOtBu (220 g) and DME (215 mL) was added 315 mL DMPU and then 188 mL of 2-methoxyethanol was added over 5 min. The mixture was stirred at 35° C. for 15 min, heated to 60° C., then a slurry of Intermediate 35b (283 g in 100 mL DMPU and 215 mL DME) was added. Heated jacket to 115° C. (removed by distillation 200 mL of DME) until mixture temperature reached 104° C., then refluxed 4 hrs, cooled. Diluted with water (750 mL) and slowly acidified with 6N HCl (500 mL). Extracted with EtOAc (3 L), washed the organics with water (2×1800 mL), then distilled off 2 L of EtOAc. Added 2 L of CH₃CN, then concentrated. Recrystallized from ˜500 mL hot CH₃CN cooling to 0° C. Solids were filtered, washed with 150 mL of CH₃CN, and dried at 55° C. in a vacuum oven to provide 147 g of Intermediate 35c (1-[(3-Bromo-5-{[2-(methyloxy)ethyl]oxy}phenyl)methyl]-3-[4-(1,1-dimethylethyl)phenyl]-1H-indole-2-carboxylic acid) as a sand-colored solid.

To a solution of 147 g of Intermediate 35c in 400 mL DCM and 14.4 mL of MeOH was added DMAP (5 g) and then EDCl.HCl (66 g) portionwise over 10 min. The mixture was stirred at ambient temperature for 2 hrs, partially concentrated to ˜¼ volume, then 1.2 L of EtOAc added and the solution was washed with 1N HCl (2×), water, 10% Na₂CO₃, and brine. The organics were dried over Na₂SO₄, filtered, concentrated, reconstituted into 1.6 L toluene, partially concentrated to ˜750 mL to provide a crude toluene solution of Intermediate 35d (Methyl 1-[(3-bromo-5-{[2-(methyloxy)ethyl]oxy}phenyl)methyl]-3-[4-(1,1-dimethylethyl)phenyl]-1H-indole-2-carboxylate).

To the crude solution of Intermediate 35d was added morpholine (30 mL) followed by BINAP (17 g), Cs₂CO₃ (170 g), and Pd(OAc)₂ (3.08 g). The mixture was heated to 100° C. for 1 hr before adding Pd₂dba₃.CHCL₃ (1.0 g). continued stirring at 100° C. for 2 hrs. Added an additional 0.5 g of Pd(OAc)₂ before stirring overnight at ˜100° C. Cooled, filtered the reaction mixture through 300 g silica gel washing with 1.2 L of EtOAc. The filtrate was concentrated, then taken into 900 mL THF and used as Intermediate 35 (Methyl 3-[4-(1,1-dimethylethyl)phenyl]-1-{[3-{[2-(methyloxy)ethyl]oxy}-5-(4-morpholinyl)phenyl]methyl}-1H-indole-2-carboxylate) as a crude solution in THF.

### Intermediate 36 Ethyl 3-[4-(1,1-dimethylethyl)phenyl]-1-[(3-{[2-(methyloxy)ethyl]oxy}-5-{[(trifluoromethyl)sulfonyl]oxy}phenyl)methyl]-1H-indole-2-carboxylate

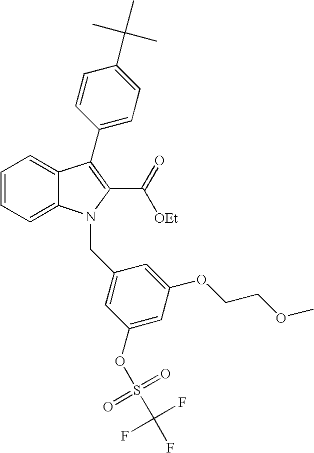

To a solution of 15.0 g (46.7 mmol) of ethyl 3-[4-(1,1-dimethylethyl)phenyl]-1H-indole-2-carboxylate (WO 2002030895) and 13.5 g (98.0 mmol) of K₂CO₃ in 100 mL DMF was added 20.1 g (56.0 mmol) of Intermediate 27a_(5-(bromomethyl)benzene-1,3-diyldimethanesulfonate). The mixture was stirred at room temperature for 16 hr then 350 mL EtOAc was added. The solution was washed with three 200 mL portions of H₂O then 200 mL of brine. After drying over 10 g of Na₂SO₄ the solution was concentrated to yield 30.9 g of Intermediate 36a (Ethyl 1-({3,5-bis[(methylsulfonyl)oxy]phenyl}methyl)-3-[4-(1,1-dimethylethyl)phenyl]-1H-indole-2-carboxylate) as a beige foam: 1H NMR (400 MHz, CDCl₃). δ 7.64 (d, 1H), 7.45 (d, 2H, J=8.5 Hz), 7.39-7.30 (m, 4H), 7.18-7.14 (m, 2H), 7.00 (s, 2H), 5.88 (s, 2H), 4.14-4.06 (m, 2H), 3.06 (s, 6H), 1.38 (s, 9H), 1.25 (t, 3H, J=7.1 Hz);

To 5.0 g (8.34 mmol) of Intermediate 36a in 50 mL of THF was added 25.0 mL of 1.0 M TBAF in THF. After stirring at 50° C. for 3 hr the solution was poured into 40 mL sat. NH₄Cl (aq.) The resulting mixture was extracted with 200 mL of Et₂O and the organics were washed with 100 mL of H₂O then 100 mL of brine. After drying over 2 g of Na₂SO₄ the solution was concentrated to yield 4.57 g of Intermediate 36b (Ethyl 3-[4-(1,1-dimethylethyl)phenyl]-1-({3-hydroxy-5-[(methylsulfonyl)oxy]phenyl}methyl)-1H-indole-2-carboxylate) as a beige foam: 1H NMR (400 MHz, CDCl₃). δ 7.63 (d, 1H, J=8.1 Hz), 7.45-7.42 (m, 2H), 7.38-7.30 (m, 4H), 7.17-7.13 (m, 1H), 6.65 (s, 1H), 6.61 (s, 1H), 6.44 (s, 1H), 5.73 (s, 2H), 4.08 (q, 2H, J=7.1 Hz), 3.02 (s, 3H), 1.37 (s, 9H), 0.95 (t, 3H, J=7.1 Hz)

To a solution of 3.57 g (6.84 mmol) of Intermediate 36b and 2.36 g (17.1 mmol) of K₂CO₃ in 20 mL of DMF was added 770 μL (8.21 mmol) of 2-bromoethyl methyl ether. After stirring at room temperature for 12 hr, another 320 μL (3.42 mmol) of 2-bromoethyl methyl ether was added and the mixture stirred at 60° C. for 4 hr. 150 mL of EtOAc was added and the solution washed with four 100 mL portions of H₂O and 100 mL of brine then the organics were concentrated. To this residue was added 50 mL of THF and 18 mL (17.6 mmol) of 1.0 M TBAF in THF. After stirring at room temperature for 16 hr, the solution was poured into 100 mL of sat. NH₄Cl (aq.) This mixture was extracted with 200 mL of EtOAc and the organics layer was then washed with 100 mL of H₂O and 100 mL of brine then concentrated and the residue purified by silica gel chromatography (120 grams of silica gel eluting with 0-40% EtOAc in hexanes over 45 minutes) to give 1.92 g (56%) of Intermediate 36c (Ethyl 3-[4-(1,1-dimethylethyl)phenyl]-1-[(3-hydroxy-5-{[2-(methyloxy)ethyl]oxy}phenyl)methyl]-1H-indole-2-carboxylate) as a white foam: 1H NMR (400 MHz, CDCl₃). δ 7.62 (d, 1H, J=8.1 Hz), 7.45 (d, 2H, J=8.4 Hz), 7.39-7.30 (m, 4H), 7.19-7.15 (m, 1H), 6.69-6.65 (m, 3H), 5.77 (s, 2H), 4.09 (q, 2H, J=7.1 Hz), 4.02-4.00 (m, 2H), 3.67 (m, 2H), 3.39 (s, 3H), 1.38 (s, 9H), 0.95 (t, 3H, J=7.1 Hz);

To a solution of 750 mg (1.50 mmol) of Intermediate 36c and 310 μL (2.24 mmol) of TEA in 8 mL of CH₂Cl₂ at 0° C. was added 280 μL (1.64 mmol) of trifluoromethanesulphonic anhydride. The resulting solution was stirred at room temperature for 30 minutes then washed with two 5 mL portions of H₂O and 5 mL of brine then concentrated. The residue was purified by silica gel chromatography (40 grams of silica gel eluting with 0-20% EtOAc in hexanes over 45 minutes) to give 510 mg (54%) of the title compound Intermediate 36 (Ethyl 3-[4-(1,1-dimethylethyl)phenyl]-1-[(3-{[2-(methyloxy)ethyl]oxy}-5-{[(trifluoromethyl)sulfonyl]oxy}phenyl)methyl]-1H-indole-2-carboxylate) as a viscous yellow oil: 1H NMR (400 MHz, CDCl₃). δ 7.63 (d, 1H, J=8.0 Hz), 7.46 (d, 2H, J=8.4 Hz), 7.38-7.29 (m, 4H), 7.18-7.14 (m, 1H), 6.69-6.65 (m, 3H), 5.77 (s, 2H), 4.09 (q, 2H, J=7.2 Hz), 4.07-4.03 (m, 2H), 3.69-3.66 (m, 2H), 3.40 (s, 3H), 1.39 (s, 9H), 0.95 (t, 3H, J=7.2 Hz); MS (ESI) m/z 634 (MH+)

### Examples

### Example 1

### 1-({3-[(cyclopropylmethyl)oxy]-5-[(phenylmethyl)oxy]phenyl}methyl)-3-[4-(1,1-dimethylethyl)phenyl]-1H-indole-2-carboxylic acid

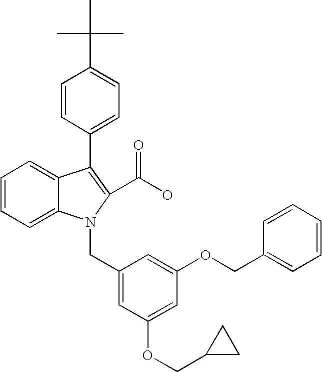

To a stirred solution of 50 mg (0.09 mmol) of ethyl 1-[3-(benzyloxy)-5-(cyclopropylmethoxy)benzyl]-3-(4-tert-butylphenyl)-1H-indole-2-carboxylate (see the synthesis of intermediate 1) in 2.0 mL THF and 1.0 mL MeOH was added 1.0 mL of 2.0M NaOH (aq) then the solution was stirred at 50° C. for 12 hrs. The solution was acidified with 1.0 N HCl (aq) and extracted with two 25 mL portions of EtOAc. The combined organics were washed with 50 mL H₂O and 50 mL brine then dried over Na₂SO₄, concentrated and purified by silica gel chromatography (12 grams of silica gel eluting with 0-40% EtOAc in hexanes over 45 minutes) to yield 18 mg (39%) of the title compound 1-[3-(benzyloxy)-5-(cyclopropylmethoxy)benzyl]-3-(4-tert-butylphenyl)-1H-indole-2-carboxylic acid as a tan foam: 1H NMR (400 MHz, CDCl₃). δ 7.60 (d, 1H, J=8.0 Hz), 7.55-7.39 (m, 4H), 7.38-7.22 (m, 7H), 7.118-7.11 (m, 1H), 6.38 (s, 1H), 6.31 (s, 1H), 6.27 (s, 1H), 5.75 (s, 2H), 4.92 (s, 2H), 3.66 (d, 2H, J=7.0 Hz), 1.38 (s, 9H), 1.22-1.18 (m, 1H), 0.60-0.56 (m, 2H), 0.29-0.25 (m, 2H); MS (APCI) m/z 560 (MH+)

### Example 2

### 1-[(3-[(cyclopropylmethyl)oxy]-5-{[2-(methyloxy)ethyl]oxy}phenyl)methyl]-3-[4-(1,1-dimethylethyl)phenyl]-1H-indole-2-carboxylic acid

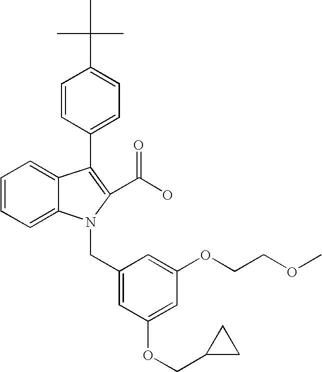

To a stirred suspension of 75 mg (0.15 mmol) of intermediate 1 and 52 mg (0.38 mmol) K₂CO₃ in 1.5 mL DMF was added 16 uL of 2-bromoethylmethyl ether and the mixture stirred at 50° C. overnight. To the cooled mixture was added 25 mL EtOAc and the solution washed with three 20 mL portions of H₂O, 20 mL brine then dried over Na₂SO₄ and concentrated. The residue was taken up in 2.0 mL THF and 1.0 mL MeOH, 1.0 mL 2.0 M NaOH (aq) was added and the solution stirred at 50° C. for 12 hrs. The cooled solution was acidified with 1.0 N HCl (aq), extracted with two 25 mL portions of EtOAc and the combined organics washed with brine then dried over Na₂SO₄ and purified by silica gel chromatography (12 grams of silica gel eluting with 0-40% EtOAc in hexanes over 45 minutes) to yield 18 mg (38%) of the title compound 3-(4-tert-butylphenyI)-1-[3-(cyclopropylmethoxy)-5-(2-methoxyethoxy)benzyl]-1H-indole-2-carboxylic acid as a white foam: 1H NMR (300 MHz, CDCl₃). δ 7.62 (d, 1H, J=5.1 Hz), 7.58-7.44 (m, 4H), 7.38 (s, 2H), 7.20-7.15 (m, 1H), 6.36 (s, 2H), 5.79 (s, 2H), 4.08-4.04 (m, 2H),. 3.72-3.65 (m, 4H), 3.42 (s, 3H), 1.41 (s, 9H), 1.23-1.17 (m, 1H), 0.95-0.90 (m, 2H), 0.65-0.58 (m, 2H); MS (APCI) m/z 528 (MH+)

### Example 3

### 1-({3-[(cyclopropylmethyl)oxy]-5-hydroxyphenyl}methyl)-3-[4-(1,1-dimethylethyl)phenyl]-1H-indole-2-carboxylic acid

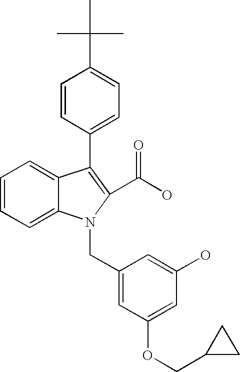

The title compound was obtained in 49% yield as white solid from Ethyl 3-(4-tert-butylphenyl)-1-[3-(cyclopropylmethoxy)-5-hydroxybenzyl]-1H-indole-2-carboxylate (Intermediate 1) as described in the synthesis of Example 1: 1H NMR (300 MHz, CDCl₃). δ 7.62 (d, 1H, J=8.0 Hz), 7.58-7.39 (m, 6H), 7.20-7.14 (m, 1H), 6.64 (s, 1H), 6.38 (s, 1H), 6.22 (s, 1H), 5.74 (s, 2H), 3.71 (d, 2H, J=6.9 Hz), 1.40 (s, 9H), 1.26-1.88 (m, 1H), 0.84-0.78 (m, 2H), 0.55-0.47 (m, 2H); MS (APCI) m/z 470 (MH+)

### Example 4

### 1-{[3-[(cyclopropylmethyl)oxy]-5-(methyloxy)phenyl]methyl}-3-[4-(1,1-dimethylethyl)phenyl]-1H-indole-2-carboxylic acid

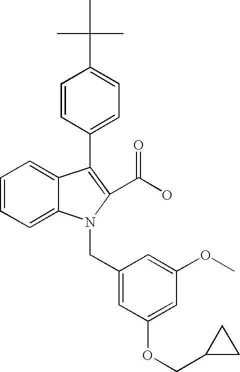

The title compound was obtained in 18% yield as a tan foam from ethyl 3-(4-tert-butylphenyl)-1-[3-(cyclopropylmethoxy)-5-hydroxybenzyl]-1H-indole-2-carboxylate (Intermediate 1) and CH₃I as described in the synthesis of Example 2: 1H NMR (400 MHz, CDCl₃). δ 7.58 (d, 1H, J=8.0 Hz), 7.49-7.40 (m, 4H), 7.36-7.29 (m, 2H), 7.32 (t, 1H, J=9.0 Hz), 6.30 (s, 1H), 6.24 (s, 2H), 5.76 (s, 2H), 3.69-3.65 (m 5H), 1.39 (s, 9H), 1.25-1.18 (m, 1H), 0.81-0.75 (m, 2H), 0.28-0.22 (m 2H); MS (APCI) m/z 484 (MH+)

### Example 5

### 1-({3,5-bis[(cyclopropylmethyl)oxy]phenyl}methyl)-3-[4-(1,1-dimethylethyl)phenyl]-1H-indole-2-carboxylic acid

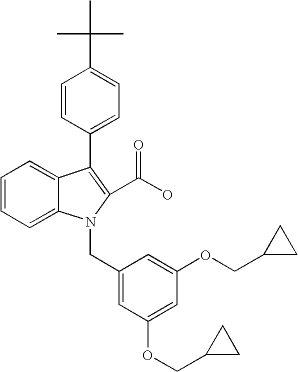

The title compound was obtained in 15% yield as an off-white foam from ethyl 3-(4-tert-butylphenyl)-1-[3-(cyclopropylmethoxy)-5-hydroxybenzyl]-1H-indole-2-carboxylate (intermediate 1) and (bromomethyl)cyclopropane as described in the synthesis of Example 2: 1H NMR (400 MHz, CDCl₃) δ 7.58 (d, 1H, J=8.0 Hz), 7.49-7.39 (m, 4H), 7.38-7.35 (m, 2H), 7.18-7.14 (m, 1H), 6.30 (s, 1H), 6.24 (s, 2H), 5.75 (s, 2H), 3.67 (d, 4H, J=7.0 Hz), 1.38 (s, 9H), 1.26-1.17 (m, 1H), 0.82-0.76 (m, 2H), 0.28-0.20 (m, 2H); MS (APCI) m/z 524 (MH+)

### Example 6

### 1-({3-[(cyclopropylmethyl)oxy]-5-[(3-methylbutyl)oxy]phenyl}methyl)-3-[4-(1,1-dimethylethyl)phenyl]-1H-indole-2-carboxylic acid

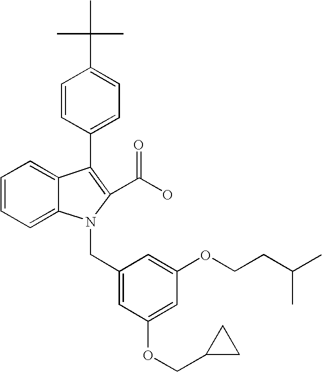

The title compound was obtained in 21% yield as a white foam from ethyl 3-(4-tert-butylphenyl)-1-[3-(cyclopropylmethoxy)-5-hydroxybenzyl]-1H-indole-2-carboxylate (intermediate 1) and 1-bromo-3-methylbutane as described in the synthesis of Example 2: 1H NMR (300 MHz, CDCl₃) δ 7.60 (d, 1H, J=8.1 Hz), 7.75-7.39 (m, 6H), 7.19-7.14 (m, 1H), 6.32 (s, 1H), 6.28 (s, 1H), 6.25 (s, 1H), 5.80 (s, 2H), 3.90 (t, 2H, J=6.8 Hz), 3.70 (d, 2H, J=6.9 Hz), 1.81-1.75 (m, 1H), 1.65-1.58 (m, 2H), 1.40 (s, 9H), 1.28-1.23 (m, 1H), 0.94 (d, 6H, J=6.5 Hz); MS (APCI) m/z 540 (MH+)

### Example 7

### 1-[(4′-carboxy-3-biphenylyl)methyl]-3-[4-(1,1-dimethylethyl)phenyl]-1H-indole-2-carboxylic acid

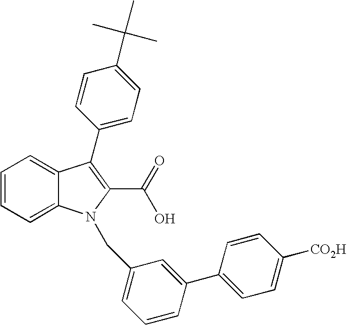

To 130 mg (0.24 mmol) of ethyl 3-(4-tert-butylphenyl)-1-{[4′-(methoxycarbonyl)biphenyl-3-yl]methyl}-1H-indole-2-carboxylate in 4 mL MeOH and 1.0 mL THF was added 1.0 mL 2.0 M NaOH (aq) then the mixture was stirred at 50° C. for 12 hrs. To the cooled solution was added 5 mL H₂O then the solution was extracted with two 25 mL portions of EtOAc. The combined organics were washed with 25 mL H₂O and 25 mL brine then dried over Na₂SO₄ and concentrated to yield 58 mg (48%) of the title compound as a white solid: 1H NMR (400 MHz, DMSO-d6), δ 13.00 (bs, 1H), 7.99 (d, 2H, J=8.2 Hz), 7.78-7.68 (m, 3H), 7.62-7.58 (m, 2H), 7.49-7.41 (m, 3H), 7.40-7.37 (m, 3H), 7.31 (t, 1H, J=7.6 Hz), 7.11 (t, 1H, J=7.4 Hz), 7.02 (d, 1H, J=7.6 Hz), 5.90 (s, 2H), 1.32 (s, 9H)

### Example 8

### 3-[4-(1,1-dimethylethyl)phenyl]-1-({4′-[(phenylmethyl)oxy]-3-biphenylyl}methyl)-1H-indole-2-carboxylic acid

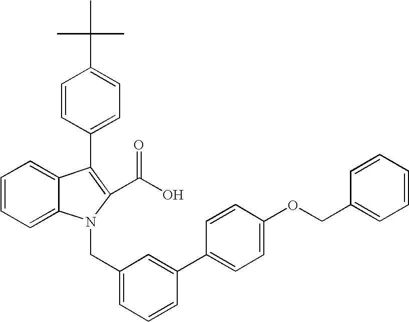

To 50 mg (0.08 mmol) of ethyl 1-{[4′-(benzyloxy)biphenyl-3-yl]methyl}-3-(4-tert-butylphenyl)-1H-indole-2-carboxylate in 4.0 mL THF and 1.0 mL MeOH was added 1.0 mL of 2.0 M NaOH (aq) and the solution stirred at 50° C. for 12 hrs. The cooled solution was acidified with 1.0 N HCl (aq) then extracted with two 25 mL portions of EtOAc. The combined organics were washed with 50 mL H₂O and 50 mL brine then dried over Na₂SO₄ and concentrated. The residue was recrystallized from EtOAc and hexanes to yield 35 mg (73%) of the title compound as a white solid: 1H NMR (400 MHz CDCl₃) δ 7.60 (d, 1H, J=8.1 Hz), 7.49-7.28 (m, 16H), 7.15 (t, 1H, J=7.4 Hz), 7.02-6.96 (m, 3H), 5.89 (s, 2H), 5.06 (s, 2H), 1.39 (s, 9H)

### Example 9

### 3-[4-(1,1-dimethylethyl)phenyl]-1-[(4′-hydroxy-3-biphenylyl)methyl]-1H-indole-2-carboxylic acid

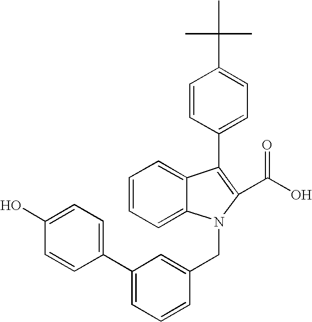

To 290 mg (0.75 mmol) of ethyl 1-{[4′-(benzyloxy)biphenyl-3-yl]methyl}-3-(4-tert-butylphenyl)-1H-indole-2-carboxylate in 3 mL of THF and 1 mL EtOH was added 0.5 mL of 2.0 M NaOH (aq) and the mixture stirred at 50° C. for 12 hrs. The cooled solution was acidified with 1.0 N HCl and extracted with two 25 mL portions of EtOAc. The combined organics were washed with 25 mL H₂O and 25 mL brine then dried over Na₂SO₄ and concentrated. To this residue was added 10 mL CHCl₃ and 2 mL MeOH followed by 20 mg 10% Pd/C. The mixture was stirred vigorously under 1 atm H₂ for 45 min. The solution was filtered through a pad of Celite and silica gel then dried over Na₂SO₄ and concentrated to give 135 mg (58%) of the title compound as a light gray solid: 1H NMR (400 MHz, DMSO-d6) δ 13.01 (s, 1H), 9.56 (s, 1H), 7.66 (d, 1H, J=7.6 Hz), 7.48-7.27 (m, 11H), 7.12 (t, 1H, J=7.6 Hz), 6.93 (d, 1H, J=6.6 Hz), 6.81 (d, 2H, J=6.8 Hz), 5.86 (s, 2H), 1.33 (s, 9H); MS (APCI) m/z 476 (MH+)

### Example 10

### 3-[4-(1,1-dimethylethyl)phenyl]-1-[(4′-hydroxy-4-methyl-3-biphenylyl)methyl]-1H-indole-2-carboxylic acid

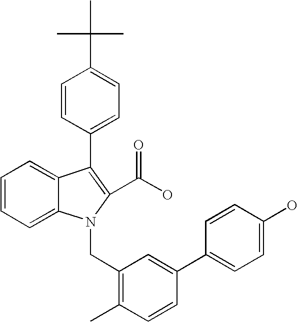

To 50 mg (0.10 mmol) of ethyl 3-(4-tert-butylphenyl)-1-[(4′-hydroxy-4-methylbiphenyl-3-yl)methyl]-1H-indole-2-carboxylate (intermediate 7) in 3.0 mL THF and 1.0 mL MeOH was added 1.0 mL of 2.0 M NaOH (aq) and the mixture stirred at 50° C. for 12 hrs. The cooled solution was acidified with 1.0 N HCl (aq) then extract with two 25 mL portions of EtOAc. The combines organics were washed with 25 mL H₂O and 25 mL brine then dried over Na₂SO₄ and concentrated to give 34 mg (72%) of the title compound as a white solid: 1H NMR (400 MHz, DMSO-d6), δ 12.84 (bs, 1H), 9.44 (s, 1H), 7.58-7.44 (m, 4H), 7.41 (d, 1H, J=8.1 Hz), 7.36-7.22 (m, 3H), 7.12 (t, 1H, J=7.4 Hz), 7.03 (d, 2H, J=8.2 Hz), 6.68 (d, 2H, J=8.2 Hz), 6.22 (s, 1H), 5.83 (s, 2H), 2.40 (s, 3H), 1.34 (s, 9H); MS (ESI) m/z 490 (MH+)

### Example 11

### 1-[(4′-carboxy-4-methyl-3-biphenylyl)methyl]-3-[4-(1,1-dimethylethyl)phenyl]-1H-indole-2-carboxylic acid

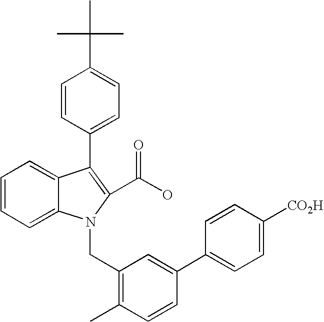

To 98 mg (0.17 mmol) of ethyl 3-(4-tert-butylphenyl)-1-{[4′-(methoxycarbonyl)-4-methylbiphenyl-3-yl]methyl}-1H-indole-2-carboxylate (Intermediate 20) in 3.0 mL THF and 1.0 mL H₂O was added 1.0 mL 2.0 M NaOH (aq) and the solution stirred at 50° C. for 12 hrs. The solution was cooled, acidified with 1.0 N HCl (aq) then extracted with two 25 mL portions of EtOAc. The combined organics were washed with 50 mL brine, dried over Na₂SO₄ then concentrated. The resulting residue was recrystallized from CH₂Cl₂, EtOAc and hexanes to give 23 mg (25%) of the title compound as a white solid: 1H NMR (400 MHz, DMSO-d6) 6 12.80 (bs, 1H), 7.86 (d, 2H, J=8.2 Hz), 7.58-7.38 (m, 7H), 7.35-7.24 (m, 4H), 7.12 (t, 1H, J=7.5 Hz), 6.38 (s, 1H) 5.86 (s, 2H), 2.46 (s, 3H), 1.36 (s, 9H); MS (ESI) m/z 518 (MH+)

### Example 12

### 1-({4′-[(1-carboxy-1-methylethyl)oxy]-4-methyl-3-biphenylyl}methyl)-3-[4-(1,1-dimethylethyl)phenyl]-1H-indole-2-carboxylic acid

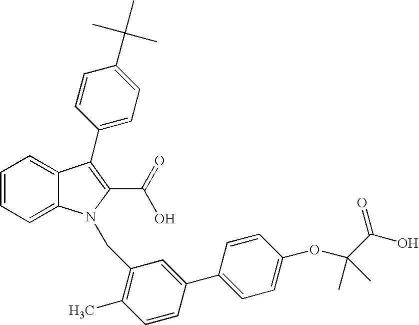

To 62 mg (0.10 mmol) of ethyl 3-(4-tert-butylphenyl)-1-{[4′-(2-ethoxy-1,1-dimethyl-2-oxoethoxy)-4-methylbiphenyl-3-yl]methyl}-1H-indole-2-carboxylate (intermediate 21) in 3 mL THF and 1 mL MeOH was added 1 mL 2.0 M NaOH (aq) and the solution stirred at 50° C. for 12 hrs. The solution was cooled, acidified with 1.0 N HCl (aq) then extracted with two 25 mL portions of EtOAc. The combined organics were washed with 50 mL brine, dried over Na₂SO₄ then concentrated. The resulting residue was recrystallized from CH₂Cl₂ and hexanes to give 41 mg (69%) of the title compound as a white solid: 1H NMR (400 MHz, DMSO-d6), δ 12.92 (bs, 1H), 7.68-7.59 (m, 4H), 7.40 (d, 2H, J=8.3 Hz), 7.36-7.25 (m, 3H), 7.18-7.12 (m, 3H), 6.74 (d, 2H, J=8.6 Hz), 6.27 (s, 1H), 5.83 (s, 2H), 2.41 (s, 3H), 1.47 (s, 6H), 1.37 (s, 9H); MS (ESI) m/z 576 (MH+)

### Example 13

### 3-[4-(1,1-dimethylethyl)phenyl]-1-{[4-methyl-4′-(methyloxy)-3-biphenylyl]methyl}-1H-indole-2-carboxylic acid

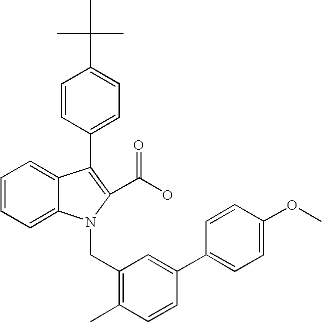

To 50 mg (0.10 mmol) of ethyl 3-(4-tert-butylphenyl)-1-[(4′-hydroxy-4-methylbiphenyl-3-yl)methyl]-1H-indole-2-carboxylate (intermediate 7) in 1.5 mL CH₃CN was added 12 uL (0.20 mmol) CH₃I and 34 mg (0.25 mmol) of K₂CO₃ then the mixture was stirred at room temperature for 12 hrs. To this mixture was added 0.5 mL DMF and stirring continued for 1 hr. Then 50 mL EtOAc was added and the solution washed with three 25 mL portions of H₂O and 25 mL of brine then dried over Na₂SO₄ and concentrated. This residue was then taken up in 3 mL THF and 1 mL MeOH and 1 mL 2.0 M NaOH (aq) was added then stirred at 50° C. for 12 hrs. The solution was cooled, acidified with 1.0 N HCl (aq) then extracted with two 25 mL portions of EtOAc. The combined organics were washed with 50 mL brine, dried over Na₂SO₄ then concentrated. The resulting residue was recrystallized from CH₂Cl₂ and hexanes to give 32 mg (67%) of the title compound as an off-white solid: 1H NMR (400 MHz, DMSO-d6), δ 7.58-7.48 (m, 4H), 7.41 (d, 2H, J=8.3 Hz), 7.38-7.26 (m, 3H), 7.19-7.10 (m, 3H), 6.87 (d, 2H, J=8.8 Hz), 6.26 (s, 1H), 5.83 (s, 2H), 3.69 (s, 3H), 2.42 (s, 3H), 1.37 (s, 9H); MS (ESI) m/z 504 (MH+)

### Example 14

### 3-(4-acetylphenyl)-1-[(4′-carboxy-4-methyl-3-biphenylyl)methyl]-1H-indole-2-carboxylic acid

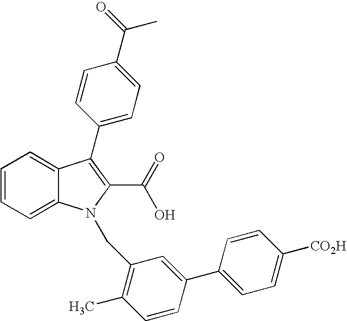

To 50 mg (0.09 mmol) of ethyl 3-(4-acetylphenyl)-1-{[4′-(methoxycarbonyl)-4-methylbiphenyl-3-yl]methyl}-1H-indole-2-carboxylate (Intermediate 22) in 2 mL THF and 1 mL MeOH was added 300 uL (0.55 mmol) of 2.0 M NaOH (aq) and the solution stirred at 60° C. for 2 hrs. Another 40 mg (1.00 mmol) NaOH added and the mixture stirred at 60° C. for 12 hrs. The solution was concentrated to ½ volume then poured into 15 mL 1.0 N HCl (aq). After 10 min, the resulting solids were collected by suction filtration, washed with 50 mL H₂O then dried to yield 31 mg (67%) of the title compounds as a pale yellow solid: 1H NMR (400 MHz, DMSO-d6). δ 12.92 (bs, 1H), 8.04 (d, 2H, J=8.0 Hz), 7.86 (d, 2H, J=8.1 Hz), 7.67-7.45 (m, 5H), 7.39-7.28 (m, 4H), 7.15 (t, 1H, J=7.5 Hz), 6.42 (s, 1H0, 5.48 (s, 2H), 2.62 (s, 3H), 2.42 (s, 3H); MS (ESI) m/z 504 (MH+)

### Example 15

### 1-({4′-carboxy-5-[(cyclopropylmethyl)oxy]-3-biphenylyl}methyl)-3-[4-(1,1-dimethylethyl)phenyl]-1H-indole-2-carboxylic acid

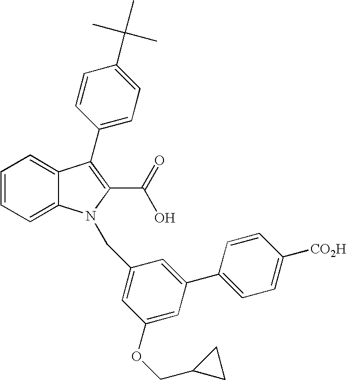

To 59 mg (0.10 mmol) of 3′-[(cyclopropylmethyl)oxy]-5-({3-[4-(1,1-dimethylethyl)phenyl]-2-[(ethyloxy)carbonyl]-1H-indol-1-yl}methyl)-4-biphenylcarboxylic acid in 2 mL THF and 1 mL MeOH was added 300 uL (0.59 mmol) of 2.0 M NaOH (aq) and the mixture stirred at 55° C. for 12 hrs. The solution was concentrated to ½ volume then 5 mL of 1.0 N HCl (aq) was added with vigorous stirring. After 5 min, the resulting solids were collected by suction filtration, washed with 10 mL H₂O and dried to yield 37 mg (66%) of the title compound as a beige solid: 1H NMR (400 MHz, DMSO-d6), δ 13.04 (bs, 1H), 7.96 (d, 2H, J=8.2 Hz), 7.72-7.68 (m, 3H), 7.52-7.40 (m, 5H), 7.38 (t, 1H, J=6.9 Hz), 7.14-7.07 (m, 3H), 6.59 (s, 1H), 5.82 (s, 2H), 3.80 (d, 2H, J=6.9 Hz), 1.37 (s, 9H), 1.20-1.15 (m, 1H), 0.58-0.52 (m, 2H), 0.28-0.22 (m, 2H); MS (ESI) m/z 574 (MH+)

### Example 16

### 1-[(4′-hydroxy-3-biphenylyl)methyl]-3-[6-(methyloxy)-3-pyridinyl]-1H-indole-2-carboxylic acid

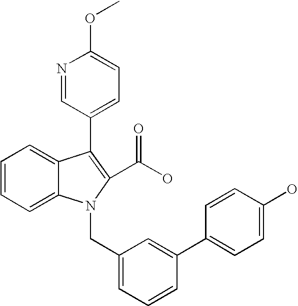

To 90 mg (0.16 mmol) of ethyl 3-[6-(methyloxy)-3-pyridinyl]-1-({4′-[(phenylmethyl)oxy]-3-biphenylyl}methyl)-1H-indole-2-carboxylate (Intermediate 24) in 2.0 mL THF and 1.0 mL H₂O was added 250 uL (0.47 mmol) of 2.0 M NaOH (aq) and the solution stirred at 60° C. for 12 hr. The solution was concentrated to ½ volume then added dropwise to 5 mL 1.0 N HCl. The solution was extracted with 20 mL EtOAC and the organics washed with 20 mL H₂O and 20 mL brine then dried over Na₂SO₄ and concentrated. The residue was taken up in 2 mL MeOH and 2 mL CH₂Cl₂, 10 mg Pd/C (10%, Degussa type) was added and the mixture stirred vigorously under 1 atm H₂ at room temperature for 5 hr. The solution was filtered through a plug of Celite and concentrated to yield 61 mg (86%) of the title compound as a tan foam: 1H NMR (400 MHz, DMSO-d6), δ 8.21 (s, 1H), 7.78 (d, 1H, J=7.3 Hz), 7.67 (d, 1H, J=7.3 Hz), 7.45-7.32 (m, 7H), 7.12 (t, 1H, J=7.5 Hz), 6.90 (d, 2H, J=8.6 Hz), 6.80 (d, 2H, J=8.6 Hz), 5.90 (s, 2H), 3.92 (s, 3H); MS (ESI) m/z 451 (MH+)

### Example 17

### 3-[4-(1,1-dimethylethyl)phenyl]-1-{[4-methyl-3′-(methylthio)-3-biphenylyl]methyl}-1H-indole-2-carboxylic acid

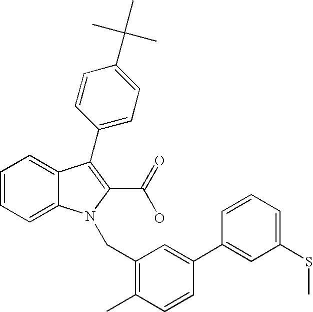

To 150 mg (0.30 mmol) of ethyl 1-(5-bromo-2-methylbenzyl)-3-(4-tert-butylphenyl)-1H-indole-2-carboxylate (intermediate 10) in 1.5 mL DME was added 75 mg (0.45 mmol) [3-(methylthio)phenyl]boronic acid, 7 mg (0.02 mmol) Pd(PPh₃)₄ and 450 uL (0.89 mmol) 2.0 M Na₂CO₃ (aq) then the mixture was stirred at 80° C. for 12 hr. The solution was filtered through a plug of Celite and the plug washed with 20 mL EtOAc. The combined organics were washed with 20 mL H₂O and 20 mL brine then concentrated and purified by silica gel chromatography (12 grams of silica gel eluting with 0-30% EtOAc in hexanes over 45 minutes.) The fractions containing product were concentrated. The residue was taken up in 1 mL EtOH, 2 mL THF and 1 mL H₂O, 80 mg (2.00 mmol) of NaOH was added and the solution stirred at 50° C. for 12 hr. The solution was concentrated to ½ volume, added dropwise to 5 mL 1.0 N HCl and the resulting solids were filtered, washed with H₂O and dried to yield 60 mg (40%) of the title compound as a light pink solid: 1H NMR (400 MHz, CDCl₃) δ 7.59 (d, 1H, J=8.1 Hz), 7.42-7.20 (m, 10H), 6.53 (s, 1H), 5.77 (s, 2H), 2.40 (s, 3H), 2.38 (s, 3H), 1.34 (s, 9H); MS (ESI) m/z 519 (MH+)

### Example 18

### 1-{[4′-carboxy-5-(methyloxy)-3-biphenylyl]methyl}-3-[4-(1,1-dimethylethyl)phenyl]-1H-indole-2-carboxylic acid

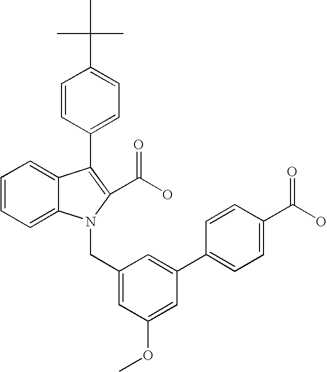

To 80 mg (0.14 mmol) of ethyl 3-(4-tert-butylphenyl)-1-{[5-hydroxy-4′-(methoxycarbonyl)biphenyl-3-yl]methyl}-1H-indole-2-carboxylate (intermediate 11) in 1.5 mL DMF was added 10 uL (0.21 mmol) CH₃I and 60 mg (0.43 mmol) K₂CO₃ and the mixture stirred at room temperature for 12 hr. To the mixture was added 25 ml EtOAc and washed with three 20 mL portions of H₂O and 25 mL brine then dried over Na₂SO₄, concentrated and purified by silica gel chromatography (12 grams of silica gel eluting with 0-20% EtOAc in hexanes over 45 minutes.) The fractions containing product were concentrated and the residue hydrolyzed as in Example 17 to yield 50 mg (67%) of the title compound as a white solid: 1H NMR (400 MHz, DMSO-d6). δ 12.98 (bs, 1H), 7.97 (d, 2H, J=8.3 Hz), 7.69-7.62 (m, 3H), 7.48-7.34 (m, 6H), 7.18-7.14 (m, 3H), 6.60 (s, 1H), 5.85 (s, 2H), 3.73 (s, 3H), 1.32 (s, 9H); MS (ESI) m/z 534 (MH+)

### Example 19

### 1-({4′-carboxy-5-[(phenylmethyl)oxy]-3-biphenylyl}methyl)-3-[4-(1,1-dimethylethyl)phenyl]-1H-indole-2-carboxylic acid

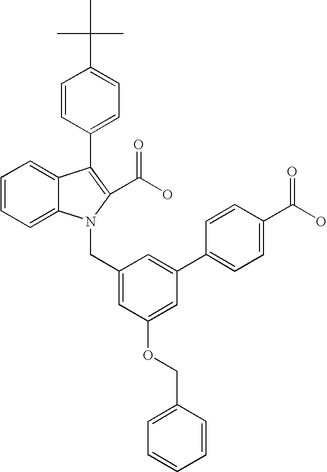

The title compound was obtained in 62% yield as a tan solid using ethyl 3-(4-tert-butylphenyl)-1-{[5-hydroxy-4′-(methoxycarbonyl)biphenyl-3-yl]methyl}-1H-indole-2-carboxylate (intermediate 11) and benzyl bromide as described for the synthesis of Example 18: 1H NMR (300 MHz, CDCl₃). δ 7.92 (d, 2H, J=8.4 Hz), 0.76-7.65 (m, 4H), 7.58-7.42 (m, 6H), 7.40-7.18 (m, 7H), 6.76 (s, 1H), 5.84 (s, 2H), 4.96 (s, 2H), 1.41 (s, 9H); MS (ESI) m/z 610 (MH+)

### Example 20

### 1-[(4′-carboxy-5-{[(methyloxy)methyl]oxy}-3-biphenylyl)methyl]-3-[4-(1,1-dimethylethyl)phenyl]-1H-indole-2-carboxylic acid

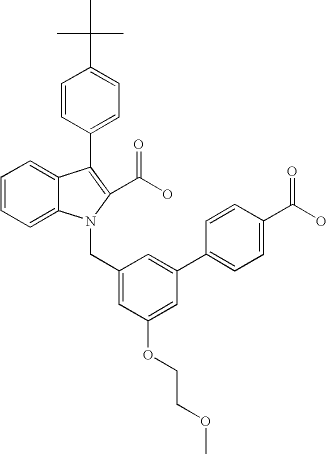

The title compound was obtained in 64% yield as a white solid using ethyl 3-(4-tert-butylphenyl)-1-{[5-hydroxy-4′-(methoxycarbonyl)biphenyl-3-yl]methyl}-1H-indole-2-carboxylate (intermediate 11) and bromoethylmethyl ether as described for the synthesis of Example 18: 1H NMR (300 MHz, CDCl₃) δ 7.92 (d, 2H, J=8.3 Hz), 7.75-7.62 (m, 4H), 7.55-7.42 (m, 5H), 7.23-7.17 (m, 2H), 7.06 (s, 1H), 6.93 (s, 1H), 5.84 (s, 2H), 4.40 (t, 2H, J=4.3 Hz), 3.68 (t, 2H, J=4.3 Hz), 3.46 (s, 3H), 1.40 (s, 9H); MS (ESI) m/z 578 (MH+)

### Example 21

### 1-[(4′-carboxy-4-methyl-3-biphenylyl)methyl]-3-[6-(methyloxy)-3-pyridinyl]-1H-indole-2-carboxylic acid

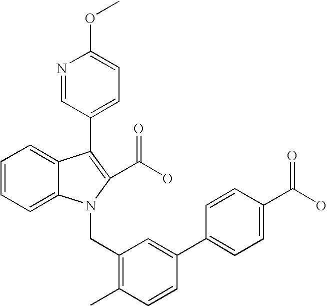

To 75 mg (0.25 mmol) of ethyl 3-[6-(methyloxy)-3-pyridinyl]-1H-indole-2-carboxylate (Intermediate 25) and 105 mg (0.76 mmol) K₂CO₃ in 1.0 mL DMF was added 84 mg (0.30 mmol) of methyl 3′-(chloromethyl)-4′-methyl-4-biphenylcarboxylate and the mixture stirred at 100° C. for 3 hr. To the mixture was added 25 mL EtOAc and then washed with three 25 mL portions of H₂O and 25 mL brine then concentrated and purified by silica gel chromatography (12 grams of silica gel eluting with 0-15% EtOAc in hexanes over 45 minutes.) The fractions containing product were concentrated and the residue hydrolyzed as in Example 17 to yield 49 mg (39%) of the title compound as a pale orange solid: 1H NMR (400 MHz, DMSO-d6), δ 12.90 (bs, 2H), 8.25 (s, 1H), 7.86 (d, 2H, J=8.5 Hz), 7.81 (d, 1H, J=8.4 Hz), 7.57 (d, 1H, J=8.4 Hz), 7.52-7.42 (m, 2H), 7.38-7.28 (m, 4H), 7.15 (t, 1H, J=7.5 Hz), 6.92 (d, 1H, J=7.5 Hz), 6.41 (s, 1H), 5.89 (s, 2H), 3.90 (s, 3H), 2.44 (s, 3H)

### Example 22

### 1-[(4′-carboxy-5-hydroxy-3-biphenylyl)methyl]-3-[4-(1,1-dimethylethyl)phenyl]-1H-indole-2-carboxylic acid

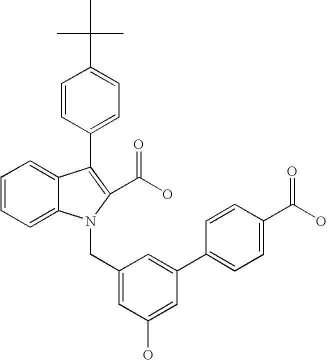

The title compound was obtained in 75% yield as a tan solid by hydrolyzing ethyl 3-(4-tert-butylphenyl)-1-{[5-hydroxy-4′-(methoxycarbonyl)biphenyl-3-yl]methyl}-1H-indole-2-carboxylate (Intermediate 11) as in Example 17: 1H NMR(300 MHz, DMSO-d6), δ 9.63 (s, 1H), 8.00 (d, 2H, J=8.4 Hz), 7.67 (d, 3H, J=8.4 Hz), 7.52-7.30 (m, 6H), 7.14 (t, 1H, J=7.5 Hz), 7.06 (s, 1H), 6.93 (s, 1H), 6.42 (s, 1H), 5.84 (s, 2H), 1.35 (s, 9H); MS (ESI) m/z 520 (MH+)

### Example 23

### 3-[4-(1,1-dimethylethyl)phenyl]-1-{[4′-(methylthio)-3-biphenylyl]methyl}-1H-indole-2-carboxylic acid

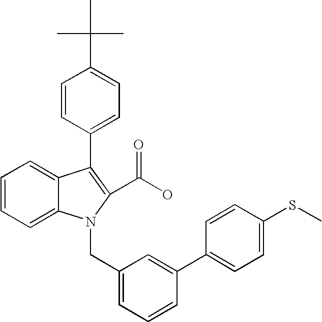

To 150 mg (0.31 mmol) of ethyl 1-[(3-bromophenyl)methyl]-3-[4-(1,1-dimethylethyl)phenyl]-1H-indole-2-carboxylate (intermediate 3), 460 uL (0.92 mmol) of 2.0 M Na₂CO₃ (aq), and 77 mg (0.46 mmol) of [4-(methylthio)phenyl]boronic acid in 1.5 mL DME was added 10 mg Pd(PPh₃)₄ and the mixture stirred at 80° C. for 12 hr. The mixture was then filtered through a plug of Celite and the plug washed with 25 mL EtOAc. The combined organics were washed with 25 mL H₂O and 25 mL brine then concentrated and purified by silica gel chromatography (12 grams of silica gel eluting with 0-25% EtOAc in hexanes over 45 minutes.) The fractions containing product were concentrated and the residue hydrolyzed as in Example 17 to yield 54 mg (35%) of the title compound as a tan solid: 1H NMR (400 MHz, DMSO-d6) δ 7.66 (s, 1H, J=8.6 Hz), 7.55-7.42 (m, 7H), 7.42-7.29 (m, 6H), 7.10 (t, 1H, J=7.5 Hz), 6.98 (d, 1H, J=7.5 Hz), 5.88 (s, 2H), 1.32 (s, 9H); MS (ESI) m/z 505 (MH+)

### Example 24

### 3-[4-(1,1-dimethylethyl)phenyl]-1-{[4′-(methylsulfonyl)-3-biphenylyl]methyl}-1H-indole-2-carboxylic acid

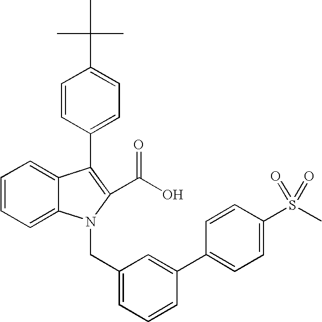

To 34 mg (0.07 mmol) of 3-[4-(1,1-dimethylethyl)phenyl]-1-{[4′-(methylthio)-3-biphenylyl]methyl}-1H-indole-2-carboxylic acid (Example 23) in 1.5 mL acetone and 0.5 mL H₂O was added 87 mg (0.14 mmol) of oxone and the mixture stirred at room temperature 12 hr. The mixture was filtered through a pad of Celite and the pad washed with 25 mL EtOAc. The combined organics were washed with 25 mL H₂O and 25 mL brine then dried over Na₂SO₄ and concentrated to yield 36 mg (100%) of the title compound as a tan foam: 1H NMR (400 MHz, CDCl₃). δ 7.95 (d, 2H, J=8.3 Hz), 7.69-7.59 (m, 3H), 7.51-7.38 (m, 9H), 7.18-7.10 (m, 2H), 5.92 (s, 3H), 3.05 (s, 3H), 1.39 (s, 9H); MS (ESI) m/z 538 (MH+)

### Example 25

### 3-[4-(1,1-dimethylethyl)phenyl]-1-{[3′-(methylsulfonyl)-3-biphenylyl]methyl}-1H-indole-2-carboxylic acid

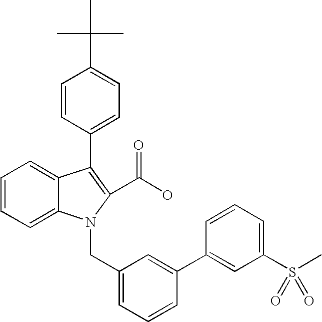

The title compound was obtained in 92% yield from 3-[4-(1,1-dimethylethyl)phenyl]-1-{[4-methyl}-4′-(methylthio)-3-biphenylyl]methyl}-1H-indole-2-carboxylic acid (intermediate 3) and [3-(methylthio)phenyl]boronic acid as described for the synthesis of Example 23 and Example 24: 1H NMR (400 MHz, CDCl₃). δ 8.08 (s, 1H), 7.89 (d, 1H, J=7.9 Hz), 7.76 (d, 1H, J=7.9 Hz), 7.61-7.55 (m, 2H), 7.52-7.34 (m, 9H), 7.19-7.10 (m, 2H), 5.93 (s, 2H), 3.06 (s, 3H), 1.39 (s, 9H); MS (ESI) m/z 560 (M+Na, 100%)

### Example 26

### 3-[4-(1,1-dimethylethyl)phenyl]-1-{[3-(4-morpholinyl)phenyl]methyl}-1H-indole-2-carboxylic acid

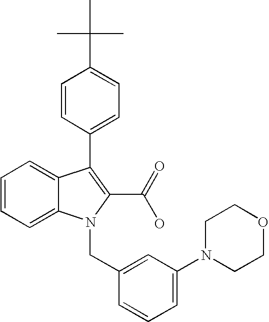

To a solution of 125 mg (0.25 mmol) of ethyl 1-(3-bromobenzyl)-3-(4-tert-butylphenyl)-1H-indole-2-carboxylate (intermediate 3), 67 uL (0.76 mmol) morpholine, 20 uL of tri-tertbutylphosphine (10% in hexanes), and 2 mg Pd(OAc)₂ in 1.5 mL toluene was added 98 mg (1.02 mmol) of NaOtBu and the mixture stirred at 50° C. for 2 hr. Another 2 mg Pd(OAc)₂ and 20 uL P(t-Butyl)₃ were added and the solution stirred at 50° C. for 12 hr. The mixture was filtered through a pad of Celite and the pad washed with 25 mL EtOAc. The combined organics were washed with 25 mL H₂O and 25 mL brine then concentrated and purified by silica gel chromatography (12 grams of silica gel eluting with 0-10% EtOAc in hexanes over 45 minutes.) The fractions containing product were concentrated and the residue hydrolyzed as in Example 17 to yield 25 mg (21%) of the title compound as a tan solid: 1H NMR (400 MHz, DMSO-d6), δ 7.57 (d, 1H, J=8.2 Hz), 7.51-7.30 (m, 5H), 7.26 (t, 1H, J=7.3 Hz), 7.10-7.02 (m, 2H), 6.84 (s, 1H), 6.77 (d, 1H, J=8.2 Hz), 6.44 (d, 1H, J=7.3 Hz), 5.72 (s, 2H), 3.77-3.65 (m, 4H), 3.07-2.96 (m, 4 h), 1.32 (s, 9H); MS (ESI) m/z 469 (MH+)

### Example 27

### 3-[4-(1,1-dimethylethyl)phenyl]-1-{[2-methyl-5-(4-morpholinyl)phenyl]methyl}-1H-indole-2-carboxylic acid

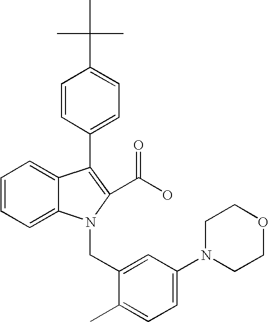

The title compound was obtained in 24% yield from ethyl 1-(5-bromo-2-methylbenzyl)-3-(4-tert-butylphenyl)-1H-indole-2-carboxylate (intermediate 10) and morpholine as described for the synthesis of Example 26: 1H NMR (400 MHz, DMSO-d6) δ 7.52-7.44 (m, 4H), 7.37 (d, 2H, J=8.2 Hz), 7.37-7.32 (m, 2H), 7.11 (t, 1H, J=7.7 Hz), 7.02 (d, 1H, J=8.4 Hz), 6.67 (d, 1H, J=8.6 Hz), 5.73 (s, 2H), 5.66 (s, 1H), 3.61-3.54 (m, 4H), 2.76-2.69 (m, 4H), 2.27 (m, 3H), 1.33 (s, 9H); MS (ESI) m/z 483 (MH+)

### Example 28

### 1-({4′-[(dimethylamino)carbonyl]-4-methyl-3-biphenylyl}methyl)-3-[4-(1,1-dimethylethyl)phenyl]-1H-indole-2-carboxylic acid

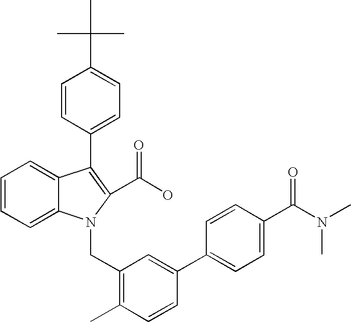

To a solution of 60 mg (0.10 mmol) of 3′-{[2-[(benzyloxy)carbonyl]-3-(4-tert-butylphenyl)-1H-indol-1-yl]methyl}-4′-methylbiphenyl-4-carboxyl is acid (intermediate 9), 23 mg (0.12 mmol) of EDCl and 16 mg (0.12 mmol) HOBt was added 200 uL of N,N-dimethylamine (2.0 M in THF) and the solution stirred at room temperature for 1 hr. The mixture was poured into 10 mL 1.0 N HCl (aq) and the resulting solids collected by suction filtration, washed with H₂O and dried. To this solid was added 3 mL CHCl₃, 1 mL MeOH and 5 mg Pd/C (10%, Degussa type) and the mixture stirred under 1 atm H₂ for 2 hr. The solution was filtered through a pad of Celite and concentrated to yield 21 mg (39%) of the title compound as a tan foam: 1H NMR (400 MHz, DMSO-d6). δ 12.89 (bs, 1H), 7.59-7.27 (m, 13H), 7.11 (t, 1H, J=7.4 Hz), 6.35 (s, 1H), 5.84 (s, 2H), 2.92 (bs, 3H), 2.85 (bs, 3H), 2.42 (s, 3H), 1.37 (s, 9H); MS (ESI) m/z 545 (MH+)

### Example 29

### 3-[4-(1,1-dimethylethyl)phenyl]-1-({4-methyl-3′-[(methylamino)carbonyl]-3-biphenylyl}methyl)-1H-indole-2-carboxylic acid

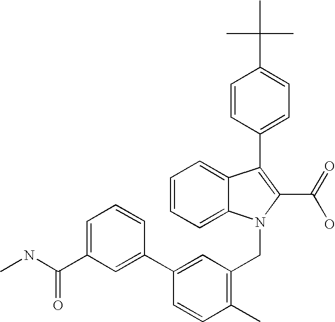

The title compound was obtained in 34% overall yield as a tan solid from 3′-[(3-[4-(1,1-dimethylethyl)phenyl]-2-{[(phenylmethyl)oxy]carbonyl}-1H-indol-1-yl)methyl]-4′-methyl-3-biphenylcarboxylic acid (Intermediate 12) and methylamine (2.0M in THF) as described for the synthesis of Example 28: 1H NMR (400 MHz, DMSO-d6) 6 12.85 (bs, 1H), 8.43 (bs, 1H), 7.79-7.75 (m, 1H), 7.65 (d, 1H, J=7.5 Hz), 7.521-7.24 (m, 11H), 7.09 (t, 1H, J=7.3 Hz), 6.45 (s, 1H), 5.84 (s, 2H), 2.71 (s, 3H), 2.44 (s, 3H), 1.37(s, 9H); MS (ESI) m/z 531 (MH+)

### Example 30

### 3-[4-(1,1-dimethylethyl)phenyl]-1-[(4-methyl-3′-{[(2-thienylmethyl)amino]carbonyl}-3-biphenylyl)methyl]-1H-indole-2-carboxylic acid

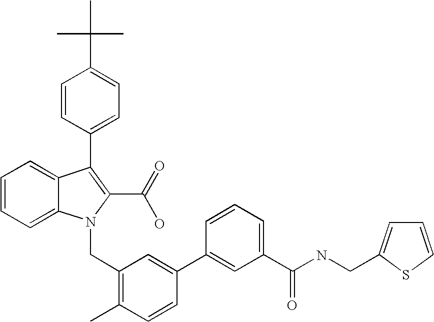

To a solution of 100 mg (0.17 mmol) of 3′-({2-{[(1,1-dimethylethyl)oxy]carbonyl}-3-[4-(1,1-dimethylethyl)phenyl]-1H-indol-1-yl}methyl)-4′-methyl-3-biphenylcarboxylic acid (Intermediate 14) in 1.0 mL DMF was added 40 mg (0.21 mmol) of EDCl and 29 mg (0.21 mmol) HOBt and the solution stirred at room temperature for 1 hr. To this solution was then added 35 uL (0.35 mmol) (2-thienylmethyl)amine and the mixture stirred at room temperature for 12 hr. 25 mL EtOAc was added and the solution washed with three 20 mL portions of H₂O and 20 mL brine then concentrated and purified by silica gel chromatography (12 grams of silica gel eluting with 0-20% EtOAc in hexanes over 45 minutes.) The fractions containing product were concentrated, the residue taken up in 1.5 mL CH₂Cl₂ and 0.50 mL TFA added. After stirring at 0° C. for 2.5 hr, 20 mL CH₂Cl₂ was added and the solution was washed with 15 mL sat. NaHCO₃ (aq), dried over Na₂SO₄, concentrated and purified by silica gel chromatography (12 grams of silica gel eluting with 0-60% EtOAc in hexanes over 45 minutes) to yield 13 mg (12%) of the title compound as an off-white solid: 1H NMR (400 MHz, CDCl₃). δ 7.63-7.55 (m, 3H), 7.48-7.44 (m, 4H), 7.34-7.23 (m, 6H), 7.14-7.11 (m, 2H), 6.92 (t, 1H, J=5.1 Hz), 6.79 (s, 1H), 6.52 (s, 1H), 6.18 (t, 1H, J=6.6 Hz), 5.81 (s, 2H), 3.54 (q, 2H, J=6.4 Hz), 3.03 (t, 2H, J=6.6 Hz), 2.43 (s, 3H), 1.37 (s, 9H)

### Example 31

### 3-[4-(1,1-dimethylethyl)phenyl]-1-{[4-methyl-3′-({[2-(2-thienyl)ethyl]amino}carbonyl)-3-biphenylyl]methyl-1H-indole-2-carboxylic acid

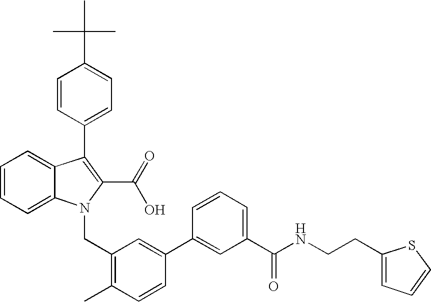

The title compound was obtained in 16% overall yield as a white foam from 3′-({2-{[(1,1-dimethylethyl)oxy]carbonyl}-3-[4-(1,1-dimethylethyl)phenyl]-1H-indol-1-yl}methyl)-4′-methyl-3-biphenylcarboxylic acid (Intermediate 14) and [2-(2-thienyl)ethyl]amine as described for the synthesis of Example 30: 1H NMR (400 MHz, CDCl₃) δ 7.60-7.44 (m, 8H), 7.32-7.26 (m, 5H), 7.20-7.11 (m, 4H), 6.91-6.88 (m, 1H), 6.50 (s, 1H), 6.38-6.34 (m, 1H), 5.80 (s, 2H), 4.64 (d, 2H, J=6.7 Hz), 2.41 (s, 3H), 1.38 (s, 9H)

### Example 32

### 3-[4-(1,1-dimethylethyl)phenyl]-1-[(4-methyl-4′-{[(2-thienylmethyl)amino]carbonyl}-3-biphenylyl)methyl]-1H-indole-2-carboxylic acid

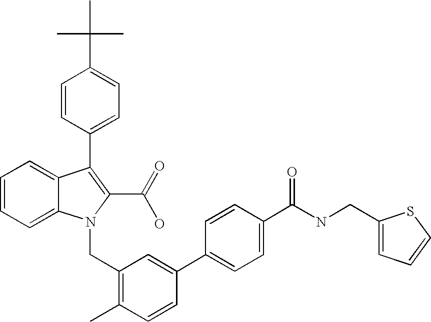

The title compound was obtained in 34% overall yield as a white foam from 3′-({2-{[(1,1-dimethylethyl)oxy]carbonyl}-3-[4-(1,1-dimethylethyl)phenyl]-1H-indol-1-yl}methyl)-4′-methyl-4-biphenylcarboxylic acid (Intermediate 15) and (2-thienylmethyl) as described for the synthesis of Example 30: 1H NMR (400 MHz, DMSO-d6), δ 9.07 (t, 1H, J=6.1 Hz), 7.79 (d, 2H, J=8.2 Hz), 7.5-7.29 (m, 12H), 7.11 (t, 1H, J=7.0 Hz), 6.97-6.91 (m, 2H), 6.32 (s, 1H), 5.85 (s, 2H), 4.58 (d, 2H, J=5.8 Hz), 2.42 (s, 3H), 1.33 (s, 9H); MS (ESI) m/z 627 (MH+)

### Example 33

### 3-[4-(1,1-dimethylethyl)phenyl]-1-{[4-methyl-4′-({[2-(2-thienyl)ethyl]amino}carbonyl)-3-biphenylyl]methyl}-1H-indole-2-carboxylic acid

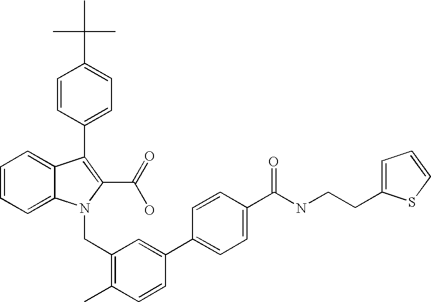

The title compound was obtained in 6% overall yield as a white foam from 3′-({2-{[(1,1-dimethylethyl)oxy]carbonyl}-3-[4-(1,1-dimethylethyl)phenyl]-1H-indol-1-yl}methyl)-4′-methyl-4-biphenylcarboxylic acid (Intermediate 15) and [2-(2-thienyl)ethyl]amine as described for the synthesis of Example 30: 1H NMR (400 MHz, CDCl₃). δ 7.64 (d, 1H, J=8.0 Hz), 7.58 (d, 2H, J=6.8 Hz), 7.48-7.42 (m, 4H), 7.37-7.22 (m, 7H), 7.16-7.12 (m, 1H), 6.94-6.91 (m, 1H), 6.84 (s, 1H), 6.54 (s, 1H), 6.22 (t, 1H, J=5.9 Hz), 5.81 (s, 2H), 3.68 (q, 2H, J=7.0 Hz), 3.10 (t, 2H, J=6.4 Hz), 2.44 (s, 3H), 1.38 (s, 9H); MS (ESI) m/z 613 (MH+)

### Example 34

### 3-[4-(1,1-dimethylethyl)phenyl]-1-({3-[4-(methylsulfonyl)-1-piperazinyl]phenyl}methyl)-1H-indole-2-carboxylic acid

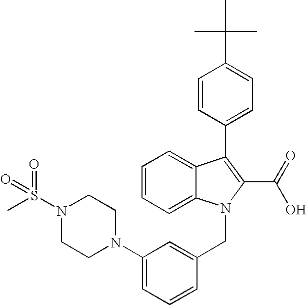

To 60 mg (0.12 mmol) of ethyl 3-(4-tert-butylphenyl)-1-(3-piperazin-1-ylbenzyl)-1H-indole-2-carboxylate (Intermediate 4) and 35 uL (0.25 mmol) TEA in 1.0 mL CH₂Cl₂ at 0° C. was added 12 uL (0.14 mmol) of MsCl and the solution stirred at room temperature for 12 hr. 25 mL CH₂Cl₂ was added and the solution washed with 20 mL sat. NaHCO₃ (aq) and 20 brine, dried over Na₂SO₄, concentrated and purified by silica gel chromatography (12 grams of silica gel eluting with 0-30% EtOAc in hexanes over 45 minutes.) The fractions containing product were concentrated, the residue taken up in 1 mL THF, 2 mL EtOH and 1 mL H₂O then 30 mg LiOH was added and the solution stirred at 50° C. overnight. The solution was concentrated to ⅓ volume then the pH was adjusted to 5.0 with 1.0 N HCl (aq.) The resulting solids were collected by suction filtration, washed with H₂O and dried to yield 28 mg (42%) of the title compound as a white solid: 1H NMR (400 MHz, DMSO-d6), δ 7.49 (d, 2H, J=8.1 Hz), 7.44-7.38 (m, 4H), 7.18 (t, 1H, J=7.4 Hz), 7.09-7.02 (m, 2H), 6.96 (s, 1H), 6.78 (d, 1H, J=8.2 Hz), 6.53 (d, 1H, J=6.8 Hz), 5.65 (s, 2H), 3.39-3.22 (m, 4H), 3.22-3.11 (m, 4H), 2.87 (s, 3H), 1.31 (s, 9H); MS (APCI) m/z 546 (MH+)

### Example 35

### 1-{[3-(4-acetyl-1-piperazinyl)phenyl]methyl}-3-[4-(1,1-dimethylethyl)phenyl]-1H-indole-2-carboxylic acid

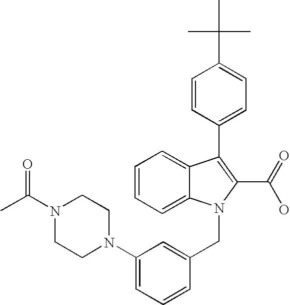

To 65 mg (0.12 mmol) of phenylmethyl 3-[4-(1,1-dimethylethyl)phenyl]-1-{[3-(1-piperazinyl)phenyl]methyl}-1H-indole-2-carboxylate (Intermediate 16) and 33 uL (0.23 mmol) TEA in 1.0 mL CH₂Cl₂ at 0° C. was added 10 uL (0.14 mmol) of acetyl chloride and the solution stirred at room temperature for 12 hr. 25 mL EtOAc was added and the solution washed with 20 mL H₂O and 20 mL brine, dried over Na₂SO₄, concentrated and purified by silica gel chromatography (12 grams of silica gel eluting with 0-20% EtOAc in hexanes over 45 minutes.) The fractions containing product were concentrated, the residue taken up in 4 mL CHCl₃ and 1 mL MeOH, 20 mg Pd/C (10% Degussa type) was added and the mixture stirred under 1 atm H₂ for 12 hr. The mixture was filtered through a pad of Celite then concentrated to yield 26 mg (44%) of the title compound as a light purplish foam: 1H NMR (400 MHz, DMSO-d6), δ 7.59 (d, 1H, J=8.4 Hz), 7.50-7.42 (m, 3H), 7.39-7.22 (m, 3H), 7.16-7.08 (m, 2H), 6.90-6.83 (m, 2H), 6.44 (d, 1H, J=7.5 Hz), 5.75 (s, 2H), 3.58-3.49 (m, 4H), 3.18-3.05 (m, 4H), 2.02 (s, 3H), 1.32 (s, 9H); MS (ESI) m/z 510 (MH+)

### Example 36

### 3-[4-(1,1-dimethylethyl)phenyl]-1-[(3-{4-[(methyloxy)carbonyl]-1-piperazinyl}phenyl)methyl]-1H-indole-2-carboxylic acid

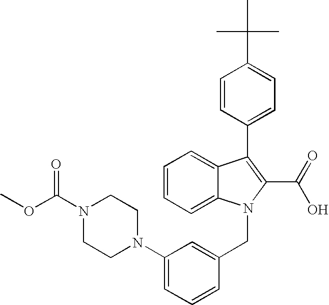

The title compound was obtained in 69% overall yield as a light-purplish foam from phenylmethyl 3-[4-(1,1-dimethylethyl)phenyl]-1-{[3-(1-piperazinyl)phenyl]methyl}-1H-indole-2-carboxylate (Intermediate 16) and methyl chloridocarbonate as described for the synthesis of Example 35: 1H NMR (400 MHz, DMSO-d6) δ 7.59 (d, 1H, J=8.4 Hz), 7.52-7.30 (m, 6H), 7.18-7.11 (m, 2H), 6.95-6.85 (m, 2H), 6.43 (d, 1H, J=7.5 Hz), 5.74 (s, 2H), 3.59 (s, 3H), 3.51-3.42 (m, 4H), 3.14-3.03 (m, 4H), 1.32 (s, 9H); MS (ESI) m/z 526 (MH+)

### Example 37

### 1-({3-[4-(aminocarbonyl)-1-piperazinyl]phenyl}methyl)-3-[4-(1,1-dimethylethyl)phenyl]-1H-indole-2-carboxylic acid

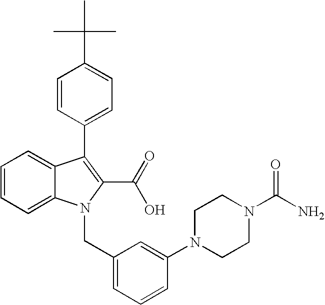

To 77 mg (0.16 mmol) of ethyl 3-(4-tert-butylphenyl)-1-(3-piperazin-1-ylbenzyl)-1H-indole-2-carboxylate (intermediate 4) in 0.5 mL AcOH and 0.5 mL H₂O was added 22 mg (0.27 mmol) KNCO and the mixture stirred at 40° C. for 10 min. An additional 0.15 mmol of KNCO was added and the mixture stirred for an additional 1 hr. 20 mL EtOAc was added and the solution washed with with 20 mL sat. NaHCO₃ (aq) and 20 mL brine then dried over Na₂SO₄, concentrated and purified by silica gel chromatography (12 grams of silica gel eluting with 0-60% acetone in CH₂Cl₂ over 45 minutes.) The fractions containing product were concentrated, the residue taken up in 1.0 mL THF, 2.0 mL EtOH and 1.0 mL H₂O and 40 mg (0.82 mmol) NaOH was added. After stirring at room temperature overnight, the solution was concentrated to ⅓ volume then made acid to litmus with 1.0 N HCl (aq). The resulting solids were collected by suction filtration, washed with H₂O and dried to yield 38 mg (48%) of the title compound as a white solid: 1H NMR (400 MHz, DMSO-d6), δ 12.96 (bs, 1H), 7.60 (s, 1H, J=8.4 Hz), 7.51-7.25 (m, 6H), 7.16-7.05 (m, 2H), 6.92-6.85 (m, 2H), 6.40 (d, 1H, J=7.5 Hz), 6.02 (s, 2H), 5.74 (s, 2H), 3.43-2.25 (m, 4H), 3.06-2.95 (m, 4H), 1.35 (s, 9H); MS (ESI) m/z 509 (MH−)

### Example 38

### 1-[(3-{4-[({[(1,1-dimethylethyl)oxy]carbonyl}amino)sulfonyl]-1-piperazinyl}phenyl)methyl]-3-[4-(1,1-dimethylethyl)phenyl]-1H-indole-2-carboxylic acid

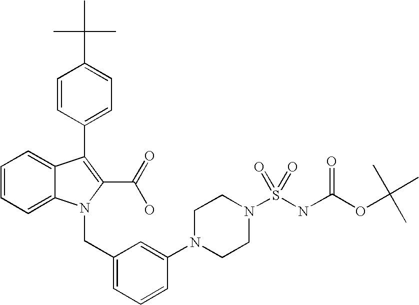

A solution of 35 uL (0.39 mmol) of isocyanatidosulfuryl chloride in 0.5 mL CH₂Cl₂ was added to a solution of 62 uL (0.65 mmol) of t-BuOH in 0.5 mL CH₂Cl₂ at 0° C. The solution was warmed to room temperature then was added to a solution of 60 uL (0.43 mmol) TEA and 200 mg (0.36 mmol) of phenylmethyl 3-[4-(1,1-dimethylethyl)phenyl]-1-{[3-(1-piperazinyl)phenyl]methyl}-1H-indole-2-carboxylate (intermediate 16) in 2 mL CH₂Cl₂ then stirred at room temperature for 1 hr. The solvent was evaporated and the residue taken up in 25 mL EtOAc, washed with 25 mL H₂O and 25 mL brine then dried over Na2SO4, concentrated and purified by silica gel chromatography (12 grams of silica gel eluting with 0-25% EtOAc in hexanes over 45 minutes.) The fractions containing product were concentrated, the residue taken up 4 mL CHCl₃ and 1 mL MeOH and 10 mg Pd/C (10% Degussa type) was added. The mixture was stirred under 1 atm of H₂ for 12 hr then filtered through a pad of Celite. The filtrate was concentrated to dryness and the residue taken up in a minimum of EtOAc the triturated with hexanes. The resulting solids were collected by suction filtration and dried to yield 21 mg (27%) of the title compound as a white solid: 1H NMR (400 MHz, DMSO-d6), δ 11.06 (s, 1H), 7.58 (d, 1H, J=8.4 Hz), 7.49-7.24 (m, 6H), 7.15-7.04 (m, 2H), 6.88 (s, 1H), 6.80 (d, 1H, J=8.2 Hz), 6.39 (d, 1H, J=7.5 Hz), 5.74 (s, 2H), 3.38-3.25 (m, 4H), 3.19-3.09 (m, 4H), 1.41 (s, 9H), 1.32 (s, 9H); MS (ESI) m/z 647 (MH+)

### Example 39

### 1-({3-[4-(aminosulfonyl)-1-piperazinyl]phenyl}methyl)-3-[4-(1,1-dimethylethyl)phenyl]-1H-indole-2-carboxylic acid

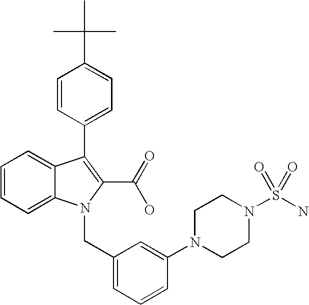

A solution of 120 mg (0.16 mmol) of phenylmethyl 1-[(3-{4-[({[(1,1-dimethylethyl)oxy]carbonyl}amino)sulfonyl]-1-piperazinyl}phenyl)methyl]-3-[4-(1,1-dimethylethyl)phenyl]-1H-indole-2-carboxylate (see example 38) in 4 mL CH₂Cl₂ and 1 mL TFA was stirred at room temperature for 4 hr. The solution was evaporated and the residue taken up in 20 mL EtOAc then washed with 20 mL sat. Na₂CO₃ (aq), 20 mL H₂O and 20 mL brine. The solution was dried over Na₂SO₄, concentrated and purified by silica gel chromatography (12 grams of silica gel eluting with 0-60% EtOAc in hexanes over 45 minutes.) The fractions containing product were concentrated, the residue taken up 4 mL CHCl₃ and 1 mL MeOH and 10 mg Pd/C (10% Degussa type) was added. The mixture was stirred under 1 atm of H₂ for 12 hr then filtered through a pad of Celite. The filtrate was concentrated to dryness and the residue taken up in a minimum of EtOAc the triturated with hexanes. The resulting solids were collected by suction filtration and dried to yield 24 mg (26%) of the title compound as a light gray solid: 1H NMR (400 MHz, DMSO-d6) δ 7.60 (d, 1H, J=8.4 Hz), 7.45-7.24 (m, 4H), 7.18-7.11 (m, 1H), 6.95-6.82 (m, 3H), 6.41-6.37 (m, 1H), 5.75 (s, 2H), 3.22-3.15 (m, 4H), 3.15-3.02 (m, 4H), 1.32 (s, 9H); MS (APCI) m/z 547 (MH+)

### Example 40

### 1-[(3-[(cyclopropylmethyl)oxy]-5-{[2-(dimethylamino)ethyl]oxy}phenyl)methyl]-3-[4-(1,1-dimethylethyl)phenyl]-1H-indole-2-carboxylic acid

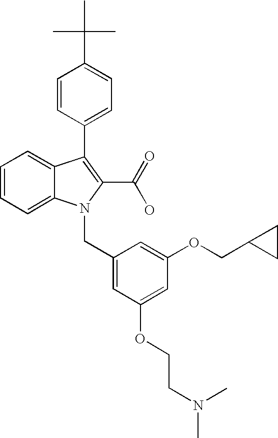

To solution of 75 mg (0.15 mmol) of ethyl 3-(4-tert-butylphenyl)-1-[3-(cyclopropylmethoxy)-5-hydroxybenzyl]-1H-indole-2-carboxylate (intermediate 1) in 1 mL DMF was added 43 mg (0.30 mmol) (2-chloroethyl)dimethylamine hydrochloride and 84 mg (0.60 mmol) K₂CO₃ and the mixture stirred at 80° C. for 12 hr. 25 mL EtOAc was added and the solution washed with three 25 mL portions of H₂O and 25 mL brine then dried over Na₂SO₄. The solution was concentrated and the residue taken up in 1 mL THF, 2 mL EtOH and 1 mL H₂O then 52 mg (1.28 mmol) NaOH was added and the solution stirred at 50° C. for 12 hr. The solution was concentrated to ⅓ volume then acidified to pH 5.0 with 1.0 N HCl (aq). The resulting solids were collected by suction filtration, washed with H₂O and dried to yield 39 mg (48%) of the title compound as a tan solid: 1H NMR (400 MHz, CDCl₃). δ 7.66 (d, 1H, J=7.9 Hz), 7.55-7.23 (m, 6H), 7.08 (t, 1H, J=7.3 Hz), 6.38 (s, 1H), 6.31 (s, 1H), 6.13 (s, 1H), 5.64 (s, 2H), 3.94 (s, 2H), 3.56 (d, 2H, J=7.0 Hz), 2.88-2.82 (m, 2H), 2.37 (s, 6H), 1.32 (s, 9H), 0.59-0.44 (m, 2H), 0.26-0.15 (m, 2H); MS (ESI) m/z 541 (MH+)

### Example 41

### 1-[(3-[(cyclopropylmethyl)oxy]-5-{[2-(1-pyrrolidinyl)ethyl]oxy}phenyl)methyl]-3-[4-(1,1-dimethylethyl)phenyl]-1H-indole-2-carboxylic acid

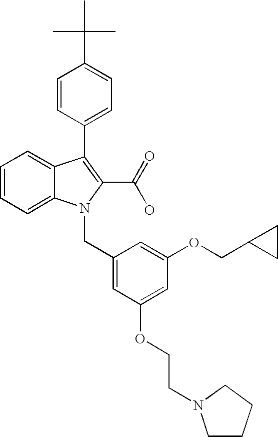

The title compound was obtained in 57% overall yield as a light-purplish foam from ethyl 3-(4-tert-butylphenyl)-1-[3-(cyclopropylmethoxy)-5-hydroxybenzyl]-1H-indole-2-carboxylate (intermediate 1) and 1-(2-chloroethyl)pyrrolidine hydrochloride as described for the synthesis of Example 40: 1H NMR (400 MHz, DMSO-d6) [symbol] 37.50 (d, 2H, J=8.2 Hz), 7.45-7.36 (m, 4H), 7.22 (t, 1H, J=7.3 Hz), 7.06 (t, 1H, J=7.5 Hz), 6.33 (s, 1H), 6.29 (s, 2H), 5.65 (s, 2H), 4.07 (t, 2H, J=5.2 Hz), 3.68 (d, 2H, J=7.0 Hz), 3.14-3.09 (m, 2H), 2.95-2.85 (m, 4H), 1.79-1.70 (m, 4H), 1.32 (s, 9H), 0.55-0.48 (m, 2H), 0.24-0.17 (m, 2H); MS (ESI) m/z 567 (MH+)

### Example 42

### 1-[(3-[(cyclopropylmethyl)oxy]-5-{[2-(4-morpholinyl)ethyl]oxy}phenyl)methyl]-3-[4-(1,1-dimethylethyl)phenyl]-1H-indole-2-carboxylic acid

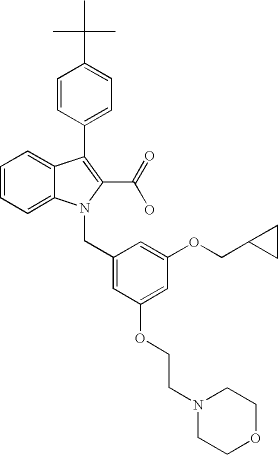

The title compound was obtained in 55% overall yield as a light-purplish foam from ethyl 3-(4-tert-butylphenyl)-1-[3-(cyclopropylmethoxy)-5-hydroxybenzyl]-1H-indole-2-carboxylate (intermediate 1) and 4-(2-chloroethyl)morpholine hydrochloride as described for the synthesis of Example 40: 1H NMR (400 MHz, DMSO-d6), δ 7.56 (d, 1H, J=8.2 Hz), 7.55-7.47 (m, 3H), 7.42-7.35 (m, 2H), 7.30 (t, 1H, J=7.5 Hz), 7.10 (t, 1H, 7.5 Hz), 6.36 (s, 1H), 6.21 (s, 2H), 5.72 (s, 2H), 4.18-4.10 (m, 2H), 3.78-3.69 (m, 4H), 3.05-2.66 (m, 6H), 1.32 (s, 9H), 0.56-0.47 (m, 2H), 0.29-0.20 (m, 2H); MS (ESI) m/z 583 (MH+)

### Example 43

### 1-[(3-[(cyclopropylmethyl)oxy]-5-{[3-(dimethylamino)propyl]oxy}phenyl)methyl]-3-[4-(1,1-dimethylethyl)phenyl]-1H-indole-2-carboxylic acid

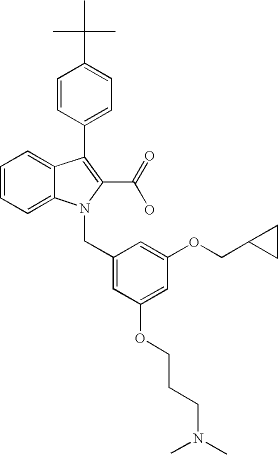

The title compound was obtained in 32% overall yield as a light-purplish foam from ethyl 3-(4-tert-butylphenyl)-1-[3-(cyclopropylmethoxy)-5-hydroxybenzyl]-1H-indole-2-carboxylate (intermediate 1) and (3-chloropropyl)dimethylamine hydrochloride as described for the synthesis of Example 40: 1H NMR (400 MHz, DMSO-d6) δ 7.54-7.50 (m, 2H), 7.43-7.39 (m, 4H), 7.22 (t, 1H, J=7.3 Hz), 7.06 (t, 1H, J=7.3 Hz), 6.35-6.29 (m, 3H), 5.65 (s, 2H), 3.88 (t, 2H, J=6.2 Hz), 3.67 (d, 2H, J=7.0 Hz), 2.85 (t, 2H, J=7.5 Hz), 2.50 (s, 3H), 2.47 (s, 3H), 1.98-1.90 (m, 2H), 1.30 (s, 9H), 1.18-1.10 (s, 1H), 0.58-0.45 (m, 2H), 0.23-0.15 (m, 2H); MS (ESI) m/z 555 (MH+)

### Example 44

### 3-[4-(1,1-dimethylethyl)phenyl]-1-{[3-(4-thiomorpholinyl)phenyl]methyl}-1H-indole-2-carboxylic acid

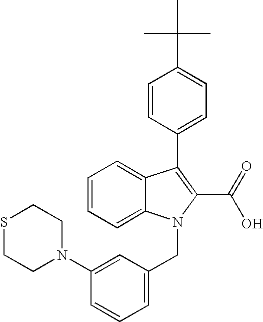

To 107 mg (0.21 mmol) of ethyl 3-(4-tert-butylphenyl)-1-(3-thiomorpholin-4-ylbenzyl)-1H-indole-2-carboxylate (Intermediate 5) in 2 mL EtOH, 1 mL THF and 1 mL H₂O was added 85 mg (2.10 mmol) NaOH and the solution stirred at 50° C. for 12 hr. The solution was concentrated to ⅓ volume then acidified with 1.0 N HCl (aq.) The resulting solids were collected by suction filtration, washed with H₂O and dried to yield 82 mg (81%) of the title compound as an off-white solid: 1H NMR (400 MHz, DMSO-d6) δ 7.60 (d, 1H, J=8.2 Hz), 7.55-7.27 (m, 6H), 7.19-7.05 (m, 2H), 6.99-6.79 (m, 2H), 6.45-6.38 (m, 1H), 5.75 (s, 2H), 3.55-3.39 (m, 4H), 2.75-2.59 (m, 4H), 1.32 (s, 9H); MS (APCI) m/z 484 (MH+)

### Example 45

### 3-[4-(1,1-dimethylethyl)phenyl]-1-{[3-(1,1-dioxido-4-thiomorpholinyl)phenyl]methyl}-1H-indole-2-carboxylic acid

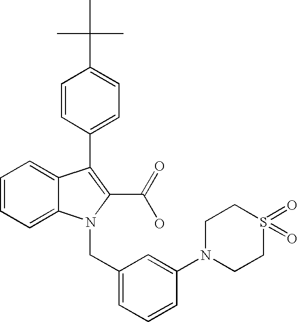

To 82 mg (0.16 mmol) of ethyl 3-(4-tert-butylphenyl)-1-(3-thiomorpholin-4-ylbenzyl)-1H-indole-2-carboxylate (Intermediate 5) in 2 mL acetone and 0.5 mL H₂O was added 57 mg (0.48 mmol) of N-NMO and 15 uL OsO₄ (2.5% in t-BuOH) and the solution stirred at room temperature for 12 hr. 25 mL EtOAc was added and the solution washed with 20 mL 10% Na₂S₂O₃ (aq), 20 mL H₂O, and 20 mL brine then dried over Na₂SO₄, filtered through a pad of Celite and concentrated. To this residue was added 2 mL EtOH, 1 mL THF and 1 mL H₂O followed by 65 mg (1.65 mmol) NaOH and the solution stirred at room temperature for 24 hr. The solution was concentrated to ⅓ volume and acidified with 1.0 N HCl (aq.) The resulting solids were collected by suction filtration and dried to yield 54 mg (65%) of the title compound as a white solid: 1H NMR (400 MHz, DMSO-d6) δ 7.51-7.38 (m, 6H), 7.19-6.99 (m, 4H), 6.85-6.80 (m, 1H), 6.62-6.58 (m, 1H), 5.61 (s, 2H), 3.79-3.65 (m, 4H), 3.08-2.98 (m, 4H), 1.31 (s, 9H); MS (APCI) m/z 561 (MH+)

### Example 46

### 3-[4-(1,1-dimethylethyl)phenyl]-1-[(3-{4-[(ethyloxy)carbonyl]-1-piperazinyl}phenyl)methyl]-1H-indole-2-carboxylic acid

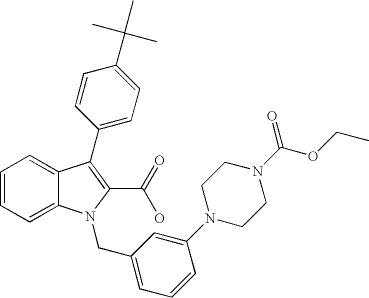

To a solution of 75 mg (0.13 mmol) of phenylmethyl 3-[4-(1,1-dimethylethyl)phenyl]-1-{[3-(1-piperazinyl)phenyl]methyl}-1H-indole-2-carboxylate (intermediate 16) and 38 uL (0.27 mmol) TEA in 1.5 mL CH₂Cl₂ at 0° C. was added 16 uL (0.16 mmol) ethyl chloridocarbonate and the solution stirred at room temperature for 12 hr. 25 mL EtOAc was added then the solution was washed with 20 mL H₂O and 20 mL brine then concentrated and the residue purified by silica gel chromatography (12 grams of silica gel eluting with 0-25% EtOAc in hexanes over 45 minutes.) The fractions containing product were concentrated, the residue taken up in 5 mL CHCl₃ and 1 mL MeOH and 10 mg Pd/C (10% Degussa type) was added. The mixture was stirred at room temperature under 1 atm H₂ for 1 hr. The solution was filtered through a pad of Celite, concentrated and the residue purified by silica gel chromatography (12 grams of silica gel eluting with 0-60% EtOAc in hexanes over 40 minutes) to yield 10 mg (14%) of the title compound as a gray foam: 1H NMR (400 MHz, CDCl₃) 6 7.59 (d, 1H, J=8.0 Hz), 7.48-7.27 (m, 6H), 7.18-7.10 (m, 2H), 6.77 (d, 1H, J=8.0 Hz), 6.71 (s, 1H), 6.58 (d, 1H, J=7.5 Hz), 5.79 (s, 2H), 4.11 (q, 2H, J=7.2 Hz), 3.60-3.52 (m, 4H), 3.07-2.99 (m, 4H), 1.38 (s, 9H), 1.25 (t, 3H, J=7.2 Hz); MS (ESI) m/z 540 (MH+)

### Example 47

### 3-[4-(1,1-dimethylethyl)phenyl]-1-{[3-(4-{[(1-methylethyl)oxy]carbonyl}-1-piperazinyl)phenyl]methyl}-1H-indole-2-carboxylic acid

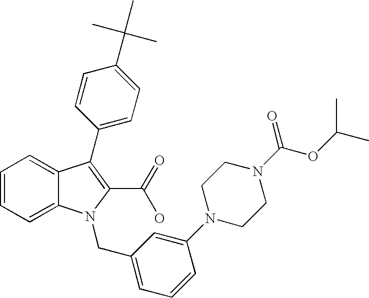

The title compound was obtained in 34% yield from phenylmethyl 3-[4-(1,1-dimethylethyl)phenyl]-1-{[3-(1-piperazinyl)phenyl]methyl}-1H-indole-2-carboxylate (intermediate 16) and 1-methylethyl chloridocarbonate as described for the synthesis of Example 46: 1H NMR (400 MHz, CDCl₃) δ 7.57 (d, 1H, J=7.9 Hz), 7.45-6.96 (m, 8H), 6.70-6.45 (m, 3H), 5.62 (s, 2H), 4.89 (t, 1H, J=6.0 Hz), 3.49-3.22 (m, 4H), 2.95-2.78 (m, 4H), 1.39-1.10 (m, 14H); MS (ESI) m/z 554 (MH+)

### Example 48

### 3-[4-(1,1-dimethylethyl)phenyl]-1-({3-[4-({[2-(methyloxy)ethyl]oxy}carbonyl)-1-piperazinyl]phenyl}methyl)-1H-indole-2-carboxylic acid

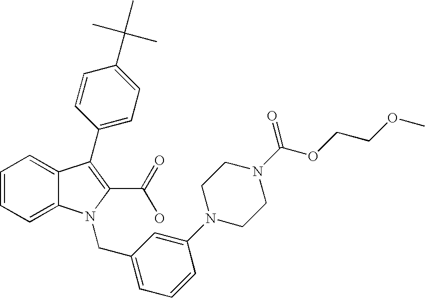

The title compound was obtained in 42% yield from phenylmethyl 3-[4-(1,1-dimethylethyl)phenyl]-1-{[3-(1-piperazinyl)phenyl]methyl}-1H-indole-2-carboxylate (intermediate 16) and 2-(methyloxy)ethyl chloridocarbonate as described for the synthesis of Example 46: 1H NMR (400 MHz, DMSO-d6) δ 7.58-7.41 (m, 6H), 7.21 (t, 1H, J=7.7 Hz), 7.07-7.01 (m, 2H), 6.89 (s, 1H), 6.78 (d, 1H, J=8.3 Hz), 6.47 (d, 1H, J=8.3 Hz), 5.72 (s, 2H), 4.11 (t, 2H, J=4.7 Hz), 3.55-3.44 (m, 6H), 3.24 (s, 3H), 3.07-2.99 (m, 4H), 1.39 (s, 9H); MS (ESI) m/z 570 (MH+)

### Example 49

### 1-[(3-{[(dimethylamino)carbonyl]oxy}-5-{[2-(methyloxy)ethyl]oxy}phenyl)methyl]-3-[4-(1,1-dimethylethyl)phenyl]-1H-indole-2-carboxylic acid

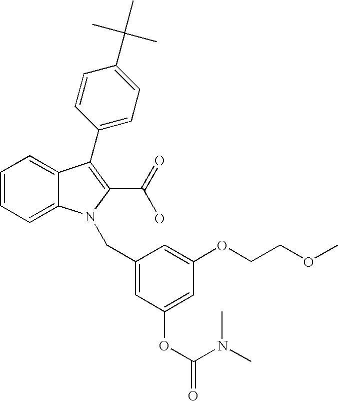

To a solution of 60 mg (0.11 mmol) of phenylmethyl 3-[4-(1,1-dimethylethyl)phenyl]-1-[(3-hydroxy-5-{[2-(methyloxy)ethyl]oxy}phenyl)methyl]-1H-indole-2-carboxylate (Intermediate 2), 25 uL (0.16 mmol) TEA and 5 mg of DMAP in 1 mL CH₂Cl₂ was added 12 uL (0.13 mmol) of dimethylcarbamic chloride and the mixture stirred at room temperature for 12 hr. 25 mL EtOAc was added and the solution washed with 20 mL H₂O and 20 mL brine then concentrated and the residue purified by silica gel chromatography (12 grams of silica gel eluting with 0-60% EtOAc in hexanes over 45 minutes.) The fractions containing product were concentrated, the residue taken up in 5 mL CHCl₃ and 1 mL MeOH and 10 mg Pd/C (10% Degussa type) was added. The mixture was stirred at room temperature under 1 atm H₂ for 1 hr. The mixture was filtered through a pad of Celite and concentrated to yield 42 mg (71%) of the title compound as a tan foam: 1H NMR (400 MHz, DMSO-d6) δ 12.98 (s, 1H), 7.57 (d, 1H, J=8.4 Hz), 7.55-7.45 (m, 3H), 7.37 (d, 2H, J=8.2 Hz), 7.30 (t, 1H, J=7.5 Hz), 7.11 (t, 1H, J=7.5 Hz), 6.57 (s, 1H), 6.44 (s, 2H), 5.75 (s, 2H), 3.96 (t, 2H, J=4.2 Hz), 3.55 (t, 2H, J=4.2 Hz), 3.22 (s, 3H), 2.95 (s, 3H), 2.84 (s, 3H), 1.32 (s, 9H); MS (ESI) m/z 567 (MH+)

### Example 50

### 3-[4-(1,1-dimethylethyl)phenyl]-1-[(3-{[2-(methyloxy)ethyl]oxy}-5-{[(4-methyl-1-piperazinyl)carbonyl]oxy}phenyl)methyl]-1H-indole-2-carboxylic acid

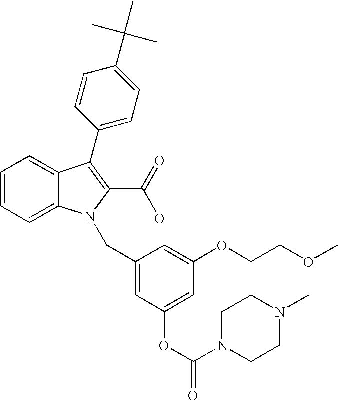

The title compound was obtained in 37% yield as a brown solid from phenylmethyl 3-[4-(1,1-dimethylethyl)phenyl]-1-[(3-hydroxy-5-{[2-(methyloxy)ethyl]oxy}phenyl)methyl]-1H-indole-2-carboxylate (Intermediate 2) and 4-methyl-1-piperazinecarbonyl chloride as described for the synthesis of Example 49: 1H NMR (400 MHz, DMSO-d6) δ 7.55 (d, 1H, J=8.4 Hz), 7.55-7.47 (m, 3H), 7.37 (d, 2H, J=8.4 Hz), 7.38-7.34 (m, 1H), 7.11 (t, 1H, J=7.7 Hz), 6.62 (s, 1H), 6.51 (s, 1H), 6.44 (s, 1H), 5.77 (s, 2H), 3.98 (t, 2H, J=4.0 Hz), 3.57 (t, 2H, J=4.7 Hz), 3.38-3.32 (m, 2H), 2.32 (s, 3H), 3.19-3.09 (m, 4H), 2.70-2.61 (m, 2H), 1.32 (s, 9H); MS (ESI) m/z 600 (MH+)

### Example 51

### 3-[4-(1,1-dimethylethyl)phenyl]-1-({3-{[2-(methyloxy)ethyl]oxy}-5-[(1-piperidinylcarbonyl)oxy]phenyl}methyl)-1H-indole-2-carboxylic acid

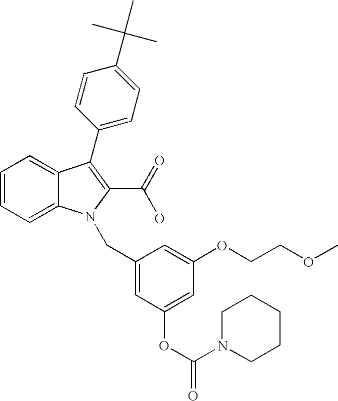

The title compound was obtained in 23% yield as a tan foam from phenylmethyl 3-[4-(1,1-dimethylethyl)phenyl]-1-[(3-hydroxy-5-{[2-(methyloxy)ethyl]oxy}phenyl)methyl]-1H-indole-2-carboxylate (Intermediate 2) and 1-piperidinecarbonyl chloride as described for the synthesis of Example 49: 1H NMR (400 MHz, CDCl₃) δ 7.59 (d, 1H, J=7.9 Hz), 7.51-7.40 (m, 4H), 7.38-7.30 (m, 2H), 7.19-7.12 (m, 1H), 6.55 (s, 2H), 6.45 (s, 1H), 5.75 (s, 2H), 3.97 (t, 2H, J=4.5 Hz), 3.63 (t, 2H, J=4.0 Hz), 3.59-3.41 (m, 6H), 3.34 (s, 3H), 1.68-1.55 (m, 6H), 1.38 (s, 9H)

### Example 52

### 3-[4-(1,1-dimethylethyl)phenyl]-1-({3-{[2-(methyloxy)ethyl]oxy}-5-[(4-morpholinylcarbonyl)oxy]phenyl}methyl)-1H-indole-2-carboxylic acid

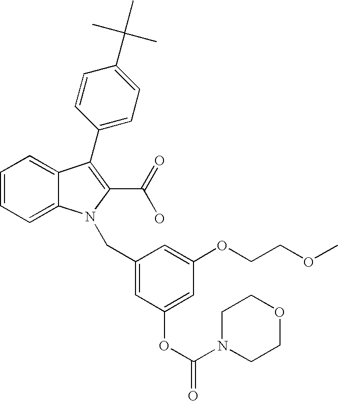

The title compound was obtained in 36% yield as an off-white foam from phenylmethyl 3-[4-(1,1-dimethylethyl)phenyl]-1-[(3-hydroxy-5-{[2-(methyloxy)ethyl]oxy}phenyl)methyl]-1H-indole-2-carboxylate (Intermediate 2) and 4-morpholinecarbonyl chloride as described for the synthesis of Example 49: 1H NMR (400 MHz, CDCl₃). δ 7.60 (d, 1H, J=8.0 Hz), 7.55-=7.49(m, 4H), 7.39-7.36 (m, 2H), 7.19-7.14 (m, 1H), 6.56 (s, 1H), 6.52 (s, 1H), 6.49 (s, 1H), 5.76 (s, 2H), 3.98 (t, 2H, J=4.4 Hz), 3.75-3.50 (m, 10H), 3.36 (s, 3H), 1.37 (s, 9H); MS (APCI) m/z 587 (MH+)

### Example 53

### 3-[4-(1,1-dimethylethyl)phenyl]-1-[(3-{[2-(methyloxy)ethyl]oxy}-5-{[(2-oxo-1-imidazolidinyl)carbonyl]oxy}phenyl)methyl]-1H-indole-2-carboxylic acid

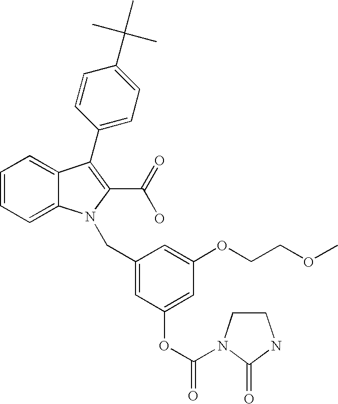

The title compound was obtained in 40% yield as a tan foam from phenylmethyl 3-[4-(1,1-dimethylethyl)phenyl]-1-[(3-hydroxy-5-{[2-(methyloxy)ethyl]oxy}phenyl)methyl]-1H-indole-2-carboxylate (Intermediate 2) using 2-oxo-1-imidazolidinecarbonyl chloride as described for the synthesis of Example 49: 1H NMR (400 MHz, DMSO-d6), δ 12.99 (s, 1H), 7.57 (d, 1H, J=8.5 Hz), 7.52 (s, 1H), 7.50-7.32 (m, 6H), 7.11 (t, 1H, J=7.5 Hz), 6.67 (s, 1H), 6.51 (s, 1H), 6.48 (s, 1H), 5.77 (s, 2H), 3.98 (t, 2H, J=4.0 Hz), 3.84 (t, 2H, J=4.3 Hz), 3.56 (t, 2H, J=4.3 Hz), 3.30-3.27 (m, 2H), 3.23 (s, 3H), 1.32 (s, 9H); MS (APCI) m/z 608 (M+Na)

### Example 54

### 1-[(3-[(cyclopropylmethyl)oxy]-5-{[2-(1H-pyrrol-1-yl)ethyl]oxy}phenyl)methyl]-3-{[3-(trifluoromethyl)phenyl]methyl}-1H-indole-2-carboxylic acid

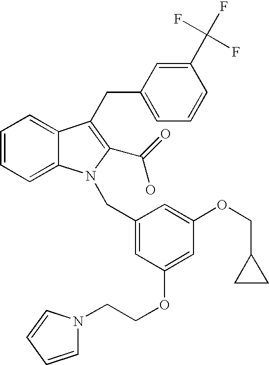

A mixture of intermediate 27 (150 mg, 0.29 mmol), 2-bromoethylpyrrole (75 mg, 0.43 mmol) and K₂CO₃ (79 mg, 0.57 mmol) in DMF (4 mL) was stirred at 60^{˜} C for 32 hours. The mixture was poured into water (50 mL) and extracted with ether (2×60 mL). The combined ether was washed with brine and concentrated. The crude ethyl ester was taken up in EtOH (6 mL), added a solution of KOH (2 mL, 20% in water) and stirred at 50^{˜} C for 2 hours. Poured into ice water (50 mL), added concentrated HCl to pH˜4 and extracted with ether (2×60 mL). The combined ether was washed with brine, dried over MgSO₄ and concentrated. Added 20% ether in hexane (8 mL) and stirred for 2 hours. The resulting solid was filtered, rinsed with hexane and dried under vacuum at 70^{˜} C to afford the title compound (91 mg, 54%) as a tan solid. ¹H NMR (400 MHz, DMSO-d₆); δ 13.32 (br, 1H), 7.69 (d, 1H), 7.60 (s, 1H), 7.50-7.40 (m,4H), 7.26 (t, 1H), 7.08 (t, 1H), 6.73 (t, 2H), 6.27 (s, 1H), 6.14 (s, 1H), 6.00 (s, 1H), 5.94 (t, 2H), 5.73 (s, 2H), 4.55 (s, 2H), 4.15 (t, 2H), 4.07 (t, 2H), 3.62 (d, 2H), 1.08-1.05 (m, 1H), 0.48-0.44 (m, 2H), 0.21-0.17 (m, 2H); MS m/z 589 (M+H); C₃₄H₃₁F₃N₂O₄. Calculated: C, 69.37; H, 5.31; N, 4.76; Found: C, 69.18; H, 5.27; N, 4.71.

### Example 55

### 1-({3-[(cyclopropylmethyl)oxy]-5-[(3-{[2-(methyloxy)ethyl]oxy}propyl)oxy]phenyl}methyl)-3-{[3-(trifluoromethyl)phenyl]methyl}-1H-indole-2-carboxylic acid

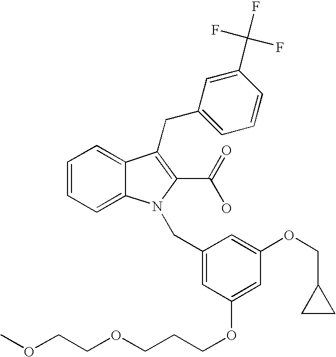

Prepared as previously described in example 54 using intermediate 27 (150 mg, 0.29 mmol) and 3-(methoxyethoxy)propylbromide (150 mg, 0.29 mmol) to afford the title compound (122 mg, 85%) as a white solid. ¹H NMR (400 MHz, DMSO-d₆); δ 13.25 (br, 1H), 7.69 (d, 1H), 7.59 (s, 1H), 7.52-7.43 (m, 4H), 7.26 (t, 1H), 7.08 (t, 1H), 6.27 (s, 1H), 6.07 (s, 1H), 6.04 (s, 1H), 5.74 (s, 2H), 4.55 (s, 2H), 3.85 (t, 2H), 3.63 (d, 2H), 3.45-3.37 (m, 6H), 3.18 (s, 3H), 1.85-1.78 (m, 2H), 1.12-1.03 (m, 1H), 0.50-0.45 (m, 2H), 0.22-0.18 (m, 2H); MS m/z 633 (M+Na); C₃₄H₃₆F₃N₁O₆. Calculated: C, 66.76; H, 5.93; N, 2.29; Found: C, 66.74; H, 5.88; N, 2.33.

### Example 56

### 1-[(3-[(cyclopropylmethyl)oxy]-5-{[2-(methyloxy)ethyl]oxy}phenyl)methyl]-3-{[3-(trifluoromethyl)phenyl]methyl}-1H-indole-2-carboxylic acid

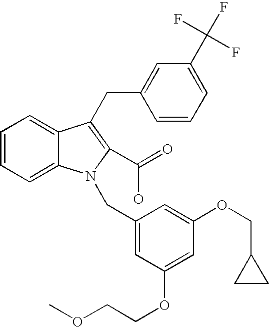

A mixture of intermediate 26 (300 mg, 0.86 mmol), intermediate 28 (300 mg, 0.95 mmol), K₂CO₃ 239 mg, 1.70 mmol) and DMF (4 mL) was stirred at ambient temperature for 72 hours. The mixture was poured into water (50 mL) and extracted with ether (2×50 mL). The combined ether was washed with brine (2×40 mL), dried over MgSO₄ and concentrated. The crude ethyl ester was taken up in EtOH (12 mL), added a solution of KOH (4 mL, 20% in water) and stirred at 50^{˜} C for 2 hours. The reaction was poured into water (60 mL) and extracted with ether (50 mL, discarded). The aqueous was acidified with 1N HCl and extracted with ether (2×60 mL). The combined ether was washed with brine, dried over MgSO₄ and concentrated to ˜10 mL. The resulting solid was filtered, rinsed with hexane and dried under vacuum at 70^{˜} C to afford the title compound (223 mg, 49%) as a white solid. ¹H NMR (400 MHz, DMSO-d₆); δ 13.33 (br, 1H), 7.69 (d, 1H), 7.60 (s, 1H), 7.50-7.42 (m, 4H), 7.27 (t, 1H), 7.08 (t, 1H), 6.29 (s, 1H), 6.07 (s, 1H), 6.04 (s, 1H), 5.74 (s, 2H), 4.55 (s, 2H), 3.91 (t, 2H), 3.64 (d, 2H), 3.53 (t, 2H), 3.22 (s, 3H), 1.12-1.06 (m, 1H), 0.50-0.46 (m, 2H), 0.22-0.19 (m, 2H); MS m/z 554(M+H); C₃₁H₃₀F₃N₁O₅. Calculated: C, 67.27; H, 5.47; N, 2.53; Found: C, 67.26; H, 5.46; N, 2.53.

### Example 57

### 1-[(3-[(cyclopropylmethyl)oxy]-5-{[3-(dimethylamino)propyl]oxy}phenyl)methyl]-3-{[3-(trifluoromethyl)phenyl]methyl}-1H-indole-2-carboxylic acid hydrochloride

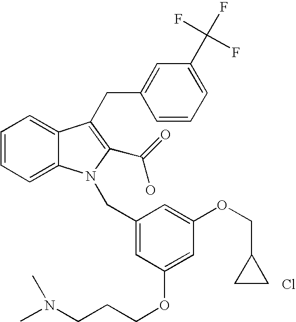

Prepared similarly as described in example 53 using intermediate 27 (200 mg, 0.38 mmol) and N,N-dimethylpropylchloride hydrochloride (91 mg, 0.57 mmol) to afford the title compound (201 mg, 85%) as a white solid. ¹H NMR (400 MHz, DMSO-d₆); δ 13.33 (br, 1H), 7.69 (d, 1H), 7.61 (s, 1H), 7.53-7.44 (m, 4H), 7.25 (t, 1H), 7.06 (t, 1H), 6.30 (s, 1H), 6.13 (s, 1H), 6.09 (s, 1H), 5.74 (s, 2H), 4.54 (s, 2H), 3.90 (t, 2H), 3.64 (d, 2H), 3.04 (t, 2H), 2.66 (s, 6H), 2.04-2.00 (m, 2H), 1.12-1.04 (m, 1H), 0.50-0.45 (m, 2H), 0.22-0.19 (m, 2H); High resolution MS m/z 581 (M+H); C₃₃H₃₅F₃N₂O₄.

### Example 58

### 1-[(3,5-bis{[2-(methyloxy)ethyl]oxy}phenyl)methyl]-3-{[3-(trifluoromethyl)phenyl]methyl}-1H-indole-2-carboxylic acid

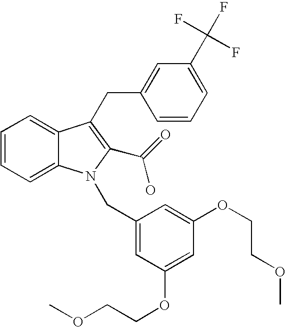

Prepared as previously described in example 56 using intermediate 26 (250 mg, 0.72 mmol) and intermediate 29 (237 mg, 0.86 mmol) to afford the title compound (230 mg, 57%) as an off-white solid. ¹H NMR (400 MHz, DMSO-d₆); δ 13.28 (br, 1H), 7.69 (d, 1H), 7.60 (s, 1H), 7.50-7.41 (m, 4H), 7.27 (t, 1H), 7.08 (t, 1H), 6.32 (t, 1H), 6.07 (d, 2H), 5.75 (s, 2H), 4.55 (s, 2H), 3.93-3.91 (m, 4H), 3.55-3.51 (m, 4H), 3.22 (s, 6H); MS m/z 558 (M+H); C₃₀H₃₀F₃N₁O₆. Calculated: C,64.62; H, 5.42; N, 2.51; Found: C, 64.61; H,5.46; N, 2.54.

### Example 59

### 1-({3,5-bis[(cyclopropylmethyl)oxy]phenyl}methyl)-3-{[3-(trifluoromethyl)phenyl]methyl}-1H-indole-2-carboxylic acid

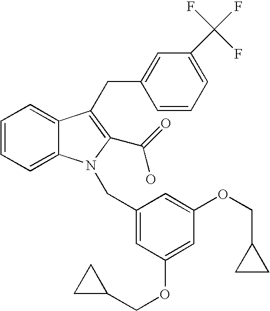

Prepared as previously described in Example 53 using Intermediate 27 (150 mg, 0.29 mmol) and cyclopropylmethylbromide (77 mg, 0.57 mmol) to afford the title compound (87 mg, 55%) as a white solid. ¹H NMR (400 MHz, DMSO-d₆); δ 13.25 (br, 1H), 7.69 (d, 1H), 7.59 (s, 1H), 7.52-7.42 (m, 4H), 7.27 (t, 1H), 7.08 (t, 1H), 6.25 (t, 1H), 6.04 (d, 2H), 5.73 (s, 2H), 4.55 (s, 2H), 3.64 (s, 2H), 3.62 (s, 2H), 1.13-1.03 (m, 2H), 0.50-0.42 (m, 4H), 0.25-0.18 (m, 4H); MS m/z 550 (M+H); C₃₂H₃₀F₃N₁O₄. Calculated: C, 69.93; H, 5.50; N, 2.55; Found: C, 69.94; H, 5.59; N, 2.38.

### Example 60

### 3-(1-benzofuran-2-yl)-1-[(3,5-bis{[2-(methyloxy)ethyl]oxy}phenyl)methyl]-1H-indole-2-carboxylic acid

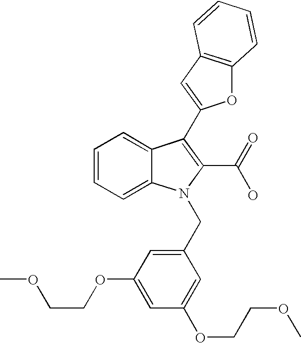

A solution of 0.50 g of Intermediate 30 and 0.285 ml of benzofuran in 15 ml of DCE was treated with 0.070 g of (Rh(OAc)₂)₂. The mixture under nitrogen was heated to 80° C. for 2 Hr. The reaction was allowed to cool to room temperature over night, filtered through silica gel and Celite, and concentrated in vacuo to give 0.67 g of crude product. Purification by chromatography [ISCO; RediSep; 40 g silica gel; eluting with 20-60% CH₂Cl₂/hexane] afforded 0.164 g of pure ethyl 3-(1-benzofuran-2-yl)-1H-indole-2-carboxylate. HPLC [Waters X-terra C-18; 30-100% CH₃CN/H₂O (0.1% TFA)/3 min; UV det.] RT=4.13 (100%). 1H NMR (DMSO-d6) δ 12.26 (s, 1H), 8.17 (d, 1H, J=8 Hz), 7.67 (m, 1H), 7.54 (m, 1H), 7.29 (m, 4H), 4.38 (q, 2H, J=7 Hz), 1.33 (t, 3H, J=7 Hz). MS ES+/−m/z 306 [M+H]+, 328 [M+Na]+, 304 [M−H]−.

A solution of 50 mg of ethyl 3-(1-benzofuran-2-yl)-1H-indole-2-carboxylate in 2 ml of anhydrous DMF was treated with >110 mg of Cs₂CO₃ followed by 49 mg of intermediate 29. The mixture was capped under nitrogen and heated at 60° C. over night. The reaction was diluted with EtOAc and water and partitioned. The organic layer was washed with sat. NaHCO₃ and brine and dried with Na₂SO₄ then concentrated in vacuo to give crude product which was purified by chromatography [ISCO; RediSep; 4 g silica gel; eluting with 5-40% EtOAc/hexane] to give 54 mg of ethyl 1-[3,5-bis-{(2-methoxyethoxy)}benzyl]-3-(1-benzofuran-2-yl)-1H-indole-2-carboxylate. HPLC [Waters X-terra C-18; 30-100% CH₃CN/H₂O (0.1% TFA)/3 min; UV det.] RT=4.62 (97%). MS ES+/−m/z 544 [M+H]+, 566 [M+Na]+.

A solution of 53 mg of ethyl 1-[3,5-bis-{(2-methoxyethoxy)}benzyl]-3-(1-benzofuran-2-yl)-1H-indole-2-carboxylate in 2 ml of MeOH was treated with 1.00 ml of 1.00 M NaOH. The mixture was heated at 60° C. for 8 hr then neutralized by addition of 1.00 ml of 1.00 M HCl and partially concentrated in vacuo. The remaining aqueous mixture was extracted twice with EtOAc. The EtOAc extracts were combined, dried with Na₂SO₄, and concentrated in vacuo to give 51 mg of the title compound (Example 60) as a pale yellow amorphous solid. HPLC [Waters X-terra C-18; 30-100% CH₃CN/H₂0 (0.1% TFA)/3 min; UV det.] RT=3.94 (98%). 1H NMR (DMSO-d6) δ 13.74 (bs, 1H), 7.98 (d, 1H, J=8 Hz), 7.64 (m, 1H), 7.31 (m, 5H), 6.38 (s, 1H), 6.22 (s, 2H), 5.71 (s, 2H), 3.97 (s, 4H), 3.56 (s, 4H), 3.23 (s, 6H). MS ES+/−m/z 516 [M+H]+, 538 [M+Na]+, 514 [M−H]−.

### Example 61

### 1-[(3,5-bis{[2-(methyloxy)ethyl]oxy}phenyl)methyl]-3-{[4-(1,1-dimethylethyl)phenyl]oxy}-1H-indole-2-carboxylic acid

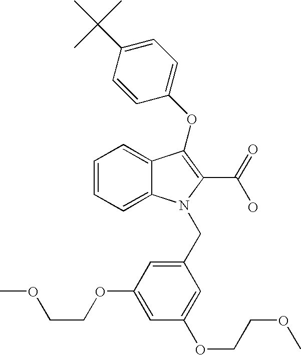

A solution of 0.525 g of Intermediate 30 and 0.425 g of 4-tert-butylphenol in 15 ml of DCE was treated with 0.118 g of (Rh(OAc)₂)₂. The mixture under nitrogen was heated at 80° C. for 2 hr. The reaction was allowed to cool to room temperature and stir over night then filtered through silica gel and Celite. The filtrate was concentrated in vacuo then purified by chromatography [ISCO; RediSep; 40 g silica gel; eluting with 5-30% EtOAc/hexane] to give 204 mg of ethyl 3-(4-tert-butylphenoxy)-1H-indole-2-carboxylate. HPLC [Waters X-terra C-18; 30-100% CH₃CN/H₂O (0.1% TFA)/3 min; UV det.] RT=4.30 (95%). 1H NMR (DMSO-d6) δ 11.70 (s, 1H), 7.44 (m, 1H), 7.28 (m, 4H), 7.01 (m, 1H), 6.81 (m, 2H), 4.17 (q, 2H, J=7 Hz), 1.22 (s, 9H), 1.07 (t, 3H, J=7 Hz). MS ES+/−m/z 360 [M+Na]+, 336 [M−H]−.

A solution of 50 mg of ethyl 3-(4-tert-butylphenoxy)-1H-indole-2-carboxylate in 2 ml of anhydrous DMF was treated with 0.10 g of Cs₂CO₃ followed by 45 mg of Intermediate 29. The mixture was capped under nitrogen and heated at 60° C. over night. The reaction was diluted with EtOAc and water and partitioned. The organic layer was washed with sat'd NaHCO₃ and brine and dried with Na₂SO₄ then concentrated in vacuo to give crude product which was purified by chromatography [ISCO; RediSep; 4 g silica gel; eluting with 5-50% EtOAc/hexane] to give 52 mg of ethyl 1-[3,5-bis-{(2-methoxyethoxy)}benzyl]-3-(4-tert-butyl phenoxy)-1H-indole-2-carboxylate. HPLC [Waters X-terra C-18; 30-100% CH₃CN/H₂O (0.1% TFA)/3 min; UV det.] RT=4.92 min (95%). MS ES+/−m/z 576 [M+H]+, 598 [M+Na]+.

A solution of 51 mg of ethyl 1-[3,5-bis-{(2-methoxyethoxy)}benzyl]-3-(4-tert-butylphenoxy)-1H-indole-2-carboxylate in 2 ml of methanol was treated with 1.00 ml of 1.00 M NaOH. The mixture was heated at 60° C. for 8 Hrs then neutralized by addition of 1.00 ml of 1.00 M HCl. The solution was partially concentrated in vacuo then extracted twice with EtOAc. The combined extracts were dried with Na₂SO₄ and concentrated in vacuo to give 45 mg of the title compound (Example 61) as an amorphous solid. 1H NMR (DMSO-d6) δ 13.01 (s, 1H), 7.06 (d, 1H, J=8 Hz), 7.29 (m, 4H), 7.05 (m, 1H), 6.80 (m, 2H), 6.35 (t, 1H, J=2 Hz), 6.13 (d, 2H, J=2 Hz), 5.77 (s, 2H), 3.96 (m, 4H), 3.57 (m, 4H), 3.24 (s, 6H), 1.23 (s, 9H). HPLC [Waters X-terra C-18; 30-100% CH₃CN/H₂O (0.1% TFA)/3 min; UV det.] RT=4.39 min (95%). MS ES+/−m/z 548 [M+H]+, 570 [M+Na]+, 546 [M−H]−.

### Example 62

### 1-[(3,5-bis{[2-(methyloxy)ethyl]oxy}phenyl)methyl]-3-{[4-(1,1-dimethylethyl)phenyl]amino}-1H-indole-2-carboxylic acid

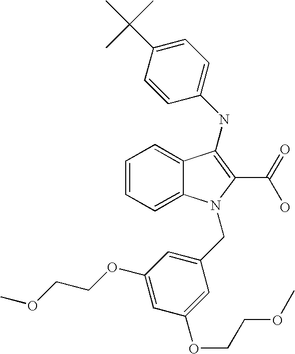

A solution of 402 mg of Intermediate 30 and 0.325 ml of 4-tertbutylaniline in 10 ml of DCE was treated with 81 mg of (Rh(OAc)₂)₂. The mixture was heated at 80° C. for 2 Hrs. The mixture was filtered through silica gel and Celite and concentrated in vacuo to give crude product which was purified by column chromatography [ISCO; RediSep; 40 g silica gel; eluting with 5-20% EtOAc/hexane] to give 360 mg of ethyl 3-[(4-tert-butylphenyl)amino]-1H-indole-2-carboxylate as a crystalline solid. HPLC [Waters X-terra C-18; 20-100% CH₃CN/H₂O (0.1% TFA)/3 min; UV det.] RT=4.64 min (99%). 1H NMR (DMSO-d6) δ 11.29 (s, 1H), 7.56 (s, 1H), 7.38 (d, 1H, J=8 Hz), 7.27 (d, 1H, J=8 Hz), 7.23 (m, 1H), 7.16 (d, 2H, J=9 Hz), 6.93 (m, 1H), 6.78 (d, 2H, J=9 Hz), 4.26 (q, 2H, J=7 Hz), 1.24 (t, 3H, J=7 Hz), 1.22 (s, 9H). MS ES+/−m/z 335 [M−H]−, 381 [M+formate]−.

Under nitrogen atmosphere and anhydrous conditions, a solution of 51 mg of ethyl 3-[(4-tert-butylphenyl)amino]-1H-indole-2-carboxylate in 2 ml of DMF was cooled to 0° C. and treated with 0.152 ml of NaHMDS as a 1.0 M solution in THF. The reaction was maintained at 0° C. for ˜20 min. then treated with 0.042 g of Intermediate 29 and allowed to come to room temperature as the ice bath melted over night. The reaction was diluted with 25 ml of water and extracted with 15 ml of EtOAc. The EtOAc extract was washed with 15 ml of aq. NaHCO₃ and 10 ml of brine then dried with Na₂SO₄ and concentrated in vacuo to give crude product. Purification by column chromatography [ISCO; RediSep; 4 g silica gel; eluting with 5-50% EtOAc/hexane] provided 70 mg of ethyl 1-[3,5-bis-{(2-methoxyethoxy)}benzyl]-3-[(4-tert-butylphenyl)amino]-1H-indole-2-carboxylate as a yellow resin. HPLC [Waters X-terra C-18; 30-100% CH₃CN/H₂O (0.1% TFA)/3 min; UV det.] RT=4.88 min (98%). MS ES+/−m/z 575 [M+H]+, 597 [M+Na]+.

A mixture of 69 mg of ethyl 1-[3,5-bis-{(2-methoxyethoxy)}benzyl]-3-[(4-tert-butylphenyl)amino]-1H-indole-2-carboxylate and 1.20 ml of 1.00 M NaOH in 2 ml of MeOH was heated at 65° C. over night. The reaction was neutralized by addition of 1.20 ml of 1.00 M HCl, and the resulting suspension was extracted with 15 ml of EtOAc which was dried with Na₂SO₄ and concentrated in vacuo to give 63 mg of the title compound (Example 62) as a yellow crystalline solid. HPLC [Waters X-terra C-18; 30-100% CH₃CN/H₂O (0.1% TFA)/3 min; UV det.] RT=4.20 min (86%). 1H NMR (DMSO-d6) δ 13.11 (bs, 1H), 7.74 (s, 1H), 7.52 (d, 1H, J=8 Hz), 7.27 (m, 1H), 7.17 (d, 2H, J=9 Hz), 6.98 (t, 1H, J=7.5 Hz), 6.79 (d, 2H, J=9 Hz), 6.33 (s, 1H), 6.10 (s, 2H), 5.70 (s, 2H), 3.95 (m, 4H), 3.56 (m, 4H), 3.23 (s, 6H), 1.22 (s, 9H). MS ES+/−m/z 547 [M+H]+, 569 [M+Na]+, 545 [M−H]−.

### Example 63

### 1-{[3,5-bis(trifluoromethyl)phenyl]methyl}-3-[4-(1,1-dimethylethyl)phenyl]-1H-indole-2-carboxylic acid

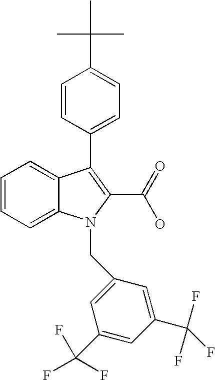

0.5 g (1.56 mM) of ethyl 3-[4-(1,1-dimethylethyl)phenyl]-1H-indole-2-carboxylate (WO 2002030895) was dissolved in 10 mL of DMF, 0.669 g (2.18 mM) of 1-(bromomethyl)-3,5-bis(trifluoromethyl)benzene and 1.02 g of Cs₂CO₃ were added and mixture was stirred for 16 hrs. The reaction mixture was diluted with water and product extracted with EtOAc. Organic layer was dried over MgSO₄ and solvent evaporated. Product was purified on SiO₂ with 35:65 mixture of EtOAc—hexane providing 0.46 g (54% yield). Product (300 mg) was dissolved in MeOH and 1N solution of NaOH was added. Mixture was stirred at 70° C. for 15 hrs. The MeOH was removed under reduced pressure and 1N HCl was added until pH=1 and product was extracted with EtOAc. Organic layer was dried over MgSO₄ and solvent evaporated providing 0.048 g (17% yield) of the title compound 1-{[3,5-bis(trifluoromethyl)phenyl]methyl}-3-[4-(1,1-dimethylethyl)phenyl]-1H-indole-2-carboxylic acid.

¹H NMR (400 MHz, Chloroform-d): δ 7.72 (b-s, 1H); 7.62 (b-d, 1H); 7.57 (b-s, 2H); 7.52-7.38 (m, 5H); 7.32 (b-d, 1H); 7.22-7.17 (m, 1H);5.90 (s, 2H); 1.39 (s, 9H) HPLC/MS ES [M−H]⁻=519.

### Example 64

### 3-[4-(1,1-dimethylethyl)phenyl]-1-{[3-{[2-(methyloxy)ethyl]oxy}-5-(trifluoromethyl)phenyl]methyl}-1H-indole-2-carboxylic acid

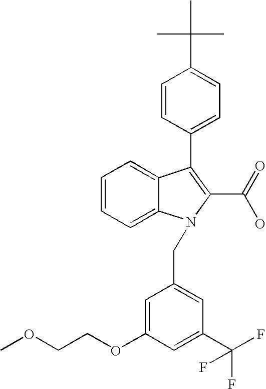

0.11 g (0.34 mM) of ethyl 344-(1,1-dimethylethyl)phenyl]-1H-indole-2-carboxylate (WO 2002030895) was dissolved in 5 mL of DMF, 0.123 g (0.48 mM) of 1-(chloromethyl)-3-{[2-(methyloxy)ethyl]oxy}-5-(trifluoromethyl)benzene (Intermediate 33) and 0.233 g of 05₂00₃ were added and mixture was stirred for 16 hrs. Reaction mixture was diluted with water and product extracted with EtOAc. The organic layer was dried over MgSO₄ and the solvent evaporated. The product was purified on SiO₂ with a 35:65 mixture of EtOAc—hexane providing 0.154 g (81% yield). Product was dissolved in MeOH and 1N solution of NaOH was added. The mixture was stirred at 70° C. for 15 hrs. The MeOH was removed under reduced pressure and 1N HCl was added until pH=1 and the product was extracted with EtOAc. The organic layer was dried over MgSO₄ and solvent evaporated providing 0.15 g (88% yield) of the title compound 344-(1,1-dimethylethyl)phenyl]-1-{[3-{[2-(methyloxy)ethyl]oxy}-5-(trifluoromethyl)phenyl]methyl}-1H-indole-2-carboxylic acid. ¹H NMR (400 MHz, DMSO-d₆): δ 7.62 (b-d, 2H); 7.51-7.28 (m, 6H); 7.16-7.07 (m, 3H); 6.81 (s, 1H); 5.86 (s, 2H); 4.08 (b-s, 2H); 3.57 (b-s, 2H); 1.32 (s, 9H) HPLC/MS ES [M−H]⁻=524.

### Example 65

### 1-{[3-[(cyclopropylmethyl)oxy]-5-(trifluoromethyl)phenyl]methyl}-3-[4-(1,1-dimethylethyl)phenyl]-1H-indole-2-carboxylic acid

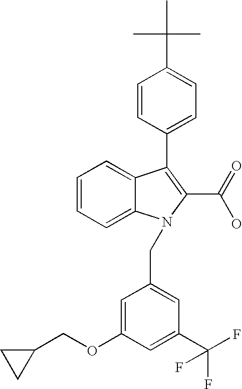

0.11 g (0.34 mM) of ethyl 3-[4-(1,1-dimethylethyl)phenyl]-1H-indole-2-carboxylate (WO 2002030895) was dissolved in 5 mL of DMF and 0.123 g (0.48 mM) of 1-(chloromethyl)-3-[(cyclopropylmethyl)oxy]-5-(trifluoromethyl)benzene (Intermediate 34) and 0.233 g of Cs₂CO₃ were added and mixture was stirred for 16 hrs. The reaction mixture was diluted with water and the product was extracted with EtOAc. The organic layer was dried over MgSO₄ and the solvent was evaporated. The product was purified on SiO₂ with 35:65 mixture of EtOAc—hexane elution providing 0.154 g (81% yield). To the product in methanol was added a 1N solution of NaOH. The mixture was stirred at 70° C. for 15 hrs. The MeOH was removed under reduced pressure and 1N HCl was added until the pH=˜1 and the product was extracted with EtOAc. The organic layer was dried over MgSO₄ and the solvent was evaporated providing 0.15 g (88% yield) of the title compound 1-{[3-[(cyclopropylmethyl)oxy]-5-(trifluoromethyl)phenyl]methyl}-3-[4-(1,1-dimethylethyl)phenyl]-1H-indole-2-carboxylic acid. ¹H NMR (400 MHz, DMSO-d₆): 7.62 (b-d, 1H); δ 7.50-7.44 (m, 3H); 7.40-7.30 (m, 3H); 7.13 (t, 1H); 7.06 (b-s, 2H); 6.81 (b-s, 1H); 5.86 (s, 2H); 3.79 (d, 2H); 1.32 (s, 9H); 1.23-1.07 (m, 2H); 0.89-075 (m, 1H); 0.56-0.45 (m, 2H); 0.29-0.21 (m, 2H).

### Example 66

### 1-[(3,5-bis{[2-(methyloxy)ethyl]oxy}phenyl)methyl]-3-[4-(1,1-dimethylethyl)phenyl]-1H-indole-2-carboxylic acid

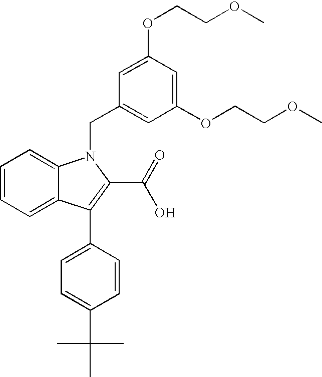

To a solution of 350 mg of ester Intermediate 31 in 3.5 mL of THF and 1.0 mL of water was added 0.5 gram of solid NaOH (pellet). The mixture was stirred with heating to ˜80° C. overnight (14 h). An additional 400 mg of NaOH was added and stirring was continued at 90° C. for 90 minutes. Cooled, added 2 mL H₂O, then added concentrated HCl to pH˜5. Added 20 mL EtOAc and 5 mL H₂O and extracted the aqueous phase with EtOAc. The organics were washed with brine, dried over MgSO₄, filtered and concentrated. The residue was taken into hot MeOH (2 mL) before allowing the solution to stand in the freezer for 2 h. The resulting white solids were isolated by filtration and dried in a vacuum oven at ˜60° C. for several hrs to yield 290 mg (90% yield) of the title compound as a white solid. [¹H NMR (300 MHz, CDCl₃) δ 7.60 (d, 1H, J=8.1 Hz), 7.48 (m, 4H), 7.34 (d, 2H, J=3.8 Hz), 7.14 (m, 1H), 6.38 (d, 1H, J=2.0 Hz), 6.32 (d, 2H, J=2.1 Hz), 5.78 (s, 2H), 4.01 (m, 4H), 3.67 (m, 4H), 3.41 (s, 6H), 1.41 (s, 9H)].

Alternative syntheses of Example 66:

Route 2:

Ethyl 3-(4-tert-butylphenyl)-1H-indole-2-carboxylate (40 g, 0.125 mol) and KOtBu (17.6 g, 0.157 mol) were combined in DMA (320 mL). 1-(bromomethyl)-3,5-difluorobenzene (19.1 mL, 0.149 mol) was added and the reaction mixture was stirred at room temperature for 3 h. A solution of KOH (8.4 g, 0.15 mol) in water (120 mL) was added and the reaction mixture was heated at 60° C. overnight. Additional KOH (4.2 g, 0.075 mol) in water (40 mL) was added and heating at 60° C. was continued for an additional 4.5 h. After cooling to room temperature, water (120 mL) followed by conc. HCl (80 mL) were added slowly, keeping the reaction temperature below 30° C. during the additions. After stirring at room temperature overnight, the solids were filtered off, washed with water, and dried under vacuum (26 in Hg, 54° C.) to provide 49.7 g of 1-[(3,5-difluorophenyl)methyl]-3-[4-(1,1-dimethylethyl)phenyl]-1H-indole-2-carboxylic acid (94%) as a white solid.

To a slurry of KOtBu (24.1 g, 0.215 mol) in toluene (20 mL), 2-methoxyethanol (19.1 mL, 0.238 mol) was slowly added and the reaction mixture was heated to 80° C. for ˜30 minutes. Meanwhile, 1-[(3,5-difluorophenyl)methyl]-3-[4-(1,1-dimethylethyl)phenyl]-1H-indole-2-carboxylic acid (5.0 g, 0.012 mol), toluene (7.5 mL), and DMPU (10 mL) were stirred at room temperature until homogeneous. This solution was then added to the alkoxide solution and the reaction mixture was heated at 80° C. overnight. After cooling to room temperature, the reaction mixture was washed with water (25 mL) and 10% brine (3×25 mL). The organic layer was heated to 60° C., 6N HCl (15 mL) was added, and the layers were separated. The organic layer was cooled to 20° C. and heptane (50 mL) was added. After further cooling to 0° C. for 2-3 hours, the solids were filtered off, washed with heptane, and dried under vacuum (25 in Hg, 50° C.) to provide 5.3 g (84%) of the title compound (Example 66) as a white solid.

Route 3:

Ethyl 3-(4-tert-butylphenyl)-1H-indole-2-carboxylate (3.1 g, 9.66 mmol), and Cs₂CO₃ (8.61 g, 24.4 mmol) were combined in DMF (10 mL). 1-(bromomethyl)-3,5-difluorobenzene (2.04 g, 9.9 mmol) was added and the reaction mixture was heated to 80° C. for 90 minutes. Water and MTBE were added. The organic layer was washed with additional water, dried over MgSO₄ and concentrated under vacuum to provide 4.38 g of ethyl 1-[(3,5-difluorophenyl)methyl]-3-[4-(1,1-dimethylethyl)phenyl]-1H-indole-2-carboxylate (99%) as a thick oil.

KOtBu (1.23 g, 11.0 mmol), DME (1 mL), and 2-methoxyethanol (1.3 mL, 16.4 mmol) were combined. Ethyl 1-[(3,5-difluorophenyl)methyl]-3-[4-(1,1-dimethylethyl)phenyl]-1H-indole-2-carboxylate (0.5 g, 1.12 mmol) was added and the reaction mixture was heated to 80° C. for 16 h. The reaction mixture was cooled and 6N HCl was added until the pH=1. Water was then added until a precipitate formed and the slurry was cooled in an ice bath. The solids were filtered off, washed with water, and dried in a vacuum oven (50° C., 26 in Hg).

The product was recrystallized from acetone (2 mL)/heptane (6 mL) and cooled in a refrigerator overnight. The solids were filtered off, washed with heptane, and dried in a vacuum oven (50° C., 26 in Hg) overnight to produce 0.43 g (78%) of ester Intermediate 31. The conversion of ester Intermediate 31 to the title acid compound Example 66 has been described above.

### Example 67

### 3-[4-(1,1-dimethylethyl)phenyl]-1-({3-{[2-(methyloxy)ethyl]oxy}-5-[(phenylmethyl)oxy]phenyl}methyl)-1H-indole-2-carboxylic acid

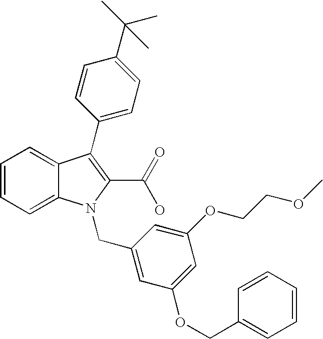

To a solution of Ethyl 3-[4-(1,1-dimethylethyl)phenyl]-1-({3-{[2-(methyloxy)ethyl]oxy}-5-[(phenylmethyl)oxy]phenyl}methyl)-1H-indole-2-carboxylate (Intermediate 32, 170 mg) in 1 mL EtOH, 2 mL THF, and 1 mL water was added 140 mg NaOH pellet. Stirred 14 hr at 80° C., cooled, and acidified to pH˜2 with conc. HCL solution. Diluted into 3 mL EtOAc, aqueous phase was extracted, and the combined organics dried over Na₂SO₄, filtered, and concentrated to yield 86 mg of the title compound as a tan colored solid after drying under vacuum. LC/MS 474.36 (MH+, 50%); 1H NMR (400 MHz, CDCL₃) δ 7.59 (d, 1H, J=8.2 Hz), 7.46 (d, 2H, J=6.4 Hz), 7.44 (d, 2H, J=6.2 Hz), 7.31 (m, 7H), 7.12 (m, 1H), 6.41 (d, 1H, J=1.8 Hz), 6.33 (s, 1H), 6.29 (s, 1H), 5.74 (s, 2H), 4.93 (s, 2H), 3.97 (m, 2H), 3.64 (m, 2H), 3.38 (s, 3H), 1.38 (s, 9H).

### Example 68

### 3-[4-(1,1-dimethylethyl)phenyl]-1-{[3-{[2-(methyloxy)ethyl]oxy}-5-(4-morpholinyl)phenyl]methyl}-1H-indole-2-carboxylic acid

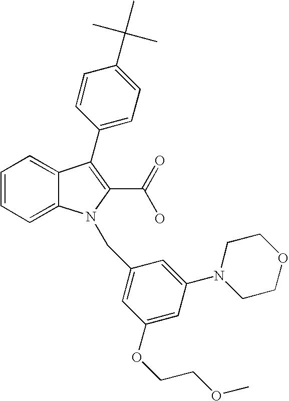

To 165 mg (0.26 mmol) of Intermediate 36 (Ethyl 3-[4-(1,1-dimethylethyl)phenyl]-1-[(3-{[2-(methyloxy)ethyl]oxy}-5-{[(trifluoromethyl)sulfonyl]oxy}phenyl)methyl]-1H-indole-2-carboxylate) in 2 mL of toluene was added 27 uL (0.31 mmol) of morpholine, 2 mg (0.008 mmol) of Pd(OAc)₂, 7 mg (0.012 mmol) of BINAP, and 120 mg (0.36 mmol) of Cs₂CO₃. The mixture was stirred under N₂ at 80° C. for 16 hr. Another 2 mg (0.008 mmol) of Pd(OAc)₂, 7 mg (0.012 mmol) of BINAP, and 120 mg (0.36 mmol) of Cs₂CO₃ were added and the mixture stirred at 80° C. for an additional 24 hr. The solution was filtered through a pad of Celite and the pad washed with 25 mL of EtOAc. The combined filtrates were washed with 25 mL of H₂O and 25 mL of brine. The organics were then concentrated and the residue purified by silica gel chromatography (40 grams of silica gel eluting with 0-60% EtOAc in hexanes over 45 minutes.) The fractions containing product were concentrated and the residue was taken up in a mixture of 2 mL THF, 1 mL of EtOH, and 1 mL H₂O. To this solution was added 64 mg (1.59 mmol) of NaOH and the solution stirred at 50° C. for 16 hr. Another 0.5 mL of H₂O and 80 mg of NaOH were added and the solution stirred at 60° C. for an additional 2 hr. The solution was added dropwise to 5 mL of 0.5 N HCl (aq) then extracted with two 10 mL portions of EtOAc. The combined organics were washed with 10 mL of H₂O and 10 mL of brine then dried over 0.5 g of Na₂SO₄. The solution was concentrated to give 58 mg (41%) of the title compound as a white solid: 1H NMR (400 MHz, DMSO-d6) δ 12.99 (s, 1H), 7.60 (d, 1H, J=8.4 Hz), 7.47-7.44 (m, 3H), 7.36 (d, 2H, J=6.1 Hz), 7.31-7.27 (m , 1H), 7.11-7.07 (m, 1H), 6.39 (s, 1H), 6.31 (s, 1H), 5.98 (s, 1H), 5.69 (s, 2H), 3.92-3.90 (m, 2H), 3.67-3.65 (m, 4H), 3.53-3.50 (m, 2H), 3.21 (s, 3H), 3.03-2.99 (m, 4H), 1.32 (s, 9H); MS (ESI) m/z 543 (MH+). Example 68 may also prepared from a crude THF solution of Intermediate 35:

A THF (900 mL) solution of the crude material prepared as Intermediate 35 was diluted with MeOH (900 mL) and 500 mL of 5N NaOH solution was added over 2 min. Heated with stirring at 64° C. (reflux) for 2 hrs. Added 6N HCl over 5 min, cooled, added 1 L EtOAc, then back extracted the aqueous phase (1×500 mL) with EtOAc. The combined organics were washed with water (2×500 mL), dried over Na₂SO₄, filtered, then stirred over 15 g DARCO-G60 decolorizing charcoal for 30 min and filtered through celite. The solution was concentrated, taken into 375 mL acetonitrile with heating, then stirred over several days at ambient temperature. The resulting solids were isolated via filtration washing with acetonitrile and dried in a vacuum oven at 60° C. overnight to yield 64 g of the title compound (Example 68: 3-[4-(1,1-dimethylethyl)phenyl]-1-{[3-{[2-(methyloxy)ethyl]oxy}-5-(4-morpholinyl)phenyl]methyl}-1H-indole-2-carboxylic acid) as a partial hydrochloride salt as determined by elemental anaysis: Anal Calcd for C₃₃H₃₈N₂O₅ (0.75 HCl), Found C, 69.28; H, 6.82; N, 4.87; Cl 4.5: Calcd C, 69.53; H, 6.85; N, 4.91; Cl, 4.61. 1H NMR (400 MHz, DMSO-d6) δ 7.60 (d, 1H, J=8.3 Hz), 7.47-7.44 (m, 3H), 7.36 (d, 3H, J=6.1 Hz), 7.29 (m, 1H), 7.09 (t, 1H, J=7.4 Hz), 6.41 (s, 1H), 6.34 (s, 1H), 5.99 (s, 1H), 5.69 (s, 2H), 4.8 (brs, 1H), 3.91 (t, 2H, J=4.5 Hz), 3.67 (t, 4H, J=4.3 Hz), 3.52 (t, 2H, J=4.2 Hz), 3.21 (s, 3H), 3.02 (t, 4H, J=4.5 Hz), 1.32 (s, 9H).

Biological Section

In Vitro Evaluation:

Plasmids—PCR primers containing Kpnl and BamHI restriction sites were used to amplify PPARγ ligand binding domain (LBD) fragment (172-475) from a full-length human clone. The LBD fragment was ligated into the multiple cloning site of pFA-CMV (Stratagene). The resulting construct (pFA-CMV-GAL4-hPPARγLBD carried a fusion of the LBD with the yeast-derived GAL4 DNA-binding domain under the control of the CMV immediate early promoter. Reporter construct UAStkLuc carries a single 17 bp (CGGAGTACTGTCCTCCG) upstream activating sequence (UAS), the tk minimal promoter, and the firefly luciferase gene. The integrity of each construct was confirmed by diagnostic restriction digestion and by sequencing. Plasmid DNA was prepared using Qiagen Maxi-Prep kits.

PPARγ Cell-based luciferase assay—African Green Monkey kidney cell line CV-1 (ATCC CCL-70) was maintained in Dulbecco's Modified Eagle's Medium (D-MEM) containing 10% fetal bovine serum, 2 mM glutamine, and 1% penicillin/streptomycin (pen/strep). In preparation for luciferase assays, CV-1 cells were grown in charcoal-stripped cell medium containing D-MEM/F-12 medium supplemented with 5% or 3% dextran-treated/charcoal-stripped (CS) fetal bovine serum, 2 mM glutamine, with or without 1% pen/strep, as described below. CS fetal bovine serum was purchased from Hyclone; all other cell culture reagents were from Gibco.

The luciferase protocol is a multi-day procedure. On day 1, confluent cells in maintenance medium were subcultured 1:10 into T-175 cm² flasks containing 50 mL of 3% CS medium with pen/strep. These flasks were allowed to incubate at 5% CO₂ and 37° C. for 72 hours.

Cells were harvested by trypsinization and then transfected using FuGENE (Roche) according to the manufacturer's specifications. Briefly, each transfection contained 0.55 μg pFA_CMV_GAL4_hPPARγ_LBD plasmid, 10.9 μg UAStkLUC, and 24 μg pBluescript (carrier DNA). Plasmid DNA was mixed with FuGENE in OptiMEM-1 medium and incubated for 30 min at room temperature. During this incubation, cells were harvested into 3% CS medium without pen-strep and dispensed at 14 million cells per T-175 cm² flask. Transfection mixes were added to the flasks and incubated overnight at 5% CO₂ and 37° C.

Transfected cells were added to 384-well plates containing pharmacological agents. Rosiglitazone standard was reconstituted in DMSO at 1 mM. For 11-point dose-response experiments, the compounds were 3-fold serially diluted in DMSO and then stamped to 384-well assay plates (NUNC, catalog #164564) at 0.5 μL/well using a Beckman FX. DMSO and agonist control compound Rosiglitazone (1 mM) were each stamped at 0.5 μL/well to columns 23 and 24, respectively, of the 384-well plates. Transfected cells were harvested in 5% CS medium with pen/strep and dispensed at 10,000 cells/well (50 μL) onto the prepared 384-well compound plates using a Titertek Multidrop. Following overnight incubation at 5% CO₂ and 37° C., Steady-Glo reagent (Promega) was added to the assay plates using a Multidrop. Plates were incubated for 10 min to ensure complete cell lysis and read in a ViewLux (PerkinElmer). Examples 1-68 all showed partial agonism of the hPPARγ receptor in this in vitro PPARγ Cell-based luciferase assay described immediately above. Partial agonism is defined here as 20-80% activation (relative to full agonist rosiglitazone) at concentrations of 10⁻⁶ M or less.

In Vivo Evaluation:

Male Zucker Diabetic Fatty rats were lightly anesthetized with isofluorane gas and bleed by tail vein to obtain postprandial baseline concentrations for serum glucose, serum lipids and insulin. Animals were baseline matched by serum glucose and randomized into vehicle or treatment groups with compound administration by oral gavage beginning at 6.5 weeks of age. Selected compounds were administered once daily at 10 mg/kg in 25 mM N-Methyl-Glucamine with 5% w/v Solutol HS15. After 28 consecutive days of treatment, blood samples were obtained and analyzed for serum glucose. Values in Table 1 for % Glucose reduction represent a summary of the percent reduction from vehicle control animals at day 28 relative to normalization defined in this model as serum glucose levels of 140 mg/dL.

TABLE 1
Biological activity

| Example | % Glucose Reduction |
|---|---|
| 2 | 96 |
| 11 | 49 |
| 18 | 44 |
| 42 | 82 |
| 24 | 79 |
| 68 | 95 |

Although specific embodiments of the present invention are herein illustrated and described in detail, the invention is not limited thereto. The above detailed descriptions are provided as exemplary of the present invention and should not be construed as constituting any limitation of the invention. Modifications will be obvious to those skilled in the art, and all modifications that do not depart from the spirit of the invention are intended to be included within the scope of the appended claims.

What is claimed is:

1. A compound of formula (I) 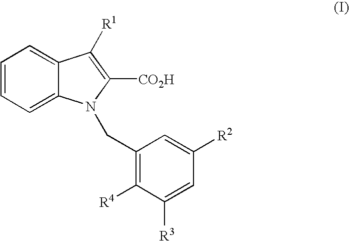
or salt or solvate thereof, wherein;
R¹ is —O-Ph-C₁₋₆alkyl, —NH-Ph-C₁₋₆alkyl, —CH₂-Ph-haloC₁₋₆alkyl, aryl or heterocyclyl, wherein said aryl or heterocyclyl is optionally mono-substituted with R⁷;
R² is C₁₋₆haloalkyl, R^{a}—R^{b}—R^{c}, heterocyclyl or aryl, wherein said aryl is optionally substituted with R⁸ and said heterocyclyl is optionally substituted with R⁹;
R³ is H, C₁₋₆haloalkyl, or R^{a}—R^{b}—R^{c};
R^{a} is —O—;
R^{b} is a bond, C₁₋₆alkylene or —C(O)—;
R^{c} is H, C₁₋₆alkyl, aryl, C₃₋₆cycloalkyl, C₁₋₆alkoxy, —NR⁵R⁶, —O(CH₂)₂OCH₃, or heterocyclyl optionally substituted with ═O or C₁₋₆alkyl; wherein
when R^{b} is a bond, R^{c} is H or C₁₋₆alkyl;
R⁴ and R⁵ are each independently H or C₁₋₆alkyl; wherein
when R³ and R⁴ are both H, R² is optionally substituted aryl or optionally substituted heterocyclyl;
R⁶ is C₁₋₆alkyl, or thienylC₁₋₆alkylene;
R⁷ is C₁₋₆alkyl, —C(O)CH₃, C₁₋₆alkoxy, or haloC₁₋₆alkyl;
R⁸ is —OH, —CO₂H, —OC₁₋₆alkylenephenyl, C₁₋₆alkoxy, —SC₁₋₆alkyl, —S(O)₂C₁₋₆alkyl, —C(O)NR⁵R⁶, or —OC(CH₃)₂CO₂H; and
R⁹ is —C(O)CH₃, —C(O)OC₁₋₆alkyl, —C(O)O(CH₂)₂OCH₃, —C(O)NH₂, —S(O)₂C₁₋₆alkyl, —S(O)₂NH₂, or —S(O)₂NC(O)OC₁₋₆alkyl.

2. A compound as claimed in claim 1, wherein the compound is a compound of formula (II) 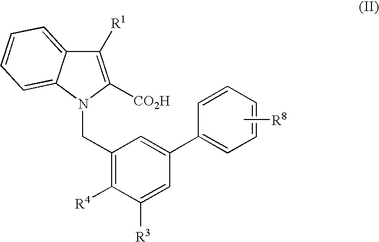
or a salt or solvate thereof, wherein
R¹ is —O-Ph-C₁₋₆alkyl, —NH-Ph-C₁₋₆alkyl, —CH₂-Ph-haloC₁₋₆alkyl, aryl or heterocyclyl, wherein said aryl or heterocyclyl is optionally mono-substituted with R⁷;
R³ is H, C₁₋₆haloalkyl, or R^{a}—R^{b}—R^{c};
R^{a} is —O—;
R^{b} is a bond, C₁₋₆alkylene or —C(O)—;
R^{c} is H, C₁₋₆alkyl, aryl, C₃₋₆cycloalkyl, C₁₋₆alkoxy, —NR⁵R⁶, —O(CH₂)₂OCH₃, or heterocyclyl optionally substituted with ═O or C₁₋₆alkyl;
wherein
when R^{b} is a bond, R^{c} is H or C₁₋₆alkyl;
R⁴ and R⁵ are each independently H or C₁₋₆alkyl;
R⁶ is C₁₋₆alkyl, or thienylC₁₋₆alkylene;
R⁷ is C₁₋₆alkyl, —C(O)CH₃, C₁₋₆alkoxy, or haloC₁₋₆alkyl; and
R⁸ is —OH, —CO₂H, —CO₂H, —OC₁₋₆alkylenephenyl, C₁₋₆alkoxy, —SC₁₋₆alkyl, —S(O)₂C₁₋₆alkyl, —C(O)NR⁵R⁶, or —OC(CH₃)₂CO₂H.

3. A compound as claimed in claim 1, wherein the compound is a compound of formula (III) 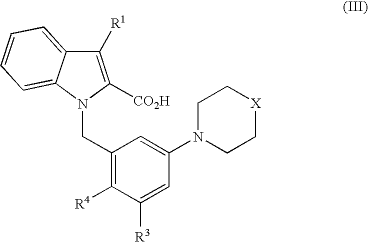
or a salt or solvate thereof, wherein
X is O, S, S(O)₂, or N—R⁹;
R¹ is —O-Ph-C₁₋₆alkyl, —NH-Ph-C₁₋₆alkyl, —CH₂-Ph-haloC₁₋₆alkyl, aryl or heterocyclyl, wherein said aryl or heterocyclyl is optionally mono-substituted with R⁷;
R³ is H, OH, C₁₋₆haloalkyl, C₁₋₆alkoxy, or R^{a}—R^{b}—R^{c};
R^{a} is —O—;
R^{b} is a bond, C₁₋₆alkylene or —C(O)—;
R^{c} is H, C₁₋₆alkyl, aryl, C₃₋₆cycloalkyl, C₁₋₆alkoxy, —NR⁵R⁶, —O(CH₂)₂OCH₃, or heterocyclyl optionally substituted with ═O or C₁₋₆alkyl;
wherein
when R^{b} is a bond, R^{c} is H or C₁₋₆alkyl;
R⁴ and R⁵ are each independently H or C₁₋₆alkyl;
R⁶ is C₁₋₆alkyl, or thienylC₁₋₆alkylene;
R⁷ is C₁₋₆alkyl, —C(O)CH₃, C₁₋₆alkoxy, or haloC₁₋₆alkyl; and
R⁹ is —C(O)CH₃, —C(O)OC₁₋₆alkyl, —C(O)O(CH₂)₂O CH₃, —S(O)₂C₁₋₆alkyl, —S(O)₂NH₂, or —S(O)₂NC(O)OC₁₋₆alkyl.

4. A compound as claimed in claim 1, wherein the compound is a compound of formula (IV) 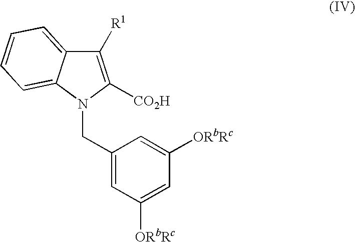
or a salt or solvate thereof, wherein
R¹ is —O-Ph-C₁₋₆alkyl, —NH-Ph-C₁₋₆alkyl, —CH₂-Ph-haloC₁₋₆alkyl, aryl or heterocyclyl, wherein said aryl or heterocyclyl is optionally mono-substituted with R⁷;
R^{b} is a bond, C₁₋₆alkylene or —C(O)—;
R^{c} is H, C₁₋₆alkyl, aryl, C₃₋₆cycloalkyl, C₁₋₆alkoxy, —NR⁵R⁶, —O(CH₂)₂O CH₃, or heterocyclyl optionally substituted with ═O or C₁₋₆alkyl; wherein
when R^{b} is a bond, R^{c} is H or C₁₋₆alkyl;
R⁵ is H or C₁₋₆alkyl;
R⁶ is C₁₋₆alkyl, or thienylC₁₋₆alkylene; and
R⁷ is C₁₋₆alkyl, —C(O)CH₃, C₁₋₆alkoxy, or haloC₁₋₆alkyl.

5. A compound as claimed in claim 1, wherein the compound is a compound of formula (V) 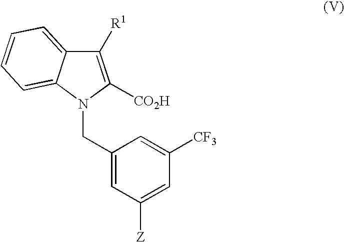
or a salt or solvate thereof, wherein
Z is CF₃ or OR^{b}R^{c};
R¹ is —O-Ph-C₁₋₆alkyl, —NH-Ph-C₁₋₆alkyl, —CH₂-Ph-haloC₁₋₆alkyl, aryl or heterocyclyl, wherein said aryl or heterocyclyl is optionally mono-substituted with R⁷;
R^{b} is C₁₋₆alkylene or —C(O)—;
R^{c} is H, C₁₋₆alkyl, aryl, C₃₋₆cycloalkyl, C₁₋₆alkoxy, —NR⁵R⁶, —O(CH₂)₂OCH₃, or heterocyclyl optionally substituted with ═O or C₁₋₆alkyl;
R⁵ is H or C₁₋₆alkyl;
R⁶ is C₁₋₆alkyl, or thienylC₁₋₆alkylene; and
R⁷ is C₁₋₆alkyl, —C(O)CH₃, C₁₋₆alkoxy, or haloC₁₋₆alkyl.

6. A compound as claimed in claim 4, wherein R¹ is —O-Ph-t-butyl, —NH-Ph-t-butyl, —CH₂-Ph-CF₃, phenyl, benzofuranyl, thiophenyl, or pyridinyl, wherein said phenyl, benzofuranyl, thiophenyl, or pyridinyl, is optionally mono-substituted with R⁷.

7. A compound as claimed in claim 1, wherein R^{e} is C₁₋₆alkyl, phenyl, cyclopropyl, CF₃, —NR⁵R⁶, —O(CH₂)₂OCH₃, oxoimidazolidinyl, piperazinyl, piperidinyl, morpholinyl, pyrrolyl, or pyrrolidinyl, wherein said piperazinyl, piperidinyl, morpholinyl, pyrrolyl, or pyrrolidinyl is optionally substituted with C₁₋₆alkyl.

8. A compound as claimed in claim 1, wherein R² is OH, C₁₋₆alkoxy, CF₃, R^{a}—R^{b}—R^{c}, phenyl, morpholinyl, piperazinyl, thiomorpholinyl, or dioxidothiomorpholinyl, wherein said phenyl is optionally substituted with R⁸ and said morpholinyl, piperazinyl, thiomorpholinyl, or dioxidothiomorpholinyl is optionally substituted with R⁹.

9. A compound as claimed in claim 4, wherein R¹ is optionally substituted phenyl.

10. The compound of claim 9 wherein said phenyl is optionally substituted with C₁₋₆alkyl.

11. The compound of claim 10 wherein said C₁₋₆alkyl is t-butyl.

12. A compound as claimed in claim 1 wherein at least one of R² and R³ is R^{a}—R^{b}—R^{c}.

13. The compound of claim 12 wherein R^{a} is —O—, R^{b} is C₁₋₃alkylene, and R^{c} is C₁₋₃alkoxy.

14. The compound of claim 13 wherein said C₁₋₃alkylene is ethylene and said C₁₋₃alkoxy is methoxy.

15. A compound selected from the group consisting of:
1-({3-[(cyclopropylmethyl)oxy]-5-[(phenylmethyl)oxy]phenyl}methyl)-3-[4-(1,1-dimethylethyl)phenyl]-1H-indole-2-carboxylic acid;
1-[(3-[(cyclopropylmethyl)oxy]-5-{[2-(methyloxy)ethyl]oxy}phenyl)methyl]-3-[4-(1,1-dimethylethyl)phenyl]-1H-indole-2-carboxylic acid;
1-({3-[(cyclopropylmethyl)oxy]-5-hydroxyphenyl}methyl)-3-[4-(1,1-dimethylethyl)phenyl]-1H-indole-2-carboxylic acid;
1-{[3-[(cyclopropylmethyl)oxy]-5-(methyloxy)phenyl]methyl}-3-[4-(1,1-dimethylethyl)phenyl]-1H-indole-2-carboxylic acid;
1-({3,5-bis[(cyclopropylmethyl)oxy]phenyl}methyl)-3-[4-(1,1-dimethylethyl)phenyl]-1H-indole-2-carboxylic acid;
1-({3-[(cyclopropylmethyl]-5-[(3-methylbutyl)oxy]phenyl}methyl)-3-[4-(1,1-dimethylethyl)phenyl]-1H-indole-2-carboxylic acid;
1-[(4′-carboxy-3-biphenylyl)methyl]-3-[4-(1,1-dimethylethyl)phenyl]-1H-indole-2-carboxylic acid;
3-[4-(1,1-dimethylethyl)phenyl]-1-({4′-[(phenylmethyl)oxy]-3-biphenylyl}methyl)-1H-indole-2-carboxylic acid;
3-[4-(1,1-dimethylethyl)phenyl]-1-[(4′-hydroxy-3-biphenylyl)methyl]-1H-indole-2-carboxylic acid;
3-[4-(1,1-dimethylethyl)phenyl]-1-[(4′-hydroxy-4-methyl-3-biphenylyl)methyl]-1H-indole-2-carboxylic acid;
1-[(4′-carboxy-4-methyl-3-biphenylyl)methyl]-3-[4-(1,1-dimethylethyl)phenyl]-1H-indole-2-carboxylic acid;
1-({4′-[(1-carboxy-1-methylethyl)oxy]-4-methyl-3-biphenylyl}methyl)-3-[4-(1,1-dimethylethyl)phenyl]-1H-indole-2-carboxylic acid;
3-[4-(1,1-dimethylethyl)phenyl]-1-{[4-methyl-4′-(methyloxy)-3-biphenylyl]methyl}-1H-indole-2-carboxylic acid;
3-(4-acetylphenyl)-1-[(4′-carboxy-4-methyl-3-biphenylyl)methyl]-1H-indole-2-carboxylic acid;
1-({4′-carboxy-5-[(cyclopropylmethyl)oxy]-3-biphenylyl}methyl)-3-[4-(1,1-dimethylethyl)phenyl]-1H-indole-2-carboxylic acid;
1-[(4′-hydroxy-3-biphenylyl)methyl]-3-[6-(methyloxy)-3-pyridinyl]-1H-indole-2-carboxylic acid;
3-[4-(1,1-dimethylethyl)phenyl]-1-{[4-methyl-3′-(methylthio)-3-biphenylyl]methyl}-1H-indole-2-carboxylic acid;
1-{[4′-carboxy-5-(methyloxy)-3-biphenylyl]methyl}-3-[4-(1,1-dimethylethyl)phenyl]-1H-indole-2-carboxylic acid;
1-({4′-carboxy-5-[(phenylmethyl)oxy]-3-biphenylyl}methyl)-3-[4-(1,1-dimethylethyl)phenyl]-1H-indole-2-carboxylic acid;
1-[(4′-carboxy-5-{[(methyloxy)methyl]oxy}-3-biphenylyl)methyl]-344-(1,1-dimethylethyl)phenyl]-1H-indole-2-carboxylic acid;
1-[(4′-carboxy-4-methyl]-3-biphenylyl)methyl]-3-[6-(methyloxy)-3-pyridinyl]-1H-indole-2-carboxylic acid;
1-[(4′-carboxy-5-hydroxy-3-biphenylyl)methyl]-3-[4-(1,1-dimethylethyl)phenyl]-1H-indole-2-carboxylic acid;
3-[4-(1,1-dimethylethyl)phenyl]-1-{[4′-(methylthio)-3-biphenylyl]methyl}-1H-indole-2-carboxylic acid;
3-[4-(1,1-dimethylethyl)phenyl]-1-{[4′-(methylsulfonyl)-3-biphenylyl]methyl}-1H-indole-2-carboxylic acid;
3-[4-(1,1-dimethylethyl)phenyl]-1-{[3′-(methylsulfonyl)-3-biphenylyl]methyl}-1H-indole-2-carboxylic acid;
3-[4-(1,1-dimethylethyl)phenyl]-1-{[3-(4-morpholinyl)phenyl]methyl}-1H-indole-2-carboxylic acid;
3-[4-(1,1-dimethylethyl)phenyl]-1-{[2-methyl-5-(4-morpholinyl)phenyl]methyl}-1H-indole-2-carboxylic acid;
1-({4′-[(dimethylamino)carbonyl]-4-methyl-3-biphenylyl}methyl)-3-[4-(1,1-dimethylethyl)phenyl]-1H-indole-2-carboxylic acid;
3-[4-(1,1-dimethylethyl)phenyl]-1-({4-methyl-3′-[(methylamino)carbonyl]-3-biphenylyl}methyl)-1H-indole-2-carboxylic acid;
3-[4-(1,1-dimethylethyl)phenyl]-1-[(4-methyl-3′-{[(2-thienylmethyl)amino]carbonyl}-3-biphenylyl)methyl]-1H-indole-2-carboxylic acid;
3-[4-(1,1-dimethylethyl)phenyl]-1-{[4-methyl-3′-({[2-(2-thienylmethyl]amino}carbonyl)-3-biphenylyl]methyl}-indole-2-carboxylic acid;
3-[4-(1,1-dimethylethyl)phenyl]-1-[(4-methyl-4′-{[(2-thienylmethyl)amino]carbonyl}-3-biphenylyl)methyl]-1H-indole-2-carboxylic acid;
3-[4-(1,1-dimethylethyl)phenyl]-1-{[4-methyl-4′-({[2-(2-thienyl)ethyl]amino}carbonyl)-3-biphenylyl]methyl}-1H-indole-2-carboxylic acid;
3-[4-(1,1-dimethylethyl)phenyl]-1-({3-[4-(methylsulfonyl)-1-piperazinyl]phenyl}methyl)-1H-indole-2-carboxylic acid;
1-{[3-(4-acetyl-1-piperazinyl)phenyl]methyl}-3-[4-(1,1-dimethylethyl)phenyl]-1H-indole-2-carboxylic acid;
3-[4-(1,1-dimethylethyl)phenyl]-1-[(3-{4-[(methyloxy)carbonyl]-1-piperazinyl}phenyl)methyl]-1H-indole-2-carboxylic acid;
1-({3-[4-(aminocarbonyl)-1-piperazinyl]phenyl}methyl)-3-[4-(1,1-dimethylethyl)phenyl]-1H-indole-2-carboxylic acid;
1-[(3-{4-[({[(1,1-dimethylethyl)oxy]carbonyl}amino)sulfonyl]-1-piperazinyl}phenyl)methyl]-3-[4-(1,1-dimethylethyl)phenyl]-1H-indole-2-carboxylic acid;
1-({3-[4-(aminosulfonyl)-1-piperazinyl]phenyl}methyl)-3-[4-(1,1-dimethylethyl)phenyl]-1H-indole-2-carboxylic acid;
1-[(3-[(cyclopropylmethyl)oxy]-5-{[2-(dimethylamino)ethyl]oxy}phenyl)methyl]-3-[4-(1,1-dimethylethyl)phenyl]-1H-indole-2-carboxylic acid;
1-[(3-[(cyclopropylmethyl)oxy]-5-{[2-(1-pyrrolidinyl)ethyl]oxy}phenyl)methyl]-3-[4-(1,1-dimethylethyl)phenyl]-1H-indole-2-carboxylic acid;
1-[(3-[(cyclopropylmethyl)oxy]-5-{[2-(4-morpholinyl)ethyl}oxy}phenyl)methyl]-3-[4-(1,1-dimethylethyl)phenyl]-1H-indole-2-carboxylic acid;
1-[(3-[(cyclopropylmethyl)oxy]-5-{[3-(dimethylamino)propyl]oxy}phenyl)methyl]-3-[4-(1,1-dimethylethyl)phenyl]-1H-indole-2-carboxylic acid;
3-[4-(1,1-dimethylethyl)phenyl]-1-{[3-(4-thiomorpholinyl)phenyl]methyl}-1H-indole-2-carboxylic acid;
3-[4-(1,1-dimethylethyl)phenyl]-1-{([3-(1,1-dioxido-4-thiomorpholinyl)phenyl]methyl}-1H-indole-2-carboxylic acid;
3-[4-(1,1-dimethylethyl)phenyl]-1-[(3-{4-[(ethyloxy)carbonyl]-1-piperazinyl}phenyl)methyl]-1H-indole-2-carboxylic acid;
3-[4-(1,1-dimethylethyl)phenyl]-1-{[3-(4-{[(1-methylethyl)oxy]carbonyl}-1-piperazinyl)phenyl]methyl}-1H-indole-2-carboxylic acid;
3-[4-(1,1-dimethylethyl)phenyl]-1-({3-[4-({[2-(methyloxy)ethyl]oxy}carbonyl)-1-piperazinyl]phenyl}methyl)-1H-indole-2-carboxylic acid;
1-[(3-{[(dimethylamino)carbonyl]oxy}-5-{[2-(methyloxy)ethyl]oxy}phenyl)methyl]-3-[4-(1,1-dimethylethyl)phenyl]-1H-indole-2-carboxylic acid;
3-[4-(1,1-dimethylethyl)phenyl]-1-[(3-{[2-(methyloxy)ethyl]oxy}-5-{[(4-methyl-1-piperazinyl)carbonyl]oxy}phenyl)methyl]-1H-indole-2-carboxylic acid;
3-[4-(1,1-dimethylethyl)phenyl]-1-({3-{[2-(methyloxy)ethyl]oxy}-5-[(1-piperidinylcarbonyl)oxy]phenyl}methyl)-1H-indole-2-carboxylic acid;
3-[4-(1,1-dimethylethyl)phenyl]-1-({3-{[2-(methyloxy)ethyl]oxy}-5-[(4-morpholinylcarbonyl)oxy]phenyl}methyl)-1H-indole-2-carboxylic acid;
344-(1,1-dimethylethyl)phenyl]-1-[(3-{[2-(methyloxy)ethyl]oxy}-5-{[(2-oxo-1-imidazolidinyl)carbonyl]oxy}phenyl)methyl]-1H-indole-2-carboxylic acid;
1-[(3-[(cyclopropylmethyl)oxy]-5-{[2-(1H-pyrrol-1-yl)ethyl]oxy}phenyl)methyl]-3-{[3-(trifluoromethyl)phenyl]methyl}-1H-indole-2-carboxylic acid;
1-({3-[(cyclopropylmethyl)oxy]-5-[(3-{[2-(methyloxy)ethyl]oxy}propyl)oxy]phenyl}methyl)-3-{[3-(trifluoromethyl)phenyl]methyl}-1H-indole-2-carboxylic acid;
1-[(3-[(cyclopropylmethyl)oxy]-5-{[2-(methyloxy)ethyl]oxy}phenyl)methyl]-3-{[3-(trifluoromethyl)phenyl]methyl}-1H-indole-2-carboxylic acid;
1-[(3-[(cyclopropylmethyl)oxy]-5-{[3-(dimethylamino)propyl]oxy}phenyl)methyl]-3-{[3-(trifluoromethyl)phenyl]methyl}-1H-indole-2-carboxylic acid hydrochloride;
1-[(3,5-bis{[2-(methyloxy)ethyl]oxy}phenyl)methyl]-3-{[3-(trifluoromethyl)phenyl]methyl}-1H-indole-2-carboxylic acid;
1-({3,5-bis[(cyclopropylmethyl)oxy]phenyl}methyl)-3-{[3-(trifluoromethyl)phenyl]methyl}-1H-indole-2-carboxylic acid;
3-(1-benzofuran-2-yl)-1-[(3,5-bis{[2-(methyloxy)ethyl]oxy}phenyl)methyl]-1H-indole-2-carboxylic acid;
1-[(3,5-bis{[2-(methyloxy)ethyl]oxy}phenyl)methyl]-3-{[4-(1,1-dimethylethyl)phenyl]oxy}-1H-indole-2-carboxylic acid;
1-[(3,5-bis{[2-(methyloxy)ethyl]oxy}phenyl)methyl]-3-{[4-(1,1-dimethylethyl)phenyl]amino}-1H-indole-2-carboxylic acid;
1-{[3,5-bis(trifluoromethyl)phenyl]methyl}-3-[4-(1,1-dimethylethyl)phenyl]-1H-indole-2-carboxylic acid;
3-[4-(1,1-dimethylethyl)phenyl]-1-{[3-{[2-(methyloxy)ethyl]oxy}-5-(trifluoromethyl)phenyl]methyl}-1H-indole-2-carboxylic acid;
1-{[3-[(cyclopropylmethyl)oxy]-5-(trifluoromethyl)phenyl]methyl}-3-[4-(1,1-dimethylethyl)phenyl]-1H-indole-2-carboxylic acid;
1-[(3,5-bis{[2-(methyloxy)ethyl]oxy}phenyl)methyl]-3-[4-(1,1-dimethylethyl)phenyl]-1H-indole-2-carboxylic acid;
3-[4-(1,1-dimethylethyl)phenyl]-1-({3-{[2-(methyloxy)ethyl]oxy}-5-[(phenylmethyl)oxy]phenyl}methyl)-1H-indole-2-carboxylic acid; and
3-[4-(1,1-dimethylethyl)phenyl]-1-{[3-{[2-(methyloxy)ethyl]oxy}-5-(4-morpholinyl)phenyl]methyl}-1H-indole-2-carboxylic acid; or a salt or solvate thereof.

16. A compound of claim 4, wherein said compound is a PPARγ modulator.

17. A pharmaceutical composition comprising a compound according to claim 4, and a pharmaceutically acceptable carrier.

18. A method for the treatment of hyperglycemia, type 2 diabetes, impaired glucose tolerance, insulin resistance, syndrome X, and dyslipidemia, comprising the administration of a compound according to claim 4.

19. (canceled)

20. (canceled)
